SEQUENCE LISTING (95 sequences)

SEQ ID NO: 1 - protein, 10 aa, Artificial
  CA170_1519 CDRH1
GFTFSNYGMV

SEQ ID NO: 2 - protein, 17 aa, Artificial
  CA170_1519 CDRH2
YIDSDGDNTYYRDSVKG

SEQ ID NO: 3 - protein, 8 aa, Artificial
  CA170_1519 CDRH3
GIVRPFLY

SEQ ID NO: 4 - protein, 16 aa, Artificial
  CA170_1519 CDRL1
KSSQSLVGASGKTYLY

SEQ ID NO: 5 - protein, 7 aa, Artificial
  CA170_1519 CDRL2
LVSTLDS

SEQ ID NO: 6 - protein, 9 aa, Artificial
  CA170_1519 CDRL3
LQGTHFPHT

SEQ ID NO: 7 - protein, 112 aa, Artificial
  Rat Ab 1519 VL region
DVVMTQTPLSLSVALGQPASISCKSSQSLVGASGKTYLYWLFQRSGQSPKRLIYLVSTLDSGIPDRFSGSGAETDFTLKIRRVEADDLGVYYCLQGTHFPHTFGAGTKLELK

SEQ ID NO: 8 - DNA, 336 bp, Artificial
  Rat Ab 1519 VL region
gatgttgtgatgacccagactccactgtctttgtcggttgcccttggacaaccagcctccatctcttgcaagtcaagtcagagcctcgtaggtgctagtggaaagacatatttgtattggttatttcagaggtccggccagtctccaaagcgactaatctatctggtgtccacactggactctggaattcctgataggttcagtggcagtggagcagagacagattttactcttaaaatccgcagagtggaagccgatgatttgggagtttattactgcttgcaaggtacacattttcctcacacgtttggagctgggaccaagctggaattgaaa

SEQ ID NO: 9 - protein, 132 aa, Artificial
  Rat Ab 1519 VL region with signal sequence
MMSPAQFLFLLMLWIQGTSGDVVMTQTPLSLSVALGQPASISCKSSQSLVGASGKTYLYWLFQRSGQSPKRLIYLVSTLDSGIPDRFSGSGAETDFTLKIRRVEADDLGVYYCLQGTHFPHTFGAGTKLELK

SEQ ID NO: 10 - DNA, 396 bp, Artificial
  Rat Ab 1519 VL region with signal sequence
atgatgagtcctgcccagttcctgtttctgctgatgctctggattcagggaaccagtggtgatgttgtgatgacccagactccactgtctttgtcggttgcccttggacaaccagcctccatctcttgcaagtcaagtcagagcctcgtaggtgctagtggaaagacatatttgtattggttatttcagaggtccggccagtctccaaagcgactaatctatctggtgtccacactggactctggaattcctgataggttcagtggcagtggagcagagacagattttactcttaaaatccgcagagtggaagccgatgatttgggagtttattactgcttgcaaggtacacattttcctcacacgtttggagctgggaccaagctggaattgaaa

SEQ ID NO: 11 - protein, 116 aa, Artificial
  Rat Ab 1519 VH region
EVPLVESGGGSVQPGRSMKLSCVVSGFTFSNYGMVWVRQAPKKGLEWVAYIDSDGDNTYYRDSVKGRFTISRNNAKSTLYLQMDSLRSEDTATYYCTTGIVRPFLYWGQGTTVTVS

SEQ ID NO: 12 - DNA, 348 bp, Artificial
  Rat Ab 1519 VH region
gaggtgccgctggtggagtctgggggcggctcagtgcagcctgggaggtccatgaaactctcctgtgtagtctcaggattcactttcagtaattatggcatggtctgggtccgccaggctccaaagaagggtctggagtgggtcgcatatattgattctgatggtgataatacttactaccgagattccgtgaagggccgattcactatctccagaaataatgcaaaaagcaccctatatttgcaaatggacagtctgaggtctgaggacacggccacttattactgtacaacagggattgtccggccctttctctattggggccaaggaaccacggtcaccgtctcg

SEQ ID NO: 13 - protein, 135 aa, Artificial
  Rat Ab 1519 VH region with signal sequence
MDISLSLAFLVLFIKGVRCEVPLVESGGGSVQPGRSMKLSCVVSGFTFSNYGMVWVRQAPKKGLEWVAYIDSDGDNTYYRDSVKGRFTISRNNAKSTLYLQMDSLRSEDTATYYCTTGIVRPFLYWGQGTTVTVS

SEQ ID NO: 14 - DNA, 405 bp, Artificial
  Rat Ab 1519 VH region with signal sequence
atggacatcagtctcagcttggctttccttgtccttttcataaaaggtgtccggtgtgaggtgccgctggtggagtctgggggcggctcagtgcagcctgggaggtccatgaaactctcctgtgtagtctcaggattcactttcagtaattatggcatggtctgggtccgccaggctccaaagaagggtctggagtgggtcgcatatattgattctgatggtgataatacttactaccgagattccgtgaagggccgattcactatctccagaaataatgcaaaaagcaccctatatttgcaaatggacagtctgaggtctgaggacacggccacttattactgtacaacagggattgtccggccctttctctattggggccaaggaaccacggtcaccgtctcg

SEQ ID NO: 15 - protein, 112 aa, Artificial [sequence omitted]

SEQ ID NO: 16 - DNA, 336 bp, Artificial
  1519 gL20 V-region (E. coli expression)
gatatccagatgacccagagtccaagcagtctctccgccagcgtaggcgatcgtgtgactattacctgtaaaagctcccagtccctggtgggtgcaagcggcaaaacctacctgtactggctcttccagaaaccgggcaaagctccgaaacgcctgatctatctggtgtctaccctggatagcggtattccgtctcgtttctccggtagcggtagcggtaccgaattcacgctgaccattagctccctccagccggaggactttgctacctattactgcctccagggcactcattttccgcacactttcggccagggtaccaaactggaaatcaaa

SEQ ID NO: 17 - DNA, 336 bp, Artificial
  1519 gL20 V-region (mammalian expression)
gatatccagatgacccagagcccatctagcttatccgcttccgttggtgatcgcgtgacaattacgtgtaagagctcccaatctctcgtgggtgcaagtggcaagacctatctgtactggctctttcagaagcctggcaaggcaccaaaacggctgatctatctggtgtctacccttgactctgggataccgtcacgattttccggatctgggagcggaactgagttcacactcacgatttcatcgctgcaacccgaggactttgctacctactactgcctgcaaggcactcatttccctcacactttcggccaggggacaaaactcgaaatcaaa

SEQ ID NO: 18 - protein, 133 aa, Artificial
  1519 gL20 V-region with signal sequence (E. coli expression)
MKKTAIAIAVALAGFATVAQADIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIK

SEQ ID NO: 19 - DNA, 399 bp, Artificial
  1519 gL20 V-region with signal sequence (E. coli expression)
atgaaaaagacagctatcgcaattgcagtggccttggctggtttcgctaccgtagcgcaagctgatatccagatgacccagagtccaagcagtctctccgccagcgtaggcgatcgtgtgactattacctgtaaaagctcccagtccctggtgggtgcaagcggcaaaacctacctgtactggctcttccagaaaccgggcaaagctccgaaacgcctgatctatctggtgtctaccctggatagcggtattccgtctcgtttctccggtagcggtagcggtaccgaattcacgctgaccattagctccctccagccggaggactttgctacctattactgcctccagggcactcattttccgcacactttcggccagggtaccaaactggaaatcaaa

SEQ ID NO: 20 - protein, 132 aa, Artificial
  1519 gL20 V-region with signal sequence (mammalian expression)
MSVPTQVLGLLLLWLTDARCDIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIK

SEQ ID NO: 21 - DNA, 396 bp, Artificial
  1519 gL20 V-region with signal sequence (mammalian expression)
atgtctgtccccacccaagtcctcggactcctgctactctggcttacagatgccagatgcgatatccagatgacccagagcccatctagcttatccgcttccgttggtgatcgcgtgacaattacgtgtaagagctcccaatctctcgtgggtgcaagtggcaagacctatctgtactggctctttcagaagcctggcaaggcaccaaaacggctgatctatctggtgtctacccttgactctgggataccgtcacgattttccggatctgggagcggaactgagttcacactcacgatttcatcgctgcaacccgaggactttgctacctactactgcctgcaaggcactcatttccctcacactttcggccaggggacaaaactcgaaatcaaa

SEQ ID NO: 22 - protein, 219 aa, Artificial
  1519 gL20 light chain (V + constant)
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 657 bp, Artificial
  1519 gL20 light chain (V + constant, E. coli expression)
gatatccagatgacccagagtccaagcagtctctccgccagcgtaggcgatcgtgtgactattacctgtaaaagctcccagtccctggtgggtgcaagcggcaaaacctacctgtactggctcttccagaaaccgggcaaagctccgaaacgcctgatctatctggtgtctaccctggatagcggtattccgtctcgtttctccggtagcggtagcggtaccgaattcacgctgaccattagctccctccagccggaggactttgctacctattactgcctccagggcactcattttccgcacactttcggccagggtaccaaactggaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcaccagtaacaaaaagttttaatagaggggagtgt

SEQ ID NO: 24 - DNA, 657 bp, Artificial
  1519 gL20 light chain (V + constant, mammalian expression)
gatatccagatgacccagagtccaagcagtctctccgccagcgtaggcgatcgtgtgactattacctgtaaaagctcccagtccctggtgggtgcaagcggcaaaacctacctgtactggctcttccagaaaccgggcaaagctccgaaacgcctgatctatctggtgtctaccctggatagcggtattccgtctcgtttctccggtagcggtagcggtaccgaattcacgctgaccattagctccctccagccggaggactttgctacctattactgcctccagggcactcattttccgcacactttcggccagggtaccaaactggaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 25 - protein, 240 aa, Artificial
  1519 gL20 light chain with signal sequence (E. coli expression)
MKKTAIAIAVALAGFATVAQADIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 720 bp, Artificial
  1519 gL20 light chain with signal sequence (E. coli expression)
atgaaaaagacagctatcgcaattgcagtggccttggctggtttcgctaccgtagcgcaagctgatatccagatgacccagagtccaagcagtctctccgccagcgtaggcgatcgtgtgactattacctgtaaaagctcccagtccctggtgggtgcaagcggcaaaacctacctgtactggctcttccagaaaccgggcaaagctccgaaacgcctgatctatctggtgtctaccctggatagcggtattccgtctcgtttctccggtagcggtagcggtaccgaattcacgctgaccattagctccctccagccggaggactttgctacctattactgcctccagggcactcattttccgcacactttcggccagggtaccaaactggaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcaccagtaacaaaaagttttaatagaggggagtgt

SEQ ID NO: 27 - protein, 239 aa, Artificial
  1519 gL20 light chain with signal sequence (mammalian expression)
MSVPTQVLGLLLLWLTDARCDIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 717 bp, Artificial
  1519 gL20 light chain with signal sequence (mammalian expression)
atgtctgtccccacccaagtcctcggactcctgctactctggcttacagatgccagatgcgatatccagatgacccagagcccatctagcttatccgcttccgttggtgatcgcgtgacaattacgtgtaagagctcccaatctctcgtgggtgcaagtggcaagacctatctgtactggctctttcagaagcctggcaaggcaccaaaacggctgatctatctggtgtctacccttgactctgggataccgtcacgattttccggatctgggagcggaactgagttcacactcacgatttcatcgctgcaacccgaggactttgctacctactactgcctgcaaggcactcatttccctcacactttcggccaggggacaaaactcgaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 29 - protein, 116 aa, Artificial
  1519 gH20 V-region
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVS

SEQ ID NO: 30 - DNA, 348 bp, Artificial
  1519 gH20 V-region (E. coli expression)
gaggttccgctggtcgagtctggaggcgggcttgtccagcctggagggagcctgcgtctctcttgtgcagtatctggcttcacgttctccaactacggtatggtgtgggttcgtcaggctccaggtaaaggtctggaatgggtggcgtatattgactccgacggcgacaacacctactatcgcgactctgtgaaaggtcgcttcaccatttcccgcgataacgccaaatccagcctgtacctgcagatgaacagcctgcgtgctgaagatactgcggtgtactattgcaccactggcatcgtgcgtccgtttctgtattggggtcagggtaccctcgttactgtctcg

SEQ ID NO: 31 - DNA, 348 bp, Artificial
  1519 gH20 V-region (mammalian expression)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcg

SEQ ID NO: 32 - protein, 137 aa, Artificial
  1519 gH20 V-region (E. coli expression)
MKKTAIAIAVALAGFATVAQAEVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVS

SEQ ID NO: 33 - DNA, 411 bp, Artificial
  1519 gH20 V-region (E. coli expression)
atgaagaagactgctatagcaattgcagtggcgctagctggtttcgccaccgtggcgcaagctgaggttccgctggtcgagtctggaggcgggcttgtccagcctggagggagcctgcgtctctcttgtgcagtatctggcttcacgttctccaactacggtatggtgtgggttcgtcaggctccaggtaaaggtctggaatgggtggcgtatattgactccgacggcgacaacacctactatcgcgactctgtgaaaggtcgcttcaccatttcccgcgataacgccaaatccagcctgtacctgcagatgaacagcctgcgtgctgaagatactgcggtgtactattgcaccactggcatcgtgcgtccgtttctgtattggggtcagggtaccctcgttactgtctcg

SEQ ID NO: 34 - protein, 135 aa, Artificial
  1519 gH20 V-region (mammalian expression)
MEWSWVFLFFLSVTTGVHSEVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVS

SEQ ID NO: 35 - DNA, 405 bp, Artificial
  1519 gH20 V-region with signal sequence (mammalian expression)
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcg

SEQ ID NO: 36 - protein, 228 aa, Artificial
  1519gH20 Fab′ heavy chain (V + human gamma-1 CH1 + hinge)
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 37 - DNA, 684 bp, Artificial
  1519gH20 Fab′ heavy chain (V + human gamma-1 CH1 + hinge, E.coli expression)
gaggttccgctggtcgagtctggaggcgggcttgtccagcctggagggagcctgcgtctctcttgtgcagtatctggcttcacgttctccaactacggtatggtgtgggttcgtcaggctccaggtaaaggtctggaatgggtggcgtatattgactccgacggcgacaacacctactatcgcgactctgtgaaaggtcgcttcaccatttcccgcgataacgccaaatccagcctgtacctgcagatgaacagcctgcgtgctgaagatactgcggtgtactattgcaccactggcatcgtgcgtccgtttctgtattggggtcagggtaccctcgttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcgccgcg

SEQ ID NO: 38 - DNA, 684 bp, Artificial
  1519gH20 Fab′ heavy chain (V + human gamma-1 CH1 + hinge, mammalian expression)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcgccgcg

SEQ ID NO: 39 - protein, 249 aa, Artificial
  1519 gH20 Fab′ heavy chain with signal sequence (E. coli expression)
MKKTAIAIAVALAGFATVAQAEVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 40 - DNA, 747 bp, Artificial
  1519 gH20 Fab′ heavy chain with signal sequence (E. coli expression)
atgaagaagactgctatagcaattgcagtggcgctagctggtttcgccaccgtggcgcaagctgaggttccgctggtcgagtctggaggcgggcttgtccagcctggagggagcctgcgtctctcttgtgcagtatctggcttcacgttctccaactacggtatggtgtgggttcgtcaggctccaggtaaaggtctggaatgggtggcgtatattgactccgacggcgacaacacctactatcgcgactctgtgaaaggtcgcttcaccatttcccgcgataacgccaaatccagcctgtacctgcagatgaacagcctgcgtgctgaagatactgcggtgtactattgcaccactggcatcgtgcgtccgtttctgtattggggtcagggtaccctcgttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcgccgcg

SEQ ID NO: 41 - protein, 247 aa, Artificial
  1519 gH20 Fab′ heavy chain with signal sequence (mammalian expression)
MEWSWVFLFFLSVTTGVHSEVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 42 - DNA, 741 bp, Artificial
  1519 gH20 Fab′ heavy chain with signal sequence (mammalian expression)
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcgccgcg

SEQ ID NO: 43 - protein, 444 aa, Artificial
  1519gH20 IgG4 heavy chain (V + human gamma-4P constant)
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - DNA, 1939 bp, Artificial
  1519gH20 IgG4 heavy chain (V + human gamma-4P constant with exons)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgcccagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccaccatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 45 - DNA, 1996 bp, Artificial
  1519gH20 IgG4 heavy chain (V + human gamma-4P constant) with signal sequence
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgcccagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccaccatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 46 - protein, 347 aa, Artificial
  1519gL20 FabFv light chain
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 47 - DNA, 1041 bp, Artificial
  1519gL20 FabFv light chain
gatatccagatgacccagagcccatctagcttatccgcttccgttggtgatcgcgtgacaattacgtgtaagagctcccaatctctcgtgggtgcaagtggcaagacctatctgtactggctctttcagaagcctggcaaggcaccaaaacggctgatctatctggtgtctacccttgactctgggataccgtcacgattttccggatctgggagcggaactgagttcacactcacgatttcatcgctgcaacccgaggactttgctacctactactgcctgcaaggcactcatttccctcacactttcggccaggggacaaaactcgaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctgagcagcaccctgacgctgtctaaagcagactacgagaaacacaaagtgtacgcctgcgaagtcacccatcagggcctgagctcaccagtaacaaaaagttttaatagaggggagtgtagcggtggcggtggcagtggtgggggaggctccggaggtggcggttcagacatacaaatgacccagagtccttcatcggtatccgcgtccgttggcgatagggtgactattacatgtcaaagctctcctagcgtctggagcaattttctatcctggtatcaacagaaaccggggaaggctccaaaacttctgatttatgaagcctcgaaactcaccagtggagttccgtcaagattcagtggctctggatcagggacagacttcacgttgacaatcagttcgctgcaaccagaggactttgcgacctactattgtggtggaggttacagtagcataagtgatacgacatttgggtgcggtactaaggtggaaatcaaacgtacc

SEQ ID NO: 48 - protein, 367 aa, Artificial
  1519gL20 FabFv light chain with signal sequence
MSVPTQVLGLLLLWLTDARCDIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 49 - DNA, 1101 bp, Artificial
  1519gL20 FabFv light chain with signal sequence
atgtctgtccccacccaagtcctcggactcctgctactctggcttacagatgccagatgcgatatccagatgacccagagcccatctagcttatccgcttccgttggtgatcgcgtgacaattacgtgtaagagctcccaatctctcgtgggtgcaagtggcaagacctatctgtactggctctttcagaagcctggcaaggcaccaaaacggctgatctatctggtgtctacccttgactctgggataccgtcacgattttccggatctgggagcggaactgagttcacactcacgatttcatcgctgcaacccgaggactttgctacctactactgcctgcaaggcactcatttccctcacactttcggccaggggacaaaactcgaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctgagcagcaccctgacgctgtctaaagcagactacgagaaacacaaagtgtacgcctgcgaagtcacccatcagggcctgagctcaccagtaacaaaaagttttaatagaggggagtgtagcggtggcggtggcagtggtgggggaggctccggaggtggcggttcagacatacaaatgacccagagtccttcatcggtatccgcgtccgttggcgatagggtgactattacatgtcaaagctctcctagcgtctggagcaattttctatcctggtatcaacagaaaccggggaaggctccaaaacttctgatttatgaagcctcgaaactcaccagtggagttccgtcaagattcagtggctctggatcagggacagacttcacgttgacaatcagttcgctgcaaccagaggactttgcgacctactattgtggtggaggttacagtagcataagtgatacgacatttgggtgcggtactaaggtggaaatcaaacgtacc

SEQ ID NO: 50 - protein, 357 aa, Artificial
  1519gH20 FabFv heavy chain
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGGGGTGGGGSEVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 51 - DNA, 1071 bp, Artificial
  1519gH20 FabFv heavy chain
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcgtccacaaagggcccatcggtcttccccctggcaccctcctccaagagcacctctgggggcacagcggccctgggctgcctggtcaaggactacttccccgaaccagtgacggtgtcgtggaactcaggtgccctgaccagcggcgttcacaccttcccggctgtcctacagtcttcaggactctactccctgagcagcgtggtgaccgtgccctccagcagcttgggcacccagacctacatctgcaacgtgaatcacaagcccagcaacaccaaggtcgataagaaagttgagcccaaatcttgtagtggaggtgggggctcaggtggaggcgggaccggtggaggtggcagcgaggttcaactgcttgagtctggaggaggcctagtccagcctggagggagcctgcgtctctcttgtgcagtaagcggcatcgacctgagcaattacgccatcaactgggtgagacaagctccggggaagtgtttagaatggatcggtataatatgggccagtgggacgaccttttatgctacatgggcgaaaggaaggtttacaattagccgggacaatagcaaaaacaccgtgtatctccaaatgaactccttgcgagcagaggacacggcggtgtactattgtgctcgcactgtcccaggttatagcactgcaccctacttcgatctgtggggacaagggaccctggtgactgtttcaagt

SEQ ID NO: 52 - protein, 376 aa, Artificial
  1519gH20 FabFv heavy chain with signal sequence
MEWSWVFLFFLSVTTGVHSEVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGGGGTGGGGSEVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 53 - DNA, 1128 bp, Artificial
  1519gH20 FabFv heavy chain with signal sequence
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcgtccacaaagggcccatcggtcttccccctggcaccctcctccaagagcacctctgggggcacagcggccctgggctgcctggtcaaggactacttccccgaaccagtgacggtgtcgtggaactcaggtgccctgaccagcggcgttcacaccttcccggctgtcctacagtcttcaggactctactccctgagcagcgtggtgaccgtgccctccagcagcttgggcacccagacctacatctgcaacgtgaatcacaagcccagcaacaccaaggtcgataagaaagttgagcccaaatcttgtagtggaggtgggggctcaggtggaggcgggaccggtggaggtggcagcgaggttcaactgcttgagtctggaggaggcctagtccagcctggagggagcctgcgtctctcttgtgcagtaagcggcatcgacctgagcaattacgccatcaactgggtgagacaagctccggggaagtgtttagaatggatcggtataatatgggccagtgggacgaccttttatgctacatgggcgaaaggaaggtttacaattagccgggacaatagcaaaaacaccgtgtatctccaaatgaactccttgcgagcagaggacacggcggtgtactattgtgctcgcactgtcccaggttatagcactgcaccctacttcgatctgtggggacaagggaccctggtgactgtttcaagt

SEQ ID NO: 54 - protein, 107 aa, Artificial
  Human VK1 2-1-(1) A30 JK2 acceptor framework
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 55 - DNA, 321 bp, Artificial
  Human VK1 2-1-(1) A30 JK2 acceptor framework
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccttacacttttggccaggggaccaagctggagatcaaa

SEQ ID NO: 56 - protein, 112 aa, Artificial
  Human VH3 1-3 3-07 JH4 acceptor framework
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARYFDYWGQGTLVTVS

SEQ ID NO: 57 - DNA, 336 bp, Artificial
  Human VH3 1-3 3-07 JH4 acceptor framework
gaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagatactttgactactggggccagggaaccctggtcaccgtctcc

SEQ ID NO: 58 - protein, 112 aa, Artificial
  Rat Ab 1548 VL region
DVVMTQTPLSLSVALGQPASISCKSSQSLVGASGKTYLYWLFQRSGQSPKRLIYLVSTLDSGIPDRFSGSGAETDFTLKIRRVEADDLGVYYCLQGTHFPHTFGAGTKLEIK

SEQ ID NO: 59 - DNA, 336 bp, Artificial
  Rat Ab 1548 VL region
gatgttgtgatgacccagactccactgtctttgtcggttgcccttggacaaccagcctccatctcttgcaagtcaagtcagagcctcgtaggtgctagtggaaagacatatttgtattggttatttcagaggtccggccagtctccaaagcgactaatctatctggtgtccacactggactctggaattcctgataggttcagtggcagtggagcagagacagattttactcttaaaatccgcagagtggaagccgatgatttgggagtttattactgcttgcaaggtacacattttcctcacacgtttggagctgggaccaagctggaaataaaa

SEQ ID NO: 60 - protein, 116 aa, Artificial
  Rat Ab 1548 VH region
EVPLVESGGGSVQPGRSMKLSCVVSGFTFSNYGMVWVRQAPKKGLEWVAYIDSDGDNTYYRDSVKGRFTISRNNAKSTLYLQMDSLRSEDTATYYCTTGIVRPFLYWGQGVMVTVS

SEQ ID NO: 61 - DNA, 348 bp, Artificial
  Rat Ab 1548 VH region
gaggtgccgctggtggagtctgggggcggctcagtgcagcctgggaggtccatgaaactctcctgtgtagtctcaggattcactttcagtaattatggcatggtctgggtccgccaggctccaaagaagggtctggagtgggtcgcatatattgattctgatggtgataatacttactaccgagattccgtgaagggccgattcactatctccagaaataatgcaaaaagcaccctatatttgcaaatggacagtctgaggtctgaggacacggccacttattactgtacaacagggattgtccggccctttctctattggggccaaggagtcatggtcacagtctcg

SEQ ID NO: 62 - protein, 112 aa, Artificial
  Rat Ab 1644 VL region
DVVMTQTPLSLSVAIGQPASISCKSSQSLVGASGKTYLYWLFQRSGQSPKRLIYLVSTLDSGIPDRFSGSGAETDFTLKIRRVEADDLGVYYCLQGTHFPHTFGAGTKLELK

SEQ ID NO: 63 - DNA, 336 bp, Artificial
  Rat Ab 1644 VL region
gatgttgtgatgacccagactccactgtctttgtcggttgccattggacaaccagcctccatctcttgcaagtcaagtcagagcctcgtaggtgctagtggaaagacatatttgtattggttatttcagaggtccggccagtctccaaagcgactaatctatctggtgtccacactggactctggaattcctgataggttcagtggcagtggagcagagacagattttactcttaaaatccgcagagtggaagccgatgatttgggagtttattactgcttgcaaggtacacattttcctcacacgtttggagctgggaccaagctggaactgaaa

SEQ ID NO: 64 - protein, 116 aa, Artificial
  Rat Ab 1644 VH region
EVPLVESGGGSVQPGRSTKLSCVVSGFTFSNYGMVWVRQAPKKGLEWVAYIGSDGDNIYYRDSVKGRFTISRNNAKSTLYLQMDSLRSEDTATYYCTTGIVRPFLYWGQGTTVTVS

SEQ ID NO: 65 - DNA, 348 bp, Artificial
  Rat Ab 1644 VH region
gaggtgccgctggtggagtctgggggcggctcagtgcagcctgggaggtccacgaaactctcctgtgtagtctcaggattcactttcagtaactatggcatggtctgggtccgccaggctccaaagaagggtctggagtgggtcgcatatattggttctgatggtgataatatttactaccgagattccgtgaagggtcgattcactatctccagaaataatgcaaaaagcaccctatatttgcaaatggacagtctgaggtctgaggacacggccacttattactgtacaacagggattgtccggccctttctctactggggccaaggaaccacggtcaccgtctcg

SEQ ID NO: 66 - protein, 112 aa, Artificial; identical to SEQ ID NO: 7
  Rat Ab 1496 VK region

SEQ ID NO: 67 - DNA, 336 bp, Artificial
  Rat Ab 1496 VK region
gatgttgtgatgacccagactccactgtctttgtcggttgcccttggacaaccagcctccatctcttgcaagtcaagtcagagcctcgtaggtgctagtggaaagacatatttgtattggttatttcagaggtccggccagtctccaaagcgactaatctatctggtgtccacactggactctggaattcctgataggttcagtggcagtggagcagagacagattttactcttaaaatccgcagagtggaagccgatgatttgggagtttattactgcttgcaaggtacacattttcctcacacgtttggagctgggaccaagctggaactgaaa

SEQ ID NO: 68 - protein, 116 aa, Artificial
  Rat Ab 1496 VH region
EVLLVESGGGSVQPGRSMKLSCVVSGFTFSNYGMVWVRQAPKKGLEWVAYIDSDGDNTYYRDSVKGRFTISRNNAKSTLYLQMDSLRSEDTATYYCTTGIVRPFLYWGQGTMVTVS

SEQ ID NO: 69 - DNA, 348 bp, Artificial
  Rat Ab 1496 VH region
gaggtgctgctggtggagtctgggggcggctcagtgcagcctgggaggtccatgaaactctcctgtgtagtctcaggattcactttcagtaattatggcatggtctgggtccgccaggctccaaagaagggtctggagtgggtcgcatatattgattctgatggtgataatacttactaccgagattccgtgaagggccgattcactatctccagaaataatgcaaaaagcaccctatatttgcaaatggacagtctgaggtctgaggacacggccacttattactgtacaacagggattgtccggccctttctctattggggccaaggaaccatggtcaccgtctcg

SEQ ID NO: 70 - protein, 14 aa, Artificial
  framework
YFDYWGQGTLVTVS

SEQ ID NO: 71 - protein, 12 aa, Artificial
  framework
YTFGQGTKLEIK

SEQ ID NO: 72 - protein, 447 aa, Artificial
  1519gH20 IgG1 heavy chain (V + human gamma-1 constant)
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 1947 bp, Artificial
  1519gH20 IgG1 heavy chain (V + human gamma-1 constant with exons)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagatctgagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 74 - DNA, 2004 bp, Artificial
  1519gH20 IgG1 heavy chain (V + human gamma-1 constant) with signal sequence
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagatctgagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 75 - DNA, 657 bp, Artificial
  1519 gL20 light chain (V + constant, mammalian expression alternative)
gatatccagatgacccagagcccatctagcttatccgcttccgttggtgatcgcgtgacaattacgtgtaagagctcccaatctctcgtgggtgcaagtggcaagacctatctgtactggctctttcagaagcctggcaaggcaccaaaacggctgatctatctggtgtctacccttgactctgggataccgtcacgattttccggatctgggagcggaactgagttcacactcacgatttcatcgctgcaacccgaggactttgctacctactactgcctgcaaggcactcatttccctcacactttcggccaggggacaaaactcgaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 76 - DNA, 684 bp, Artificial
  1519gH20 Fab′ heavy chain (V + human gamma-1 CH1 + hinge, mammalian expression one base change from SEQ ID NO 38)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcgccgcg

SEQ ID NO: 77 - DNA, 741 bp, Artificial
  1519 gH20 Fab′ heavy chain with signal sequence (mammalian expression one base changed from SEQ ID NO 42)
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcgccgcg

SEQ ID NO: 78 - protein, 346 aa, Artificial
  1519gL20 FabFv light chain (alternative sequence to SEQ ID NO 46)
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 79 - DNA, 1038 bp, Artificial
  1519gL20 FabFv light chain (alternative sequence to SEQ ID NO 47)
gacatccagatgacccagtccccctccagcctgtccgcctccgtgggcgacagagtgaccatcacatgcaagtcctcccagtccctggtcggagcctccggcaagacctacctgtactggctgttccagaagcccggcaaggcccccaagcggctgatctacctggtgtctaccctggactccggcatcccctcccggttctccggctctggctctggcaccgagttcaccctgaccatctccagcctgcagcccgaggacttcgccacctactactgtctgcaaggcacccacttcccccacaccttcggccagggcaccaagctggaaatcaagcggaccgtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggtggaggtggctctggcggtggtggctccggaggcggaggaagcgacatccagatgacccagagcccttcctctgtaagcgccagtgtcggagacagagtgactattacctgccaaagctccccttcagtctggtccaattttctatcctggtatcagcaaaagcccggaaaggctcctaaattgctgatctacgaagcaagcaaactcaccagcggcgtgcccagcaggttcagcggcagtgggtctggaactgactttaccctgacaatctcctcactccagcccgaggacttcgccacctattactgcggtggaggttacagtagcataagtgatacgacatttggatgcggcactaaagtggaaatcaagcgtacc

SEQ ID NO: 80 - DNA, 1071 bp, Artificial
  1519gH20 FabFv heavy chain (alternative sequence to SEQ ID NO 51)
gaggtgcccctggtggaatctggcggcggactggtgcagcctggcggctccctgagactgtcttgcgccgtgtccggcttcaccttctccaactacggcatggtctgggtccgacaggctcctggcaagggactggaatgggtggcctacatcgactccgacggcgacaacacctactaccgggactccgtgaagggccggttcaccatctcccgggacaacgccaagtcctccctgtacctgcagatgaactccctgcgggccgaggacaccgccgtgtactactgcaccaccggcatcgtgcggccctttctgtactggggccagggcaccctggtcaccgtgtcctctgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctctggactctactccctcagcagcgtggtgaccgtgccctccagcagcttgggcacccagacctacatctgcaacgtgaatcacaagcccagcaacaccaaggtggacaagaaagttgagcccaaatcttgttccggaggtggcggttccggaggtggcggtacaggtggcggtgggtccgaagtccagctgcttgaatccggaggcggactcgtgcagcccggaggcagtcttcgcttgtcctgcgctgtatctggaatcgacctgagcaattacgccatcaactgggtgagacaggcacctgggaaatgcctcgaatggatcggcattatatgggctagtgggacgaccttttatgctacatgggcgaagggtagattcacaatctcacgggataatagtaagaacacagtgtacctgcagatgaactccctgcgagcagaggataccgccgtttactattgtgctcgcactgtcccaggttatagcactgcaccctactttgatctgtgggggcagggcactctggtcaccgtctcgtcc

SEQ ID NO: 81 - protein, 112 aa, Artificial
  Rat Ab 1548 VL region (alternative sequence to SEQ ID NO 58)
DVVMTQTPLSLSVAIGQPASISSKSSQSLVGAGGKTYLYWLLQRSGQSPKRLIYLVSTLDSGIPDRFSGSGAETDFTLKIRRVEADDLGVYYCLQGTHFPHTFGAGTNLEIK

SEQ ID NO: 82 - DNA, 336 bp, Artificial
  Rat Ab 1548 VL region (alternative sequence to SEQ ID NO 59)
gatgttgtgatgacccagactccactgtctttgtcggttgccattggacaaccagcctccatctcttctaagtcaagtcagagcctcgtaggtgctggtggaaagacatatttgtattggttattacagaggtccggccagtctccaaagcgactaatctatctggtgtccacactggactctggaattcctgataggttcagtggcagtggagcagagacagattttactcttaaaatccgcagagtggaagccgatgatttgggagtttattactgcttgcaaggtacacattttcctcacacgtttggagctgggaccaacctggaaataaaa

SEQ ID NO: 83 - protein, 116 aa, Artificial
  Rat Ab 1548 VH region (alternative sequence to SEQ ID NO 60)
EVPLVESGGGSVQPGRSMKLSCVVSGFTFSNYGMVWVRQAPKKGLEWVAYIGSDGDNTYYRDSVKGRFTISRNNAKSTLYLQMDSLRSEDTATYYCTTGIVRPFLYWGQGVMVTVS

SEQ ID NO: 84 - DNA, 348 bp, Artificial
  Rat Ab 1548 VH region (alternative sequence to SEQ IS NO 61)
gaggtgccgctggtggagtctgggggcggctcagtgcagcctgggaggtccatgaaactctcctgtgtagtctcaggattcactttcagtaactatggcatggtctgggtccgccaggctccaaagaagggtctggagtgggtcgcatatattggttctgatggtgataatacttactaccgagattccgtgaagggccgattcactatctccagaaataatgcaaaaagcaccctatatttgcaaatggacagtctgaggtctgaggacacggccacttattactgtacaacagggattgtccggccctttctctactggggccaaggagtcatggtcacagtctcg

SEQ ID NO: 85 - DNA, 1947 bp, Artificial
  1519gH20 IgG1 heavy chain (V + human gamma-1 constant with exons one base change to SEQ ID NO 71)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagatctgagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 86 - DNA, 2004 bp, Artificial
  1519gH20 IgG1 heavy chain (V + human gamma-1 constant) with signal sequence (one base change from SEQ ID NO72)
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagatctgagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 87 - protein, 444 aa, Artificial
  1519gH20 IgG4 heavy chain (V + human gamma-4 constant no P mutations)
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - DNA, 1939 bp, Artificial
  1519gH20 IgG4 heavy chain (V + human gamma-4 constant with exons no P mutations)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgcccagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 89 - DNA, 1996 bp, Artificial
  1519gH20 IgG4 heavy chain (V + human gamma-4 constant) with signal sequence - no P mutation
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgcccagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 90 - DNA, 336 bp, Artificial
  1519 gL20 V-region (mammalian expression alternative to SEQ ID NO 17)
gacatccagatgacccagtccccctccagcctgtccgcctccgtgggcgacagagtgaccatcacatgcaagtcctcccagtccctggtcggagcctccggcaagacctacctgtactggctgttccagaagcccggcaaggcccccaagcggctgatctacctggtgtctaccctggactccggcatcccctcccggttctccggctctggctctggcaccgagttcaccctgaccatctccagcctgcagcccgaggacttcgccacctactactgtctgcaaggcacccacttcccccacaccttcggccagggcaccaagctggaaatcaag

SEQ ID NO: 91 - DNA, 657 bp, Artificial
  1519 gL20 light chain (V + constant, mammalian expression) alternative to SEQ ID NO 24)
gacatccagatgacccagtccccctccagcctgtccgcctccgtgggcgacagagtgaccatcacatgcaagtcctcccagtccctggtcggagcctccggcaagacctacctgtactggctgttccagaagcccggcaaggcccccaagcggctgatctacctggtgtctaccctggactccggcatcccctcccggttctccggctctggctctggcaccgagttcaccctgaccatctccagcctgcagcccgaggacttcgccacctactactgtctgcaaggcacccacttcccccacaccttcggccagggcaccaagctggaaatcaagcggaccgtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 92 - DNA, 348 bp, Artificial
  1519 gH20 V-region (mammalian expression alternative to SEQ ID NO 31)
gaggtgcccctggtggaatctggcggcggactggtgcagcctggcggctccctgagactgtcttgcgccgtgtccggcttcaccttctccaactacggcatggtctgggtccgacaggctcctggcaagggactggaatgggtggcctacatcgactccgacggcgacaacacctactaccgggactccgtgaagggccggttcaccatctcccgggacaacgccaagtcctccctgtacctgcagatgaactccctgcgggccgaggacaccgccgtgtactactgcaccaccggcatcgtgcggccctttctgtactggggccagggcaccctggtcaccgtgtcc

SEQ ID NO: 93 - DNA, 1332 bp, Artificial
  1519gH20 IgG4 heavy chain (V + human gamma-4P constant alternative to SEQ ID NO 44)
gaggtgcccctggtggaatctggcggcggactggtgcagcctggcggctccctgagactgtcttgcgccgtgtccggcttcaccttctccaactacggcatggtctgggtccgacaggctcctggcaagggactggaatgggtggcctacatcgactccgacggcgacaacacctactaccgggactccgtgaagggccggttcaccatctcccgggacaacgccaagtcctccctgtacctgcagatgaactccctgcgggccgaggacaccgccgtgtactactgcaccaccggcatcgtgcggccctttctgtactggggccagggcaccctggtcaccgtgtcctctgcctccaccaagggcccctccgtgttccctctggccccttgctcccggtccacctccgagtctaccgccgctctgggctgcctggtcaaggactacttccccgagcccgtgacagtgtcctggaactctggcgccctgacctccggcgtgcacaccttccctgccgtgctgcagtcctccggcctgtactccctgtcctccgtcgtgaccgtgccctcctccagcctgggcaccaagacctacacctgtaacgtggaccacaagccctccaacaccaaggtggacaagcgggtggaatctaagtacggccctccctgccccccctgccctgcccctgaatttctgggcggaccttccgtgttcctgttccccccaaagcccaaggacaccctgatgatctcccggacccccgaagtgacctgcgtggtggtggacgtgtcccaggaagatcccgaggtccagttcaattggtacgtggacggcgtggaagtgcacaatgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgccccctagccaggaagagatgaccaagaaccaggtgtccctgacctgtctggtcaagggcttctacccctccgacattgccgtggaatgggagtccaacggccagcccgagaacaactacaagaccaccccccctgtgctggacagcgacggctccttcttcctgtactctcggctgaccgtggacaagtcccggtggcaggaaggcaacgtcttctcctgctccgtgatgcacgaggccctgcacaaccactacacccagaagtccctgtccctgagcctgggcaag

SEQ ID NO: 94 - protein, 274 aa, Artificial
  FcRn alpha chain extracellular sequence
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSS

SEQ ID NO: 95 - protein, 99 aa, Homo sapiens
IQKTPQIQVYSRHPPENGKPNFLNCYVSQFHPPQIEIELLKNGKKIPNIEMSDLSFSKDWSFYILAHTEFTPTETDVYACRVKHVTLKEPKTVTWDRDM

(19) United States
(12) Patent Application Publication
(10) Pub. No.: US 2022/0144941 A1
(43) Pub. Date: May 12, 2022

(54) METHOD FOR THE TREATMENT OF MYASTHENIA GRAVIS

(71) Applicant: UCB BIOPHARMA SRL, Brussels (BE)

(72) Inventors: Melissa Kaye Brock, Raleigh, NC (US); William James Byrnes, Raleigh, NC (US); Bernhard Heinrich Greve, Monheim Am Rhein (DE); Claus Peter Kiessling, Monheim Am Rhein (DE); Grant Langdon, Hampshire (GB); Elizabeth Webster Manis, Raleigh, NC (US)

(21) Appl. No.: 17/286,247

(22) PCT Filed: Oct. 16, 2019
(86) PCT No.: PCT/EP2019/078093

§ 371 Date: Apr. 16, 2021

Related U.S. Application Data
(60) Provisional application No. 62/746,174, filed on Oct. 16, 2018.

(51) Int. Cl. C07K 16/28; A61K 39/395; A61P 21/04

(52) U.S. Cl. CPC C07K 16/283; A61K 39/3955; A61P 21/04; A61K 2039/545; C07K 2317/51; C07K 2317/515; C07K 2317/565

(57) ABSTRACT

The disclosure relates to a method of treating or preventing Myasthenia Gravis (MG) in a human in need thereof using an anti-FcRn antibody or antigen binding fragment thereof. In particular, the method provides suitable dosage regimens for such treatment.

The disclosure relates to a method of treating myasthenia gravis (MG) using antibodies specific to FcRn.

The neonatal MHC-class-I-like FcRn recycles immunoglobulin and albumin from most cells and transports it bi-directionally across epithelial barriers to affect systemic and mucosal immunity. It was shown that FcRn rescues both IgG and albumin from intracellular lysosomal degradation by recycling it from the sorting endosome to the cell surface (Anderson et al, 2006). With respect to IgG, this is achieved by interaction of IgG with the receptor, FcRn. Thus, in effect FcRn salvages IgG, saving it from degradation and returning it to circulation. Albumin is similarly recycled by FcRn, though via a different binding site on the FcRn molecule. It has been shown that knockout or blockade of FcRn removes this recycling resulting in endosomal catabolism of IgG and a marked reduction of IgG concentrations in both the vascular and extravascular (tissue) compartments. In effect, blockade of FcRn accelerates removal of endogenous IgG and, if the albumin binding site is also blocked, potentially of albumin.

UCB7665 (rozanolixizumab) is a humanized anti-neonatal Fc receptor for IgG (FcRn) monoclonal antibody that has been specifically designed to inhibit IgG binding to FcRn without inhibiting albumin binding to FcRn. UCB7665 is being developed as an inhibitor of FcRn activity with the aim to reduce the concentration of pathogenic IgG in patients with IgG autoantibody mediated diseases.

As individual disease entities, IgG autoantibody mediated conditions are relatively rare. Treatment of these disorders remains a difficult clinical problem, requiring in many of these conditions the long-term use of high-dose corticosteroids alone or combined with cytotoxic agents. These therapeutic approaches are not effective in all patients and conditions and have broad immunosuppressive effects causing considerable toxicity and treatment-related morbidity.

Treatments aimed at reducing the quantity of circulating IgG autoantibody, including plasmapheresis, immunoadsorption, or high-dose intravenous immunoglobulin (IVIg), are being used for primary and secondary therapy of autoimmune disease, particularly where corticosteroid-based immune suppression is not or no longer effective. The therapeutic approach of these treatments is thought to be based on lowering levels of pathogenic autoantibodies, which represents rational and effective treatment modalities of autoimmune diseases.

Myasthenia gravis is a rare autoimmune disorder of the peripheral motor system in which autoimmune antibodies most commonly form against acetylcholine nicotinic receptors (nAChR) at the neuromuscular junction (NMJ). The nAChR autoantibody impairs the ability of acetylcholine to bind to receptors, and leads to the destruction of receptors, either by inducing the muscle cell to eliminate the receptors through endocytosis or by complement fixation.

A second category of MG is due to autoantibodies against the muscle specific kinase (MuSK) protein, a tyrosine kinase receptor which is required for the formation of the NMJ. Antibodies against MuSK inhibit signaling, resulting in a decrease in patency of the NMJ, and the consequent symptoms of MG. In both categories, this results in a characteristic pattern of progressively reduced muscle strength with repeated muscle use and muscle strength recovery following a period of rest. Additional antibodies have been found to be associated with MG but less is yet known about them and they appear less common than the main two.

The essential role of the autoimmune antibodies in mediating this pathology is supported by the improvement seen after plasma exchange (PLEX). Plasma Exchange which reduces IgG levels including pathogenic IgG autoantibody is used both in patients non-responsive to acetylcholinesterase (AChE) inhibitors or immunosuppressive treatments and in patients experiencing myasthenic crisis (Gilhus and Verschuuren, 2015).

Although the prognosis of MG has markedly improved over the last decades, with new therapies dramatically improving survival, significant mortality and even morbidity remains an issue. Treatment of MG remains a difficult clinical problem, requiring the long-term use of high-dose corticosteroids alone or combined with cytotoxic agents. Many of the therapies thought to be effective in MG have insufficient data to clearly support their use, are not effective in all patients and conditions, and have broad immunosuppressive effects causing considerable toxicity and treatment-related morbidity. Moreover, due to the natural fluctuations in the course of the disease, many patients need an effective treatment for acute situations requiring urgent treatment.

Both PLEX and IVIg currently are used as the standard of care to improve symptoms in situations requiring chronic-intermittent treatment; however, neither treatment is approved in the US for MG, and the procedures often are burdensome for the patients. Thus a significant unmet medical need exists in this patient population for an effective chronic-intermittent treatment with increased convenience for patients with generalized MG.

There thus remains a considerable unmet medical need for new therapeutic options in the treatment of MG.

Accordingly agents that block or reduce the binding of IgG to FcRn may be useful in the treatment or prevention of MG by removal of pathogenic IgG. Anti-FcRn antibodies have been described previously in WO2009/131702, WO2007/087289, WO2006/118772, WO2014/019727, WO2015/071330, WO2015/167293 and WO2016/123521.

UCB7665 (rozanolixizumab) is a humanized anti-FcRn monoclonal antibody that has been specifically designed to inhibit IgG binding to FcRn without inhibiting albumin binding to FcRn (described herein and in WO2014/019727 and in Smith et al., 2019, MABS, 10, 111-1130). UCB7665 is being developed as an inhibitor of FcRn activity with the aim to reduce the concentration of pathogenic IgG in patients with MG. UCB7665 was derived from a rat antibody with specificity for human FcRn by engineering the rat antibody into a humanized IgG4P format. The construct encoding UCB7665 was created by grafting the complementarity-determining region (CDRs) from the parental rat heavy and light chain variable regions onto a human IgG4P and kappa chain genetic background (SEQ ID NO:43 and SEQ ID NO:22 respectively).

Kiessling et al 2017, Sci. Transl. Med. 9, pgs. 1-12 describes a randomized, subject-blind, investigator-blind, placebo-controlled, single dose-escalating phase 1 clinical trial of rozanolixizumab in healthy subjects. The antibody reduced serum IgG, without any statistically significant reduction in serum albumin concentration. Acceptable safety, pharmacokinetic and pharmacodynamic profiles, were obtained.

### SUMMARY OF THE DISCLOSURE

The present disclosure demonstrates for the first time, the therapeutic efficacy of anti-FcRn antibodies in the treatment of MG in humans and provides suitable dosage regimens for such treatment.

Thus in one aspect there is provided a method of treating or preventing myasthenia gravis (MG) in a human in need thereof, the method comprising administering to the human at least 3 doses of an anti-FcRn antibody or an antigen binding fragment thereof wherein each dose is independently selected from 4 mg/kg, 7 mg/kg, 10 mg/kg, 15 mg/kg and 20 mg/kg.

In one aspect fixed unit dosing is used, optionally over body weight tiers. In one example a fixed unit dose equivalent to approximately 7 mg/kg is used. Accordingly, in one example the present invention also provides a method of treating or preventing myasthenia gravis (MG) in a human in need thereof, the method comprising administering to the human at least 3 doses, preferably at least 6 doses, of an anti-FcRn antibody or antigen-binding fragment thereof wherein for a body weight of less than 50 kg the dose is 280 mg, for a body weight of equal to or greater than 50 kg but less than 70 kg the dose is 420 mg, for a body weight of equal to or greater than 70 kg but less than 100 kg the dose is 560 mg and for a body weight of equal to or greater than 100 kg the dose is 840 mg.

In one example a fixed unit dose equivalent to approximately 10 mg/kg is used. Accordingly, in one example the present invention also provides a method of treating or preventing myasthenia gravis (MG) in a human in need thereof, the method comprising administering to the human at least 3 doses, preferably at least 6 dose of an anti-FcRn antibody or antigen-binding fragment thereof wherein for a body weight of less than 50 kg the dose is 420 mg, for a body weight of equal to or greater than 50 kg but less than 70 kg the dose is 560 mg, for a body weight of equal to or greater than 70 kg but less than 100 kg the dose is 840 mg and for a body weight of equal to or greater than 100 kg the dose is 1120 mg.

In one example, the anti-FcRn antibody or antigen binding fragment thereof comprises:
a. a heavy chain or heavy chain fragment having a variable region, wherein said variable region comprises three CDRs having the sequences given in SEQ ID NO: 1 for CDR H1, SEQ ID NO: 2 for CDR H2 and SEQ ID NO: 3 for CDR H3, and
b. a light chain or light chain fragment having a variable region, wherein said variable region comprises three CDRs having the sequences given in SEQ ID NO: 4 for CDR L1, SEQ ID NO: 5 for CDR L2 and SEQ ID NO: 6 for CDR L3.

In another aspect, there is provided an anti-FcRn antibody or antigen binding fragment thereof for use in the treatment or prevention of myasthenia gravis (MG) in a human in need thereof, comprising administering to the human at least 3 doses of an anti-FcRn antibody or an antigen binding fragment thereof wherein each dose is independently selected from 4 mg/kg, 7 mg/kg, 10 mg/kg, 15 mg/kg and 20 mg/kg.

In another aspect there is provided an anti-FcRn antibody or an antigen binding fragment thereof comprising:

a heavy chain or heavy chain fragment having a variable region, wherein said variable region comprises three CDRs having the sequences given in SEQ ID NO: 1 for CDR H1, SEQ ID NO: 2 for CDR H2 and SEQ ID NO: 3 for CDR H3, and
a light chain or light chain fragment having a variable region wherein said variable region comprises three CDRs having the sequences given in SEQ ID NO: 4 for CDR L1, SEQ ID NO: 5 for CDR L2 and SEQ ID NO: 6 for CDR L3,
for use in the treatment or prevention of myasthenia gravis (MG) in a human in need thereof comprising administering to the human at least 3 doses of an anti-FcRn antibody or antigen binding fragment thereof wherein each dose is independently selected from 4 mg/kg, 7 mg/kg, 10 mg/kg, 15 mg/kg and 20 mg/kg.

In another aspect there is provided the use of an anti-FcRn antibody or binding fragment thereof comprising:
i. a heavy chain or heavy chain fragment having a variable region, wherein said variable region comprises three CDRs having the sequences given in SEQ ID NO: 1 for CDR H1, SEQ ID NO: 2 for CDR H2 and SEQ ID NO: 3 for CDR H3, and
ii. a light chain or light chain fragment thereof having a variable region comprising three CDRs having the sequences given in SEQ ID NO: 4 for CDR L1, SEQ ID NO: 5
   for CDR L2 and SEQ ID NO: 6 for CDR L3, for the manufacture of a medicament for the treatment or prevention of myasthenia grays (MG) comprising administering to the human at least 3 doses of an anti-FcRn antibody or FcRn binding fragment thereof wherein each dose is independently selected from 4 mg/kg, 7 mg/kg, 10 mg/kg, 15 mg/kg and 20 mg/kg.

Importantly the antibodies of the present invention are able to bind human FcRn at both pH6 and pH7.4 with comparable and high binding affinity. Advantageously therefore the antibodies are able to continue to bind FcRn even within the endosome, thereby maximising the blocking of FcRn binding to IgG.

In one example, the anti-FcRn antibody or binding fragment thereof for use in the present invention binds an epitope of human FcRn which comprises at least one amino acid selected from the group consisting of residues V105, P106, T107, A108 and K109 of SEQ ID NO:94 and at least one residue, for example at least 2, 3, 4, 5, 6, 7, 8, 9 or 10 residues selected from the group consisting of P100, E115, E116, F117, M118, N119, F120, D121, L122, K123, Q124, G128, G129, D130, W131, P132 and E133 of SEQ ID NO:94

In one embodiment the antibodies or binding fragments according to the present disclosure comprise a heavy chain or heavy chain fragment having a variable region, for example comprising one, two or three CDRs independently selected from SEQ ID NO: 1, SEQ ID NO: 2 and SEQ ID NO: 3, in particular wherein CDR H1 is SEQ ID NO: 1, CDR H2 is SEQ ID NO: 2 and CDR H3 is SEQ ID NO: 3.

In one embodiment the antibodies or binding fragments according to the present disclosure comprise a light chain or light chain fragment having a variable region, for example comprising one, two or three CDRs independently selected from SEQ ID NO: 4, SEQ ID NO: 5 and SEQ ID NO: 6, in particular wherein CDR L1 is SEQ ID NO: 4, CDR L2 is SEQ ID NO: 5 and CDR L3 is SEQ ID NO: 6.

In one embodiment the antibodies or binding fragments according to the present disclosure comprise CDR sequences of SEQ ID NOs: 1 to 6, for example wherein CDR H1 is SEQ ID NO: 1, CDR H2 is SEQ ID NO: 2, CDR H3 is SEQ ID NO: 3, CDR L1 is SEQ ID NO: 4, CDR L2 is SEQ ID NO: 5 and CDR L3 is SEQ ID NO: 6.

The present disclosure also relates to pharmaceutical compositions comprising said antibodies and fragments.

### BRIEF DESCRIPTION OF THE FIGURES

FIG. 1 shows certain amino acid and polynucleotide sequences.

FIG. 2 MG0002 study design (SubQ; UCB7665)

FIG. 3 Change from baseline in MG-ADL score

FIG. 4 Change from baseline in QMG, MG-Composite, MG-ADL scores, serum IgG concentration and anti-AChR antibody (rozanolixizumab 7 mg/kg/rozanolixizumab 7 mg/kg)

FIG. 5 Quantitative Myasthenia Gravis testing form

FIG. 6 Myasthenia Gravis Composite Score

FIG. 7 Myasthenia Gravis Activities of Daily Living (MG-ADL) Scoring

### DETAILS OF THE DISCLOSURE

Myasthenia gravis (MG) is a debilitating and potentially fatal autoimmune disease characterized by autoantibodies directed against epitopes of the post-synpatic muscle membrane, including the nicotinic acetylcholine receptor (AChR) and the muscle-specific tyrosine kinase receptor (MuSK), and complement-mediated destruction of the post-junctional membrane. Clinical manifestations include fluctuating weakness of ocular, bulbar, respiratory and limb muscles. Current long-term therapies for MG include thymectomy (THX), cholinesterase inhibitors, and immunosuppressive or immunomodulatory agents. Exacerbations are typically treated with therapies such as intravenous or subcutaneous immunoglobulins (IVIg or SCIg) and plasma exchange (PLEX).

In most patients the first muscles to be affected are those controlling eye and eyelid movements. In some patients, the Myasthenia Gravis only ever involves the eye muscles (ocular Myasthenia Gravis) while in the majority there is also involvement of other muscles (generalised Myasthenia Gravis). Generalised Myasthenia Gravis as used herein therefore refers to myasthenia gravis which affects other muscles beyond just ocular muscles.

In one aspect the present invention provides a method of treating or preventing myasthenia gravis (MG) in a human in need thereof, the method comprising administering to the human at least 6 doses of an anti-FcRn antibody or an antigen binding fragment thereof wherein each dose is independently selected from 4 mg/kg, 7 mg/kg, 10 mg/kg, 15 mg/kg and 20 mg/kg, preferably 7 mg/kg or 10 mg/kg.

In one example fixed unit dosing may be used, optionally using weight based tiers as described herein below.

Any suitable anti-FcRn antibody or antigen binding fragment thereof may be used in the present invention, including those described herein.

In one example, the anti-FcRn antibody or antigen binding fragment thereof comprises:
a. a heavy chain or heavy chain fragment having a variable region, wherein said variable region comprises three CDRs having the sequences given in SEQ ID NO: 1 for CDR H1, SEQ ID NO: 2 for CDR H2 and SEQ ID NO: 3 for CDR H3, and
b. a light chain or light chain fragment having a variable region, wherein said variable region comprises three CDRs having the sequences given in SEQ ID NO: 4 for CDR L1, SEQ ID NO: 5 for CDR L2 and SEQ ID NO: 6 for CDR L3.

FcRn as employed herein refers to the non-covalent complex between the human IgG receptor alpha chain, also known as the neonatal Fc receptor, the amino acid sequence of which is in UniProt under number P55899 together with β2 microglobulin (β2M), the amino acid sequence of which is in UniProt under number P61769.

Antibody molecule as employed herein refers to an antibody or antigen binding fragment.

The term ‘antibody’ as used herein generally relates to intact (whole) antibodies i.e. comprising the elements of two full length heavy chains and light chains. The antibody may comprise further additional binding domains for example as per the molecule DVD-Ig as disclosed in WO 2007/024715, or the so-called (FabFv)₂Fc described in WO2011/030107. Thus antibody as employed herein includes bi, tri or tetra-valent full length antibodies.

As described herein above, the antibody for use in the method comprises a complete antibody molecule having full length heavy and light chains. Alternatively, the method employs an antigen binding fragment. Antigen-binding fragments may include a conventional antibody fragment structure, e.g., a Fab fragment, modified Fab, Fab′, or a F(ab′)2 fragment. An antibody can be cleaved into fragments by enzymes, such as, e.g., papain (to produce two Fab fragments and an Fc fragment) and pepsin (to produce a F(ab′)2 fragment and a pFc′ fragment). The antigen-binding fragment may also comprise a non-conventional structure (i.e., comprise antigen-binding portions of an antibody in an alternative format, which include polypeptides that mimic antibody fragment activity by retaining antigen-binding capacity).

In this regard, antigen-binding fragment includes domain antibodies or nanobodies, e.g., VH, VL, V_{HH}, and V_{NAR}-based structures, single chain antibodies (scFv), peptibody or peptide-Fc fusion, as well as di- and multimeric antibody-like molecules like dia-, tria- and tetra-bodies, or minibodies (miniAbs) that comprise different formats consisting of scFvs linked to oligomerization domains. Examples of multi-specific antigen-binding fragments include Fab-Fv, Fab-dsFv, Fab-Fv-Fv, Fab-scFv-scFv, Fab-Fv-Fc and Fab-dsFv-PEG fragments described in International Patent Application Publication Nos. WO2009040562, WO2010035012, WO2011/08609, WO2011/030107 and WO2011/061492, respectively, all of which are hereby incorporated by reference with respect to their discussion of antigen-binding moieties. A further example of multi-specific antigen-binding fragments include V_{HH} fragments linked in series. An alternative antigen-binding fragment comprises a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin). Such antibody fragments are described in International Patent Application Publication No, WO2015/197772, which is hereby incorporated by reference in its entirety and particularly with respect to the discussion of antibody fragments. Antibody fragments and methods of producing them are well known in the art, see for example Verma et al., 1998, Journal of Immunological Methods, 216, 165-181; Adair and Lawson, 2005. Therapeutic antibodies. Drug Design Reviews—Online 2(3):209-217. Examples of multi-specific antibodies or antigen-binding fragments thereof, which also are contemplated for use in the context of the disclosure, include bi, tri or tetra-valent antibodies, Bis-scFv, diabodies, triabodies, tetrabodies, bibodies and tribodies (see for example Holliger and Hudson, 2005, Nature Biotech 23(9): 1126-1136; Schoonjans et al. 2001, Biomolecular Engineering, 17(6), 193-202).

Disclosure herein relating to antibodies, particularly with respect to epitopes, binding affinity and specificity, and activity, also is applicable to antibody fragments and antibody-like molecules. It will be appreciated that antibody fragments also may be characterized as monoclonal, chimeric, humanized, fully human, multi-specific, bi-specific etc., and that discussion of these terms below also relate to antigen-binding fragments.

The above antibodies and antigen-binding fragments are described for purposes of reference and example only and do not limit the scope of invention.

In one embodiment the antibody or antigen binding fragment comprises a binding domain. A binding domain will generally comprise 6 CDRs, three from a heavy chain and three from a light chain. In one embodiment the CDRs are in a framework and together form a variable region. Thus in one embodiment an antibody or antigen-binding fragment comprises a binding domain specific for antigen comprising a light chain variable region and a heavy chain variable region.

It will be appreciated that one or more (for example 1, 2, 3 or 4) amino acid substitutions, additions and/or deletions may be made to the CDRs or other sequences (e.g variable domains) provided by the present invention without significantly altering the ability of the antibody to bind to FcRn. The effect of any amino acid substitutions, additions and/or deletions can be readily tested by one skilled in the art, for example by using the methods described in WO2014/019727 to determine FcRn binding and blocking.

In one example one or more (for example 1, 2, 3 or 4) amino acid substitutions, additions and/or deletions may be made to the framework region employed in the antibody or fragment provided by the present invention and wherein binding affinity to FcRn is retained or increased.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al. (supra)”). This numbering system is used in the present specification except where otherwise indicated.

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.

The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus unless indicated otherwise ‘CDR-H1’ as employed herein is intended to refer to residues 26 to 35, as described by a combination of the Kabat numbering system and Chothia's topological loop definition.

The CDRs of the light chain variable domain are located at residues 24-34 (CDR-L1), residues 50-56 (CDR-L2) and residues 89-97 (CDR-L3) according to the Kabat numbering system.

Antibodies and antigen-binding fragments of the present disclosure block FcRn and may thereby prevent it functioning in the recycling of IgG. Blocking as employed herein refers to physically blocking such as occluding the receptor but will also include where the antibody or fragments binds an epitope that causes, for example a conformational change which means that the natural ligand to the receptor no longer binds. Antibody molecules of the present invention bind to FcRn and thereby decrease or prevent (e.g. inhibit) FcRn binding to an IgG constant region.

In one embodiment the antibody or antigen-binding fragment binds FcRn competitively with respect to IgG.

In one example the antibody or antigen-binding fragment functions as a competitive inhibitor of human FcRn binding to human IgG. In one example the antibody or antigen-binding fragment binds to the IgG binding site on FcRn. In one example the antibody or antigen-binding fragment does not bind β2M.

Antibodies for use in the present disclosure may be obtained using any suitable method known in the art. The FcRn polypeptide/protein including fusion proteins, cells (recombinantly or naturally) expressing the polypeptide (such as activated T cells) can be used to produce antibodies which specifically recognise FcRn. The polypeptide may be the ‘mature’ polypeptide or a biologically active fragment or derivative thereof. The human protein is registered in Swiss-Prot under the number P55899. The extracellular domain of human FcRn alpha chain is provided in SEQ ID NO:94. The sequence of β2M is provided in SEQ ID NO:95.

In one embodiment the antigen is a mutant form of FcRn which is engineered to present FcRn on the surface of a cell, such that there is little or no dynamic processing where the FcRn is internalised in the cell, for example this can be achieved by making a mutation in the cytoplasmic tail of the FcRn alpha chain, wherein di-leucine is mutated to di-alanine as described in Ober et al 2001 Int. Immunol. 13, 1551-1559.

Polypeptides, for use to immunize a host, may be prepared by processes well known in the art from genetically engineered host cells comprising expression systems or they may be recovered from natural biological sources. In the present application, the term “polypeptides” includes peptides, polypeptides and proteins. These are used interchangeably unless otherwise specified. The FcRn polypeptide may in some instances be part of a larger protein such as a fusion protein for example fused to an affinity tag or similar.

Antibodies generated against the FcRn polypeptide may be obtained, where immunisation of an animal is necessary, by administering the polypeptides to an animal, preferably a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels or pigs may be immunized. However, mice, rabbits, pigs and rats are generally most suitable.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Antibodies for use in the invention may also be generated using single lymphocyte antibody methods by cloning and expressing immunoglobulin variable region cDNAs generated from single lymphocytes selected for the production of specific antibodies by, for example, the methods described by Babcook, J. et al., 1996, Proc. Natl. Acad. Sci. USA 93(15):7843-78481; WO92/02551; WO2004/051268 and International Patent Application number WO2004/106377.

Screening for antibodies can be performed using assays to measure binding to human FcRn and/or assays to measure the ability to block IgG binding to the receptor. An example of a binding assay is an ELISA, in particular, using a fusion protein of human FcRn and human Fc, which is immobilized on plates, and employing a secondary antibody to detect anti-FcRn antibody bound to the fusion protein. Examples of suitable antagonistic and blocking assays are well known in the art and described in WO2014/019727.

Specific as employed herein is intended to refer to an antibody that only recognises the antigen to which it is specific or an antibody that has significantly higher binding affinity to the antigen to which it is specific compared to binding to antigens to which it is non-specific, for example at least 5, 6, 7, 8, 9, 10 times higher binding affinity. Binding affinity may be measured by techniques such as BIAcore as described herein and in WO2014/019727. In one example the antibody of the present invention does not bind β2 microglobulin (β2M). In one example the antibody of the present invention binds cynomolgus FcRn. In one example the antibody of the present invention does not bind rat or mouse FcRn.

The amino acid sequences and the polynucleotide sequences of certain antibodies according to the present disclosure are provided in the Figures.

Other antibodies useful in the present invention are described in WO2009/131702, WO2007/087289, WO2006/118772, WO2015/071330, WO2015/167293 and

WO2016/123521 and are incorporated herein by reference. Examples also include M281 from Momenta Pharmaceuticals and SYNT0001 from Syntimmune.

In one embodiment the antibody or fragments according to the disclosure are humanised.

As used herein, the term ‘humanised antibody molecule’ refers to an antibody molecule wherein the heavy and/or light chain contains one or more CDRs (including, if desired, one or more modified CDRs) from a donor antibody (e.g. a non-human antibody such as a murine monoclonal antibody) grafted into a heavy and/or light chain variable region framework of an acceptor antibody (e.g. a human antibody). For a review, see Vaughan et al, Nature Biotechnology, 16, 535-539, 1998. In one embodiment rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein above are transferred to the human antibody framework. In another embodiment only the specificity determining residues from each of the CDRs described herein above are transferred to the human antibody framework.

When the CDRs or specificity determining residues are grafted, any appropriate acceptor variable region framework sequence may be used having regard to the class/type of the donor antibody from which the CDRs are derived, including mouse, primate and human framework regions.

Suitably, the humanised antibody according to the present invention has a variable domain comprising human acceptor framework regions as well as one or more of the CDRs provided specifically herein. Thus, provided in one embodiment is blocking humanised antibody which binds human FcRn wherein the variable domain comprises human acceptor framework regions and non-human donor CDRs.

Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/In a humanised antibody of the present invention, the acceptor heavy and light chains do not necessarily need to be derived from the same antibody and may, if desired, comprise composite chains having framework regions derived from different chains.

One such suitable framework region for the heavy chain of the humanised antibody of the present invention is derived from the human sub-group VH3 sequence 1-3 3-07 together with JH4 (SEQ ID NO: 56).

Accordingly, in one example there is provided a humanised antibody comprising the sequence given in SEQ ID NO: 1 for CDR-H1, the sequence given in SEQ ID NO: 2 for CDR-H2 and the sequence given in SEQ ID NO: 3 for CDRH3, wherein the heavy chain framework region is derived from the human subgroup VH3 sequence 1-3 3-07 together with JH4.

The sequence of human JH4 is as follows: (YFDY)WGQGTLVTVS (Seq ID No: 70). The YFDY motif is part of CDR-H3 and is not part of framework 4 (Ravetch, J V. et al., 1981, Cell, 27, 583-591).

In one example the heavy chain variable domain of the antibody comprises the sequence given in SEQ ID NO: 29.

A suitable framework region for the light chain of the humanised antibody of the present invention is derived from the human germline sub-group VK1 sequence 2-1-(1) A30 together with JK2 (SEQ ID NO: 54).

Accordingly, in one example there is provided a humanised antibody comprising the sequence given in SEQ ID NO: 4 for CDR-L1, the sequence given in SEQ ID NO: 5 for CDR-L2 and the sequence given in SEQ ID NO: 6 for CDRL3, wherein the light chain framework region is derived from the human subgroup VK1 sequence 2-1-(1) A30 together with JK2.

The JK2 sequence is as follows: (YT)FGQGTKLEIK (Seq ID No: 71). The YT motif is part of CDR-L3 and is not part of framework 4 (Hieter, P A., et al., 1982, J. Biol. Chem., 257, 1516-1522).

In one example the light chain variable domain of the antibody comprises the sequence given in SEQ ID NO: 15.

In a humanised antibody of the present invention, the framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

Thus in one embodiment 1, 2, 3, 4, or 5 residues in the framework are replaced with an alternative amino acid residue.

Accordingly, in one example there is provided a humanised antibody, wherein at least the residues at each of positions 3, 24, 76, 93 and 94 of the variable domain of the heavy chain (Kabat numbering) are donor residues, see for example the sequence given in SEQ ID NO: 29.

In one embodiment residue 3 of the heavy chain variable domain is replaced with an alternative amino acid, for example glutamine.

In one embodiment residue 24 of the heavy chain variable domain is replaced with an alternative amino acid, for example alanine.

In one embodiment residue 76 of the heavy chain variable domain is replaced with an alternative amino acid, for example asparagine.

In one embodiment residue 93 of the heavy chain is replaced with an alternative amino acid, for example alanine.

In one embodiment residue 94 of the heavy chain is replaced with an alternative amino acid, for example arginine.

In one embodiment residue 3 is glutamine, residue 24 is alanine, residue 76 is aspargine, residue 93 is alanine and residue 94 is arginine in the humanised heavy chain variable region according to the present disclosure.

Accordingly, in one example there is provided a humanised antibody, wherein at least the residues at each of positions 36, 37 and 58 of the variable domain of the light chain (Kabat numbering) are donor residues, see for example the sequence given in SEQ ID NO: 15

In one embodiment residue 36 of the light chain variable domain is replaced with an alternative amino acid, for example tyrosine.

In one embodiment residue 37 of the light chain variable domain is replaced with an alternative amino acid, for example glutamine.

In one embodiment residue 58 of the light chain variable domain is replaced with an alternative amino acid, for example valine.

In one embodiment residue 36 is tyrosine, residue 37 is glutamine and residue 58 is valine, in the humanised heavy chain variable region according to the present disclosure.

In one embodiment the disclosure provides an antibody sequence which is 80% similar or identical to a sequence disclosed herein, for example 85%, 90%, 91%, 92%, 93%, 94%, 95% 96%, 97%, 98% or 99% over part or whole of the relevant sequence, for example a variable domain sequence, a CDR sequence or a variable domain sequence, excluding the CDRs. In one embodiment the relevant sequence is SEQ ID NO: 15. In one embodiment the relevant sequence is SEQ ID NO: 29.

In one embodiment, the present invention provides an antibody molecule which binds human FcRn comprising a heavy chain, wherein the variable domain of the heavy chain comprises a sequence having at least 80%, 85%, 90%, 91%, 92%, 93%, 94%, 95% 96%, 97%, 98% or 99% identity or similarity to the sequence given in SEQ ID NO:29.

In one embodiment, the present invention provides an antibody molecule which binds human FcRn comprising a light chain, wherein the variable domain of the light chain comprises a sequence having at least 80%, 85%, 90%, 91%, 92%, 93%, 94%, 95% 96%, 97%, 98% or 99% identity or similarity to the sequence given in SEQ ID NO:15.

In one embodiment the present invention provides an antibody molecule which binds human FcRn wherein the antibody has a heavy chain variable domain which is at least 90%, 91%, 92%, 93%, 94%, 95% 96%, 97%, 98% or 99% similar or identical to the sequence given in SEQ ID NO:29 but wherein the antibody molecule has the sequence given in SEQ ID NO: 1 for CDR-H1, the sequence given in SEQ ID NO: 2 for CDR-H2 and the sequence given in SEQ ID NO: 3 for CDR-H3.

In one embodiment the present invention provides an antibody molecule which binds human FcRn wherein the antibody has a light chain variable domain which is at least 90%, 91%, 92%, 93%, 94%, 95% 96%, 97%, 98% or 99% similar or identical to the sequence given in SEQ ID NO:15 but wherein the antibody molecule has the sequence given in SEQ ID NO: 4 for CDR-L1, the sequence given in SEQ ID NO: 5 for CDR-L2 and the sequence given in SEQ ID NO:6 for CDR-L3.

In one embodiment the present invention provides an antibody molecule which binds human FcRn wherein the antibody has a heavy chain variable domain which is at least 90%, 91%, 92%, 93%, 94%, 95% 96%, 97%, 98% or 99% similar or identical to the sequence given in SEQ ID NO:29 and a light chain variable domain which is at least 90%, 91%, 92%, 93%, 94%, 95% 96%, 97%, 98% or 99% similar or identical to the sequence given in SEQ ID NO:15 but wherein the antibody molecule has the sequence given in SEQ ID NO: 1 for CDR-H1, the sequence given in SEQ ID NO: 2 for CDR-H2, the sequence given in SEQ ID NO: 3 for CDR-H3, the sequence given in SEQ ID NO: 4 for CDR-L1, the sequence given in SEQ ID NO: 5 for CDR-L2 and the sequence given in SEQ ID NO:6 for CDR-L3.

“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains). Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656).

The antibody molecules of the present invention may comprise a complete antibody molecule having full length heavy and light chains or a fragment thereof and may be, but are not limited to Fab, modified Fab, Fab′, modified Fab′, F(ab′)₂, Fv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody fragments are well known in the art (see for example Verma et al., 1998, Journal of Immunological Methods, 216, 165-181). Other antibody fragments for use in the present invention include the Fab and Fab′ fragments described in International patent applications WO2005/003169, WO2005/003170 and WO2005/003171. Multi-valent antibodies may comprise multiple specificities e.g bispecific or may be monospecific (see for example WO 92/22853, WO05/113605, WO2009/040562 and WO2010/035012).

In one embodiment the antibody molecule of the present disclosure is an antibody Fab′ fragment comprising the variable regions shown in SEQ ID NOs: 15 and 29 for example for the light and heavy chain respectively. In one embodiment the antibody molecule has a light chain comprising the sequence given in SEQ ID NO:22 and a heavy chain comprising the sequence given in SEQ ID NO:36.

In one embodiment the antibody molecule of the present disclosure is a full length IgG1 antibody comprising the variable regions shown in SEQ ID NOs: 15 and 29 for example for the light and heavy chain respectively. In one embodiment the antibody molecule has a light chain comprising the sequence given in SEQ ID NO:22 and a heavy chain comprising the sequence given in SEQ ID NO:72.

In one embodiment the antibody molecule of the present disclosure is a full length IgG4 format comprising the variable regions shown in SEQ ID NOs: 15 and 29 for example for the light and heavy chain respectively. In one embodiment the antibody molecule has a light chain comprising the sequence given in SEQ ID NO:22 and a heavy chain comprising the sequence given in SEQ ID NO:87.

In one embodiment the antibody molecule of the present disclosure is a full length IgG4P format comprising the variable regions shown in SEQ ID NOs: 15 and 29 for example for the light and heavy chain respectively. In one embodiment the antibody molecule has a light chain comprising the sequence given in SEQ ID NO:22 and a heavy chain comprising the sequence given in SEQ ID NO:43.

IgG4P as employed herein is a mutation of the wild-type IgG4 isotype where amino acid 241 is replaced by proline see for example where serine at position 241 has been changed to proline as described in Angal et al., Molecular Immunology, 1993, 30 (1), 105-108.

In one embodiment the antibody according to the present disclosure is provided as an FcRn binding antibody fusion protein which comprises an immunoglobulin moiety, for example a Fab or Fab′ fragment, and one or two single domain antibodies (dAb) linked directly or indirectly thereto, for example as described in WO2009/040562, WO2010035012, WO2011/030107, WO2011/061492 and WO2011/086091 all incorporated herein by reference.

In one embodiment the fusion protein comprises two domain antibodies, for example as a variable heavy (VH) and variable light (VL) pairing, optionally linked by a disulphide bond.

In one embodiment the Fab or Fab′ element of the fusion protein has the same or similar specificity to the single domain antibody or antibodies. In one embodiment the Fab or Fab′ has a different specificity to the single domain antibody or antibodies, that is to say the fusion protein is multivalent. In one embodiment a multivalent fusion protein according to the present invention has an albumin binding site, for example a VH/VL pair therein provides an albumin binding site. In one such embodiment the heavy chain comprises the sequence given in SEQ ID NO:50 and the light chain comprises the sequence given in SEQ ID NO:46 or SEQ ID NO:78.

In one embodiment the Fab or Fab′ according to the present disclosure is conjugated to a PEG molecule or human serum albumin.

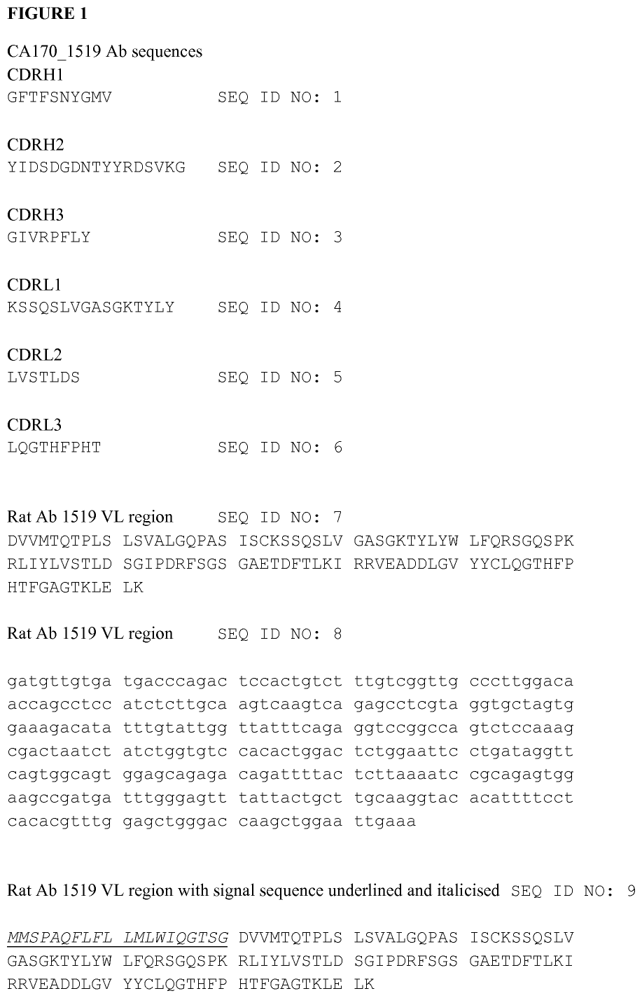

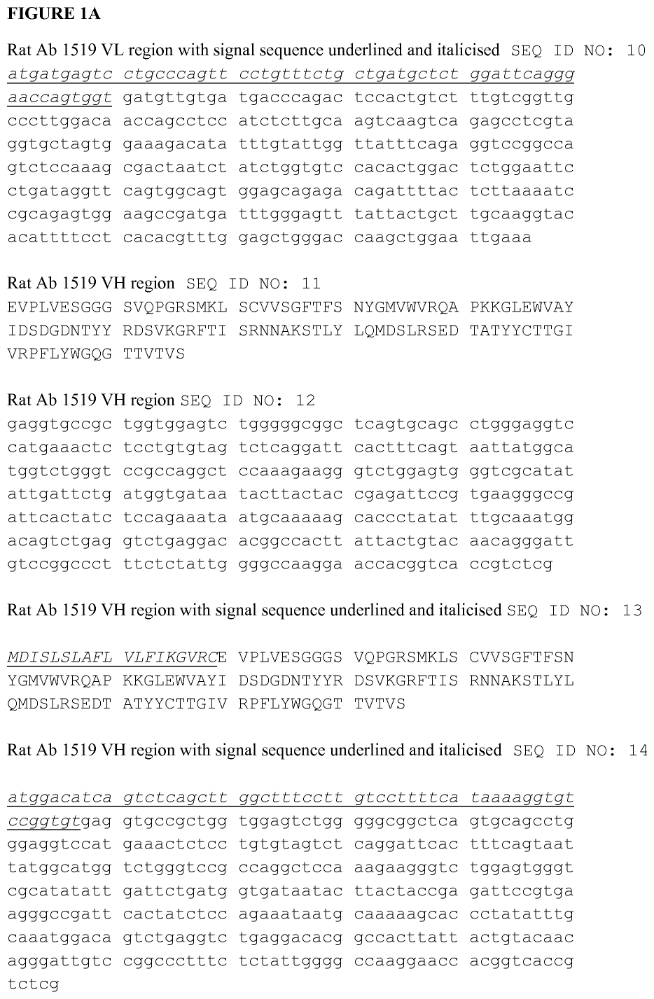

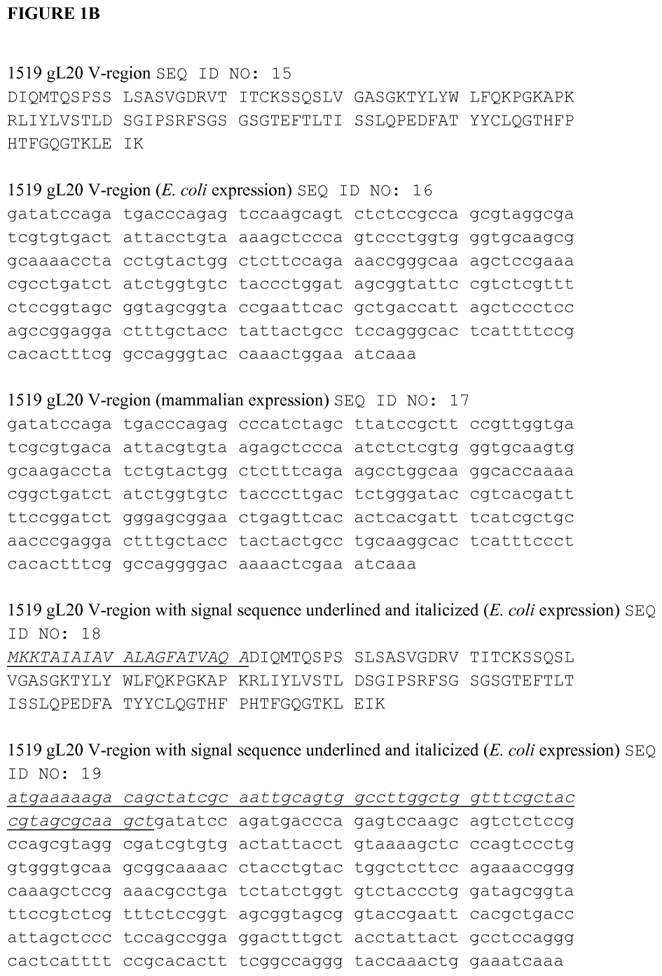

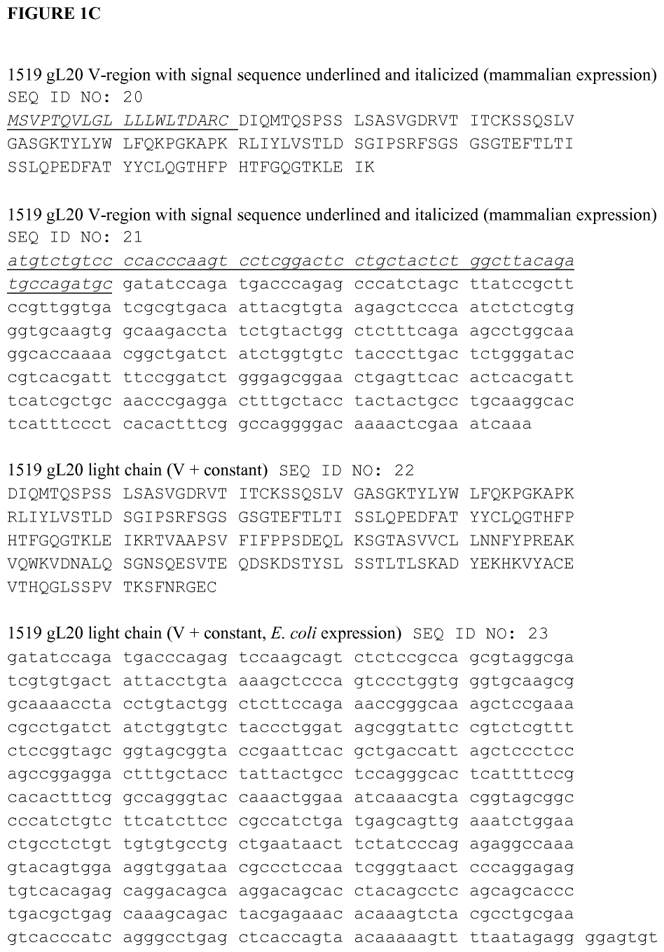

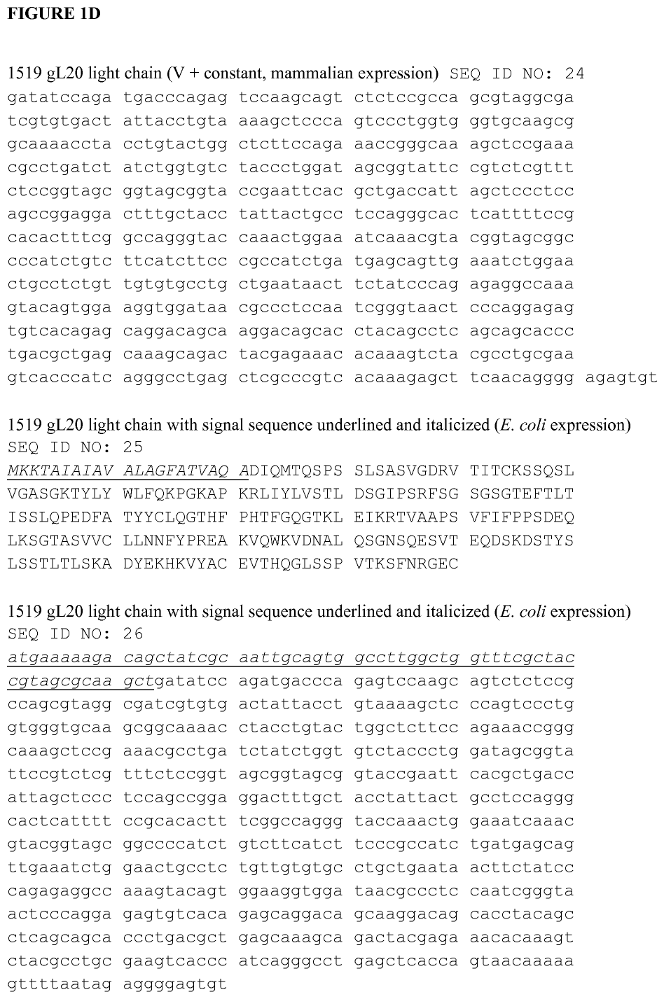

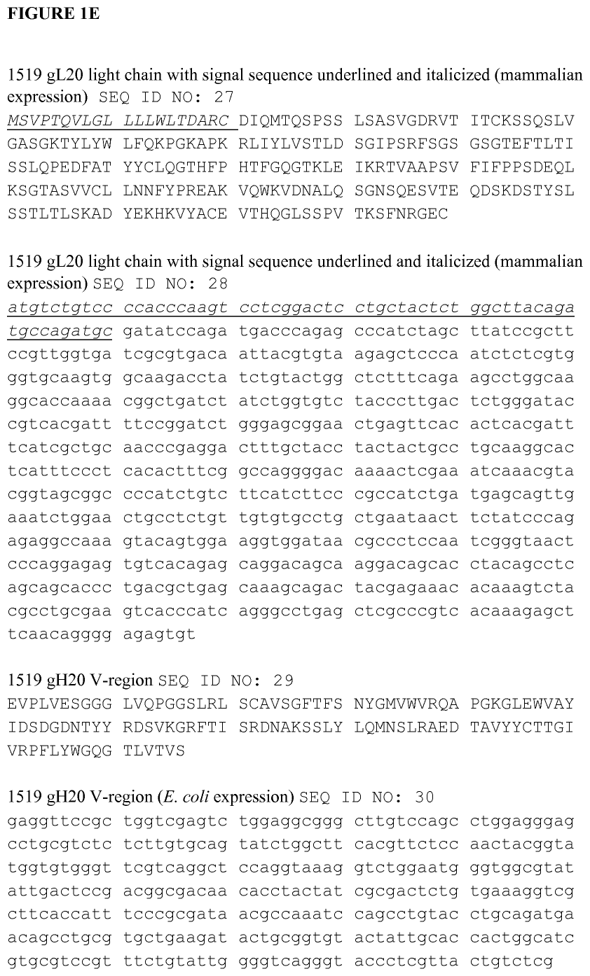

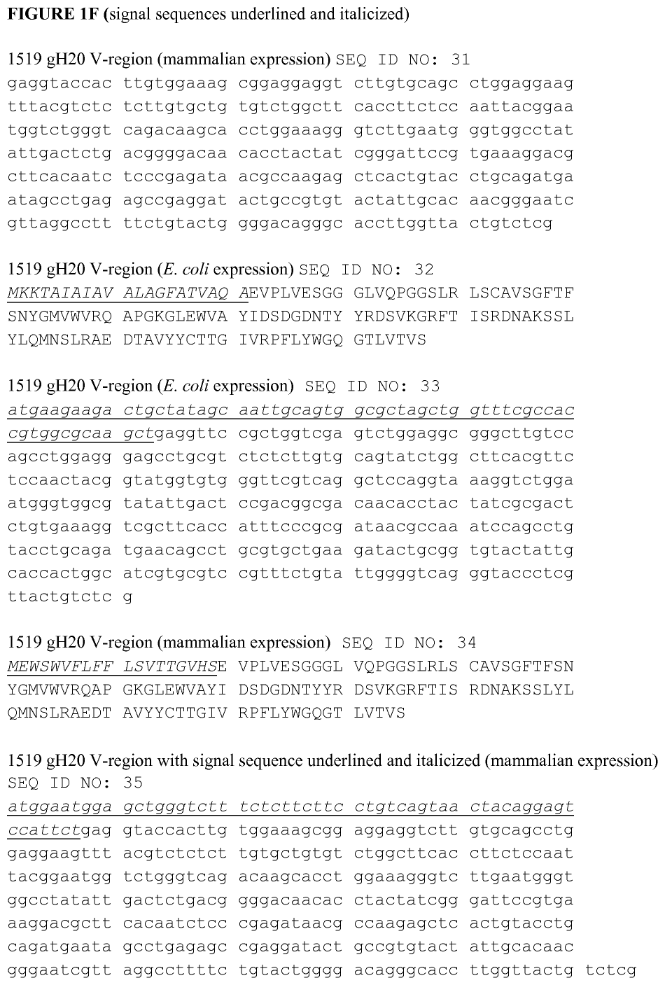

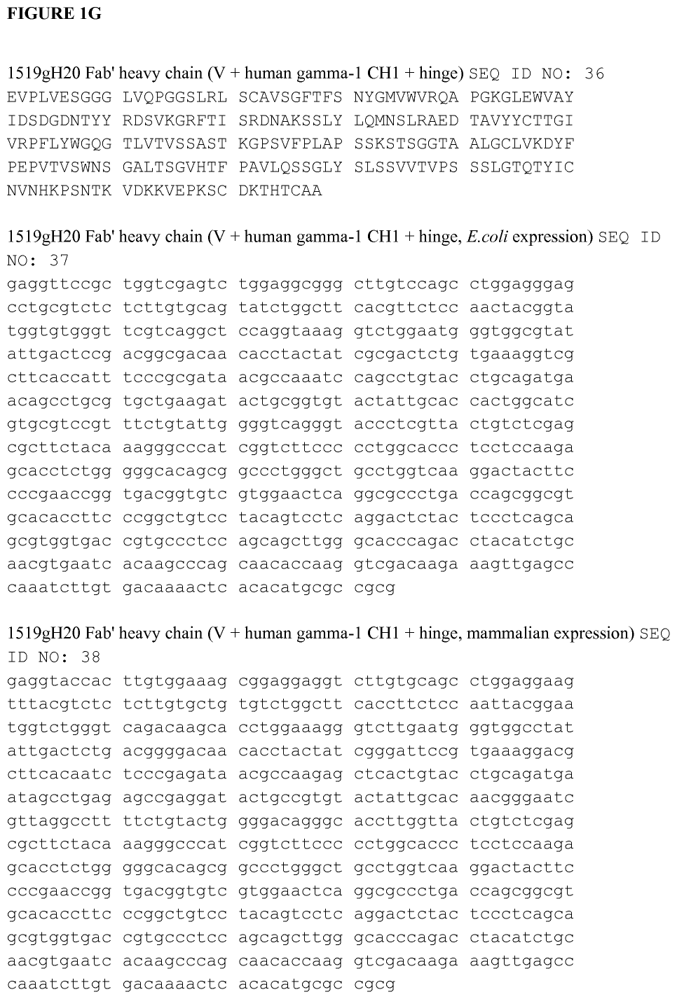

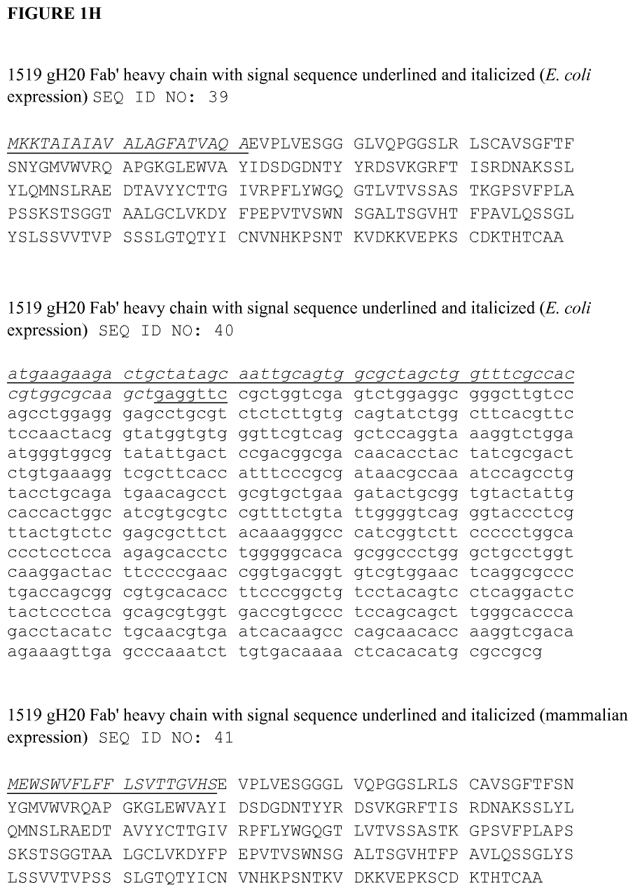

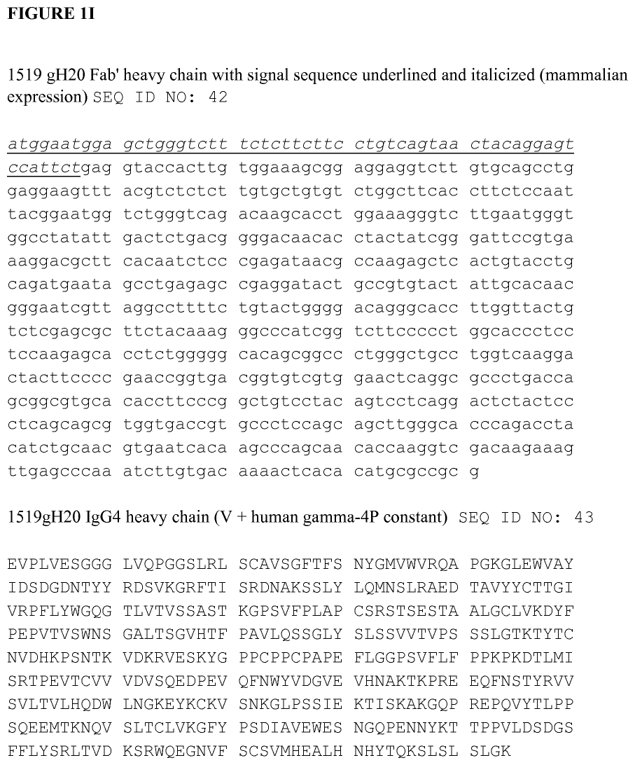

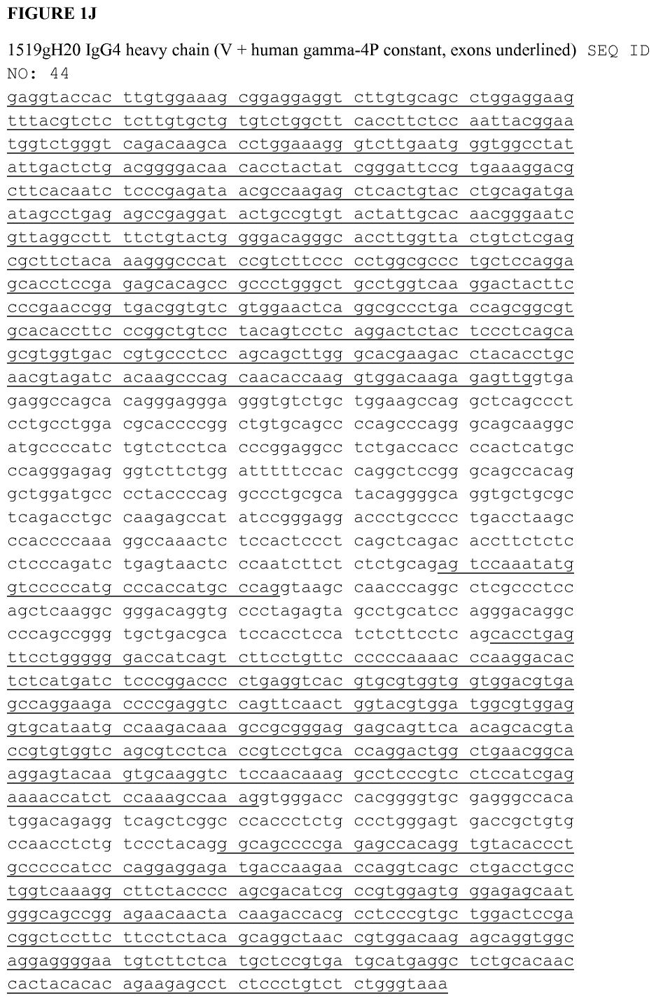

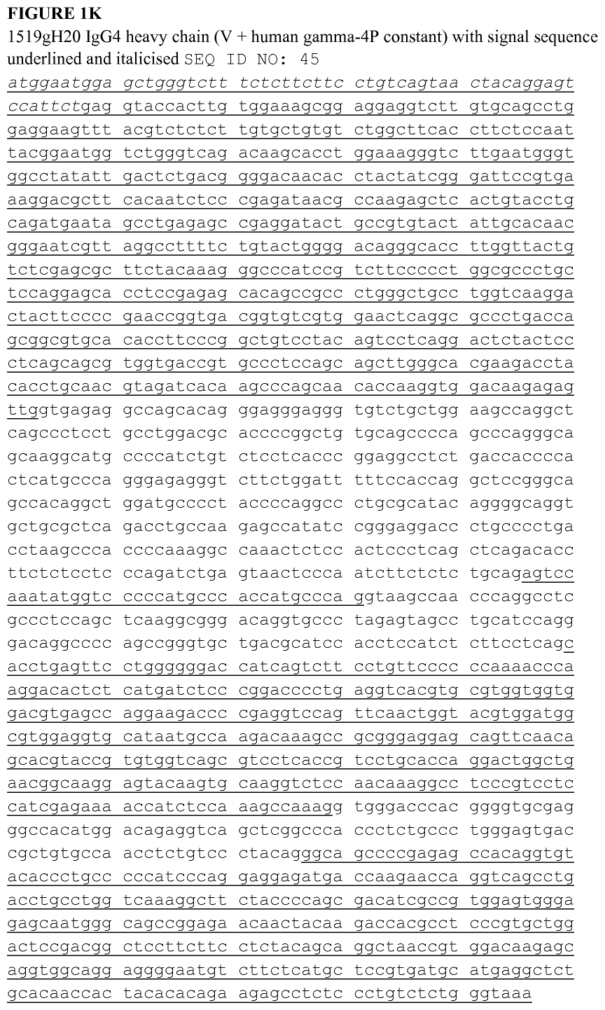

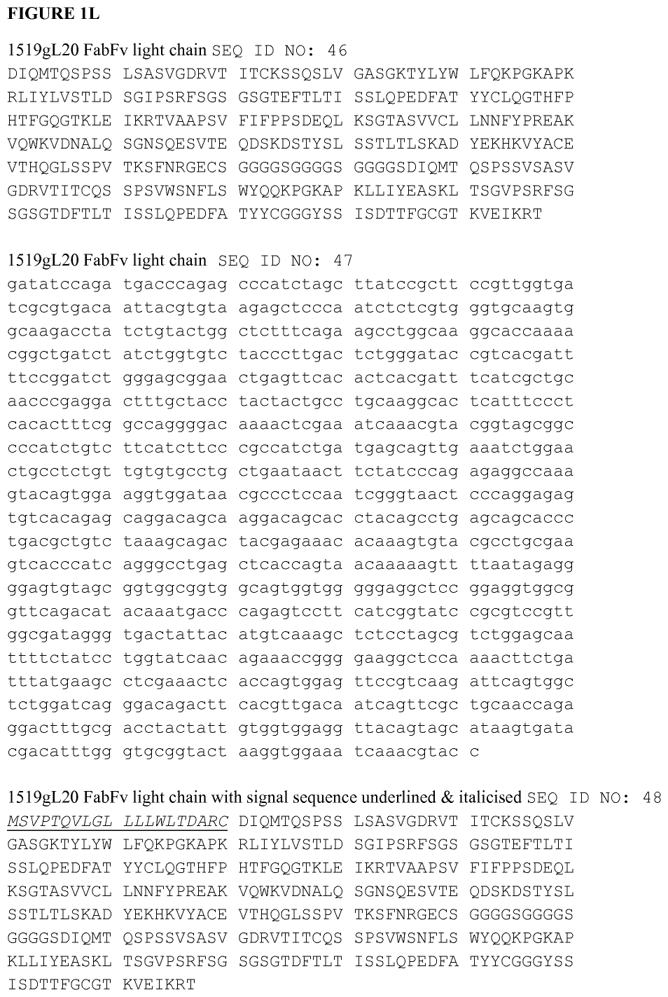

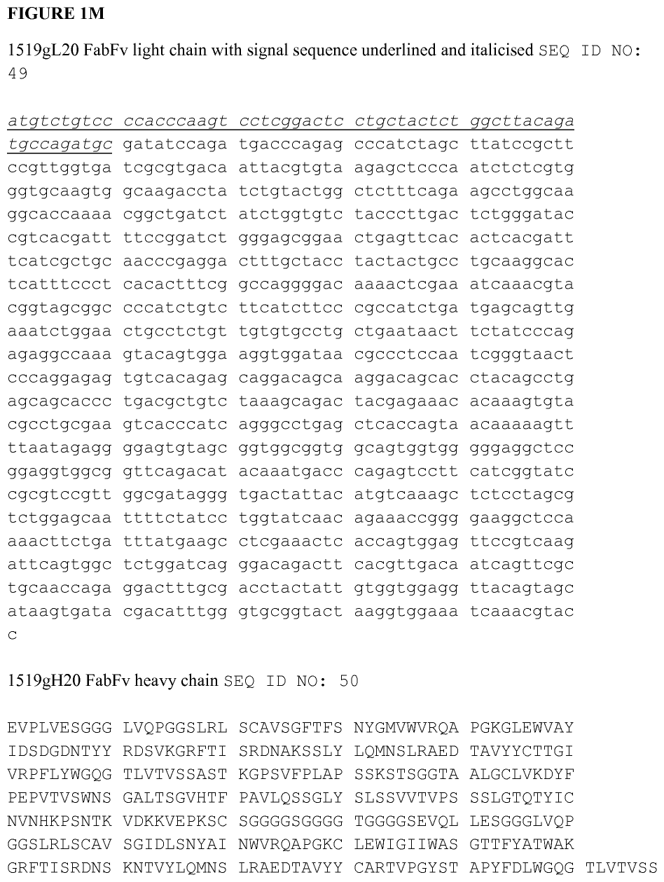

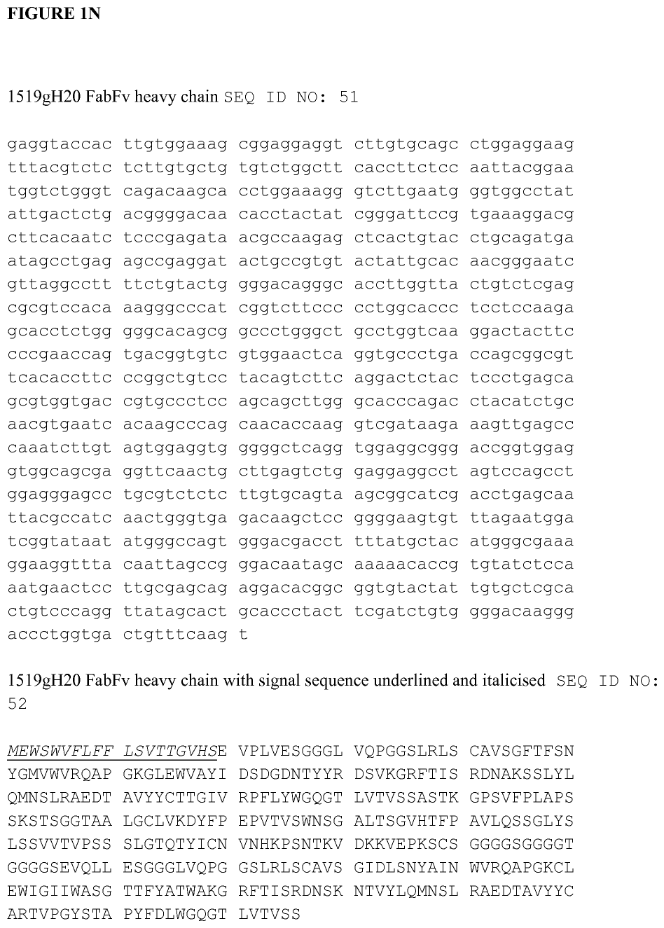

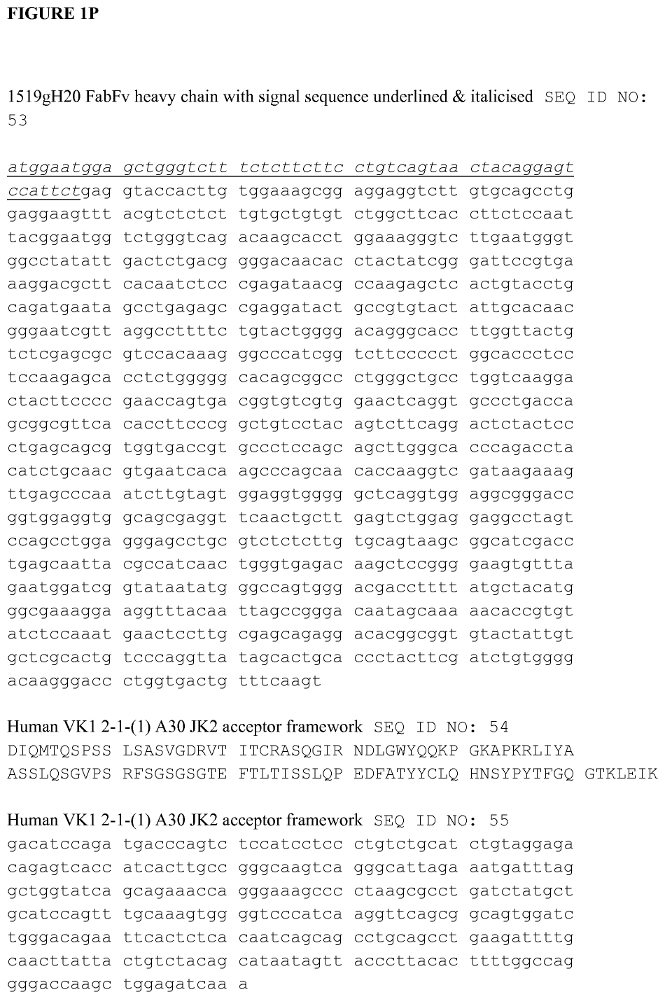

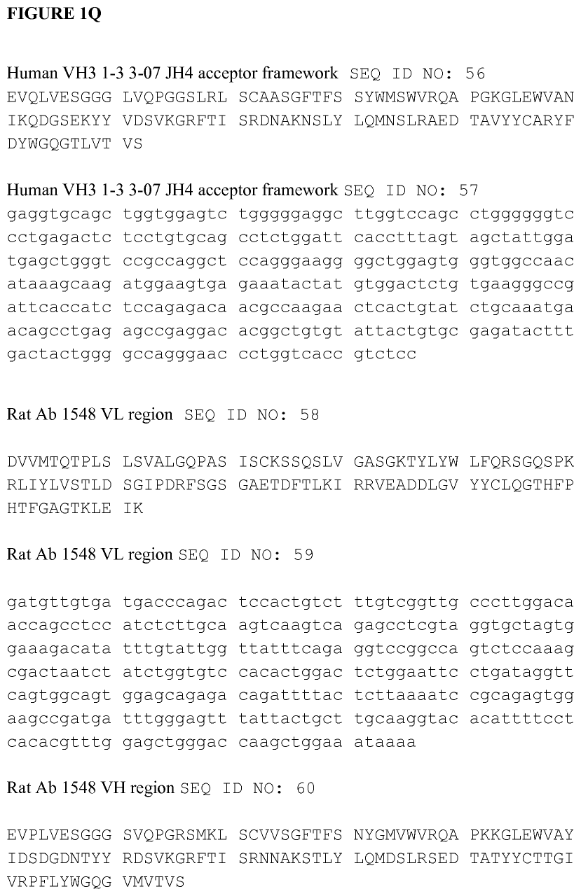

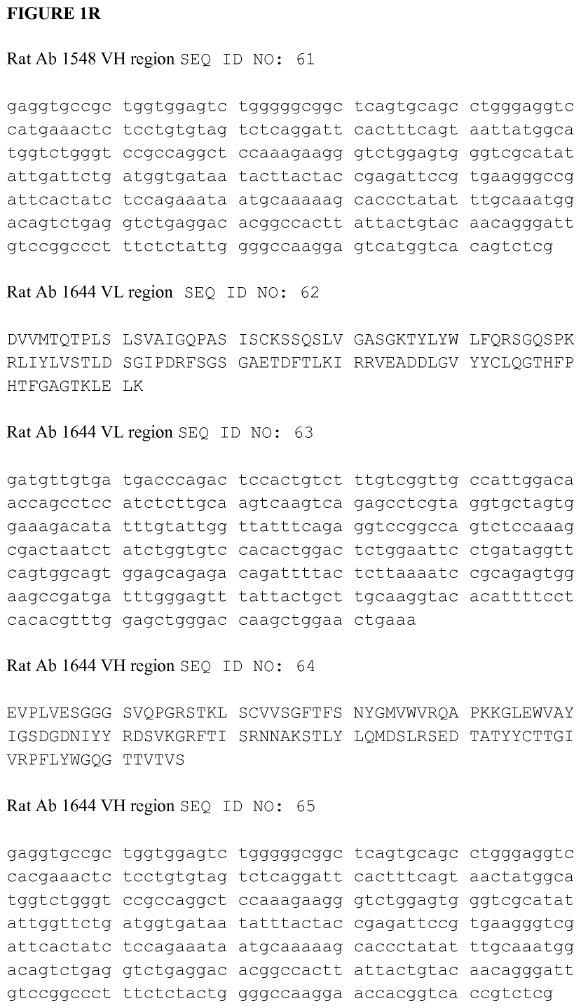

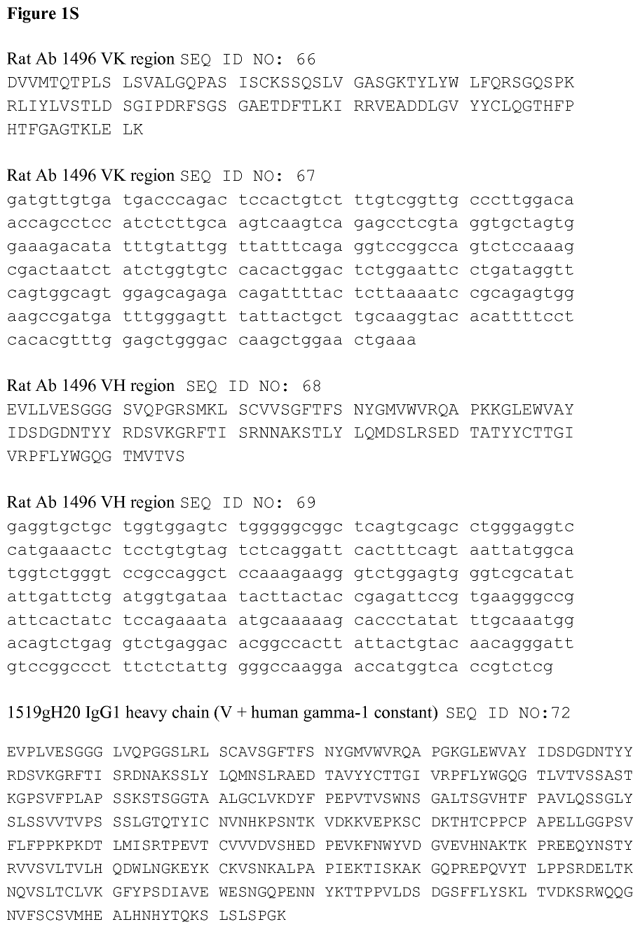

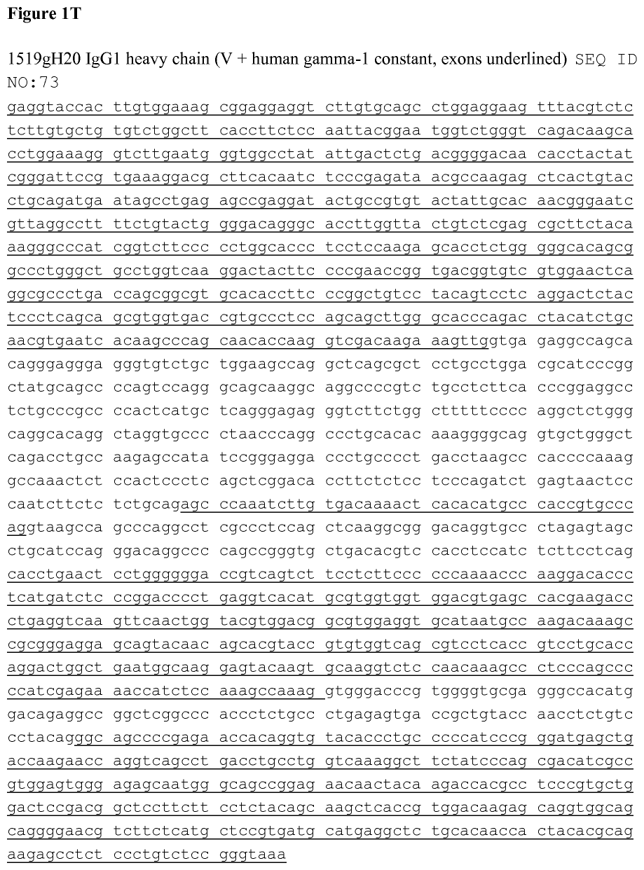

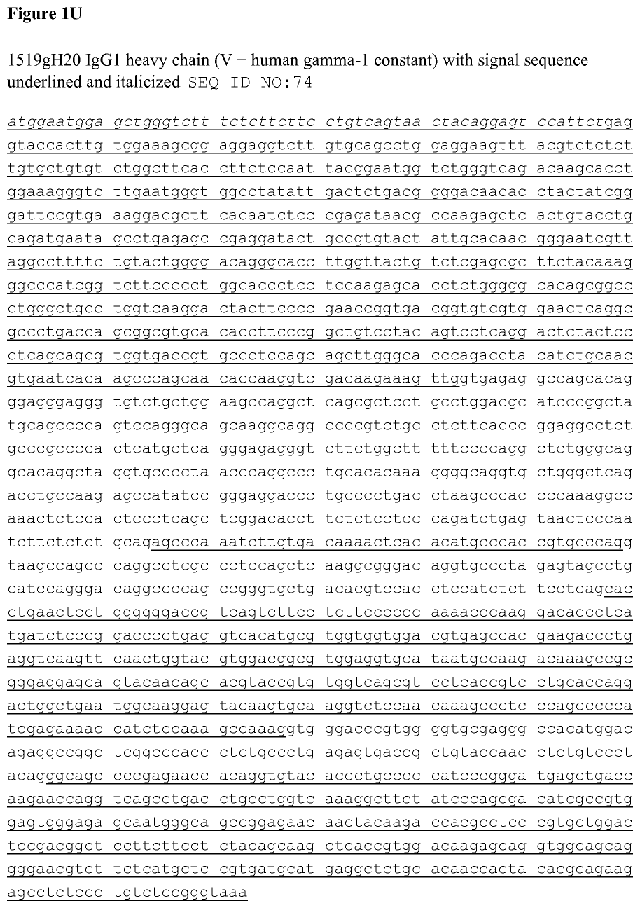

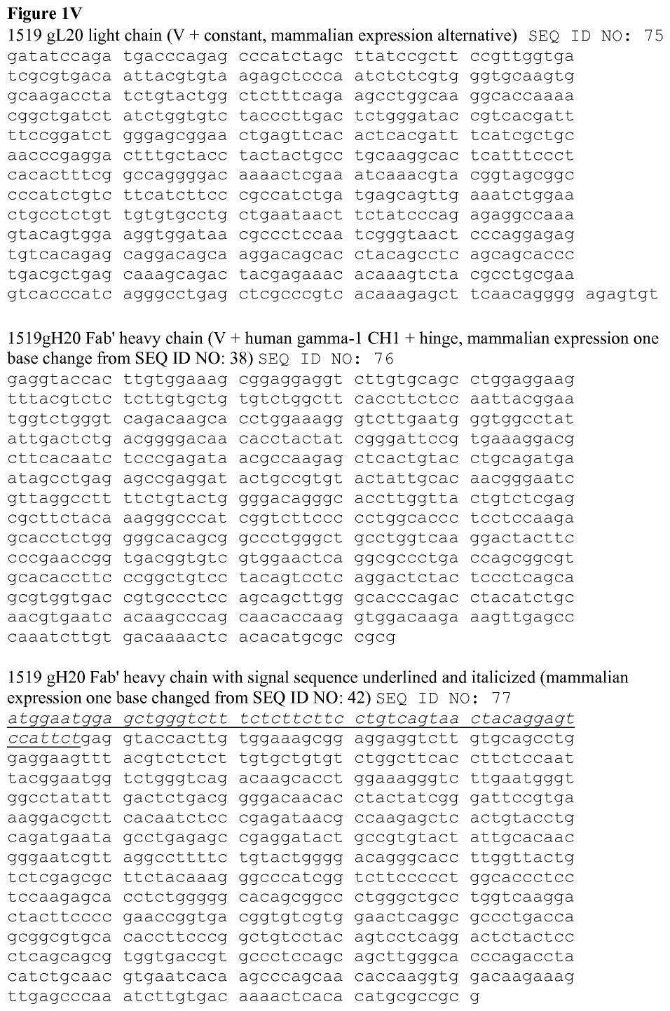

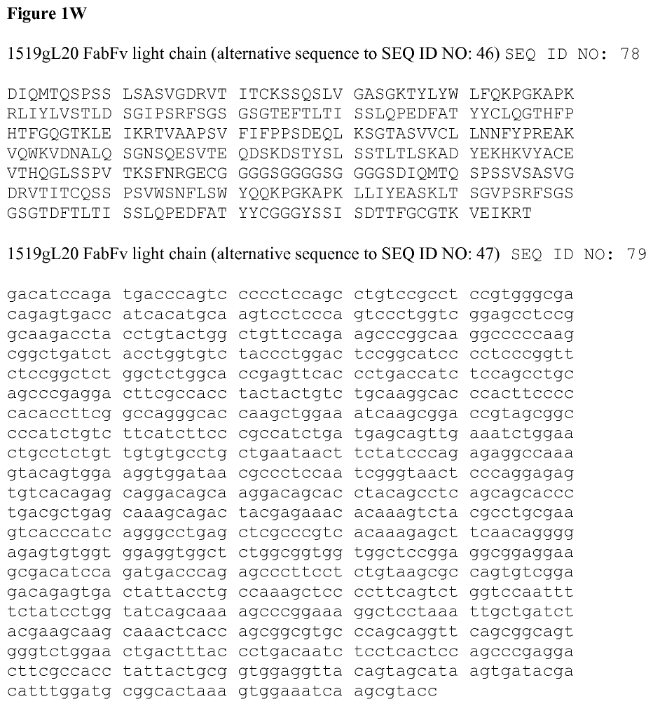

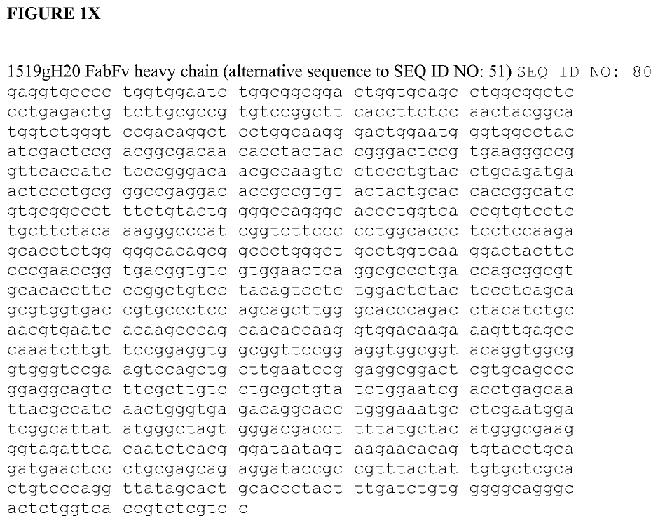

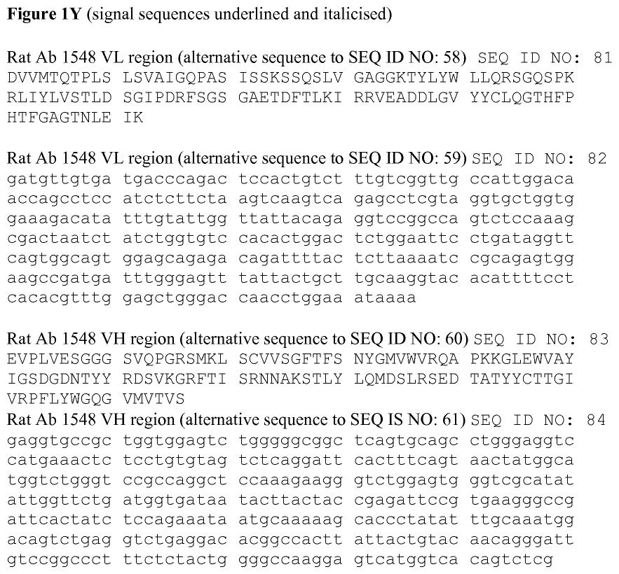

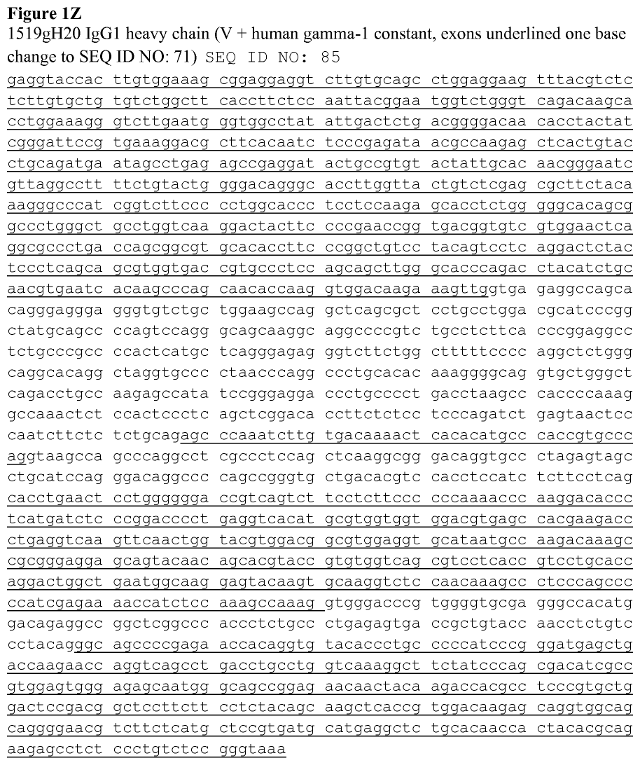

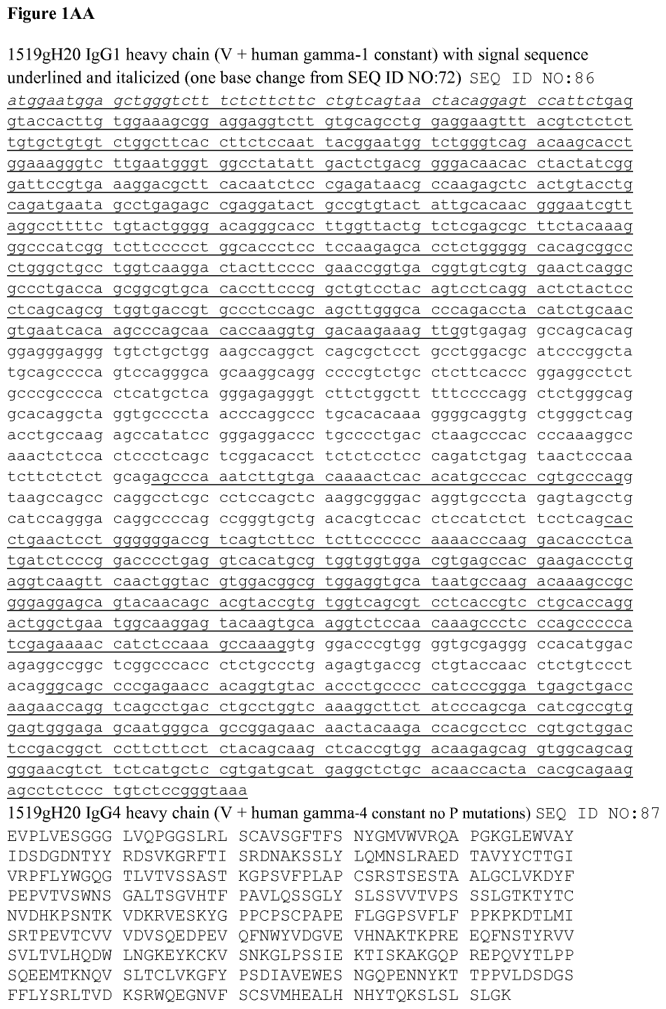

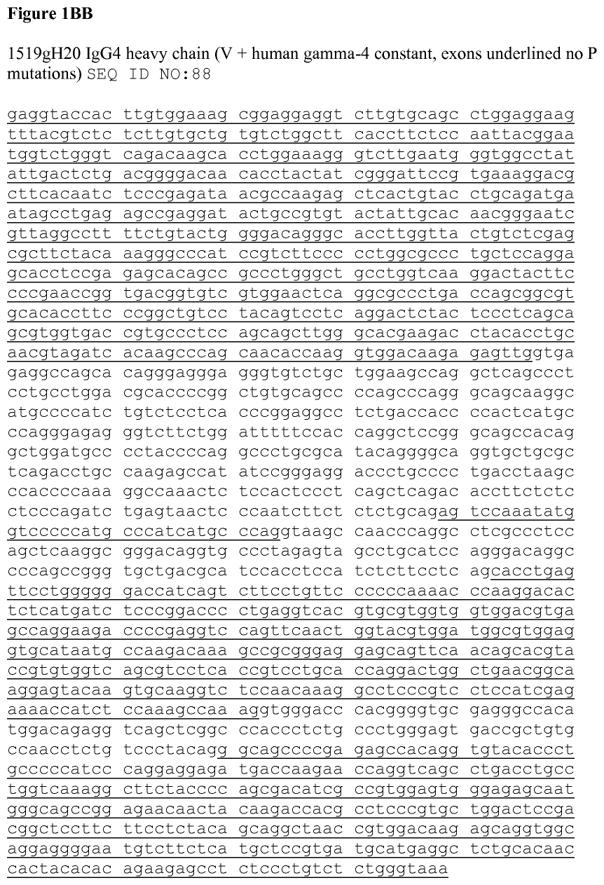

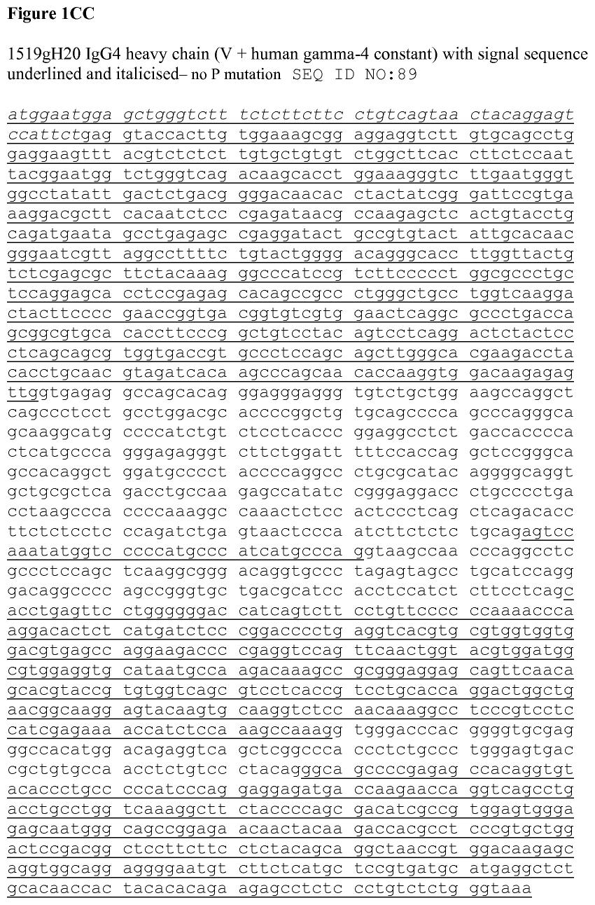

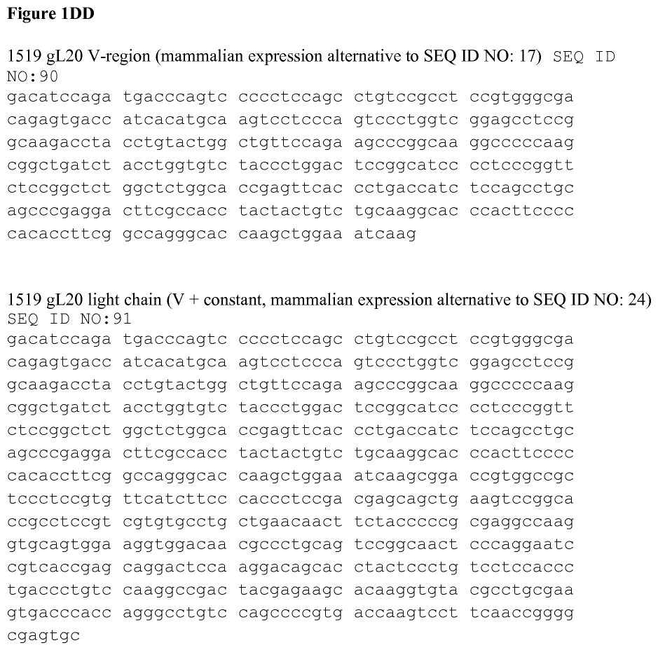

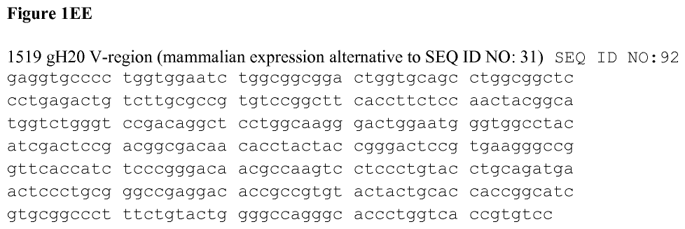

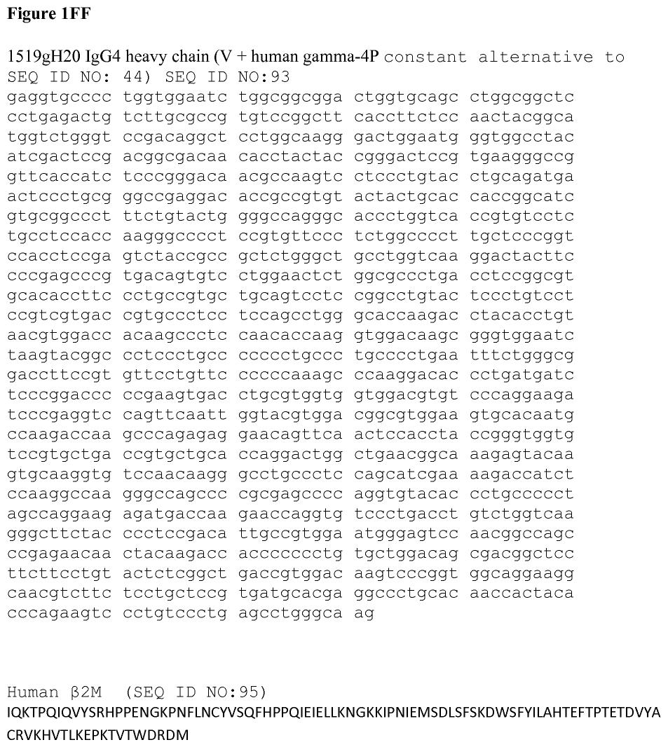

CA170_01519 g57 and 1519 and 1519.g57 are employed inchangeably herein and are used to refer to a specific pair of antibody variable regions which may be used in a number of different formats. These variable regions are the heavy chain sequence given in SEQ ID NO:29 and the light chain sequence given in SEQ ID NO:15 (FIG. 1).

The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used. For example IgG4 molecules in which the serine at position 241 has been changed to proline as described in Angal et al., Molecular Immunology, 1993, 30 (1), 105-108 may be used. It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, R J. Journal of Chromatography 705:129-134, 1995). Accordingly, the C-terminal lysine of the antibody heavy chain may be absent.

In one embodiment the antibody heavy chain comprises a CH1 domain and the antibody light chain comprises a CL domain, either kappa or lambda.

In one embodiment the light chain has the sequence given in SEQ ID NO:22 and the heavy chain has the sequence given in SEQ ID NO:43.

In one embodiment the light chain has the sequence given in SEQ ID NO:22 and the heavy chain has the sequence given in SEQ ID NO:72.

In one embodiment a C-terminal amino acid from the antibody molecule is cleaved during post-translation modifications.

In one embodiment an N-terminal amino acid from the antibody molecule is cleaved during post-translation modifications.

Also provided by the present disclosure is a specific region or epitope of human FcRn which may be bound by an antibody provided by the disclosure, in particular an antibody comprising the heavy chain sequence gH20 (SEQ ID NO:29) and/or the light chain sequence gL20 (SEQ ID NO:15).

This specific region or epitope of the human FcRn polypeptide can be identified by any suitable epitope mapping method known in the art in combination with any one of the antibodies provided by the present invention. Examples of such methods include screening peptides of varying lengths derived from FcRn for binding to the antibody of the present invention with the smallest fragment that can specifically bind to the antibody containing the sequence of the epitope recognised by the antibody. The FcRn peptides may be produced synthetically or by proteolytic digestion of the FcRn polypeptide. Peptides that bind the antibody can be identified by, for example, mass spectrometric analysis. In another example, NMR spectroscopy or X-ray crystallography can be used to identify the epitope bound by an antibody of the present disclosure. Once identified, the epitopic fragment which binds an antibody of the present disclosure can be used, if required, as an immunogen to obtain additional antibodies which bind the same epitope.

In one embodiment an antibody of the present disclosure binds the human FcRn alpha chain extracellular sequence as shown below:

(SEQ ID NO: 94)
AESHLSLLYH LTAVSSPAPG TPAFWVSGWL GPQQYLSYNS
LRGEAEPCGA WVWENQVSWY WEKETTDLRI KEKLFLEAFK
ALGGKGPYTL QGLLGCELGP DNTSVPTAKF
ALNGEEFMNF DLKQGTWGGD WPEALAISQR WQQQDKAANK
ELTFLLFSCP HRLREHLERG RGNLEWKEPP SMRLKARPSS
PGFSVLTCSA FSFYPPELQL RFLRNGLAAG TGQGDFGPNS
DGSFHASSSL TVKSGDEHHY CCIVQHAGLA
QPLRVELESPAKSS.

The residues underlined are those known to be critical for the interaction of human FcRn with the Fc region of human IgG and those residues highlighted in bold are those involved in the interaction of FcRn with the 1519 antibody of the present disclosure comprising the heavy chain sequence gH20 (SEQ ID NO:29) and the light chain sequence gL20 (SEQ ID NO:15).

In one example, an antibody for use in the present invention is an anti-FcRn antibody molecule which binds an epitope of human FcRn which comprises at least one amino acid selected from the group consisting of residues V105, P106, T107, A108 and K109 of SEQ ID NO:94 and at least one residue, for example at least 2, 3, 4, 5, 6, 7, 8, 9 or 10 residues selected from the group consisting of P100, E115, E116, F117, M118, N119, F120, D121, L122, K123, Q124, G128, G129, D130, W131, P132 and E133 of SEQ ID NO:94.

In one example the epitope of the antibody molecule is determined by X-ray crystallography using the FcRn alpha chain extracellular sequence (SEQ ID NO:94) in complex with β2M, as described in the Examples herein.

In one example, an antibody for use in the present invention is an anti-FcRn antibody molecule which binds an epitope of human FcRn which comprises at least one amino acid selected from the group consisting of residues V105, P106, T107, A108 and K109 of SEQ ID NO:94 and at least one residue, for example at least 2, 3, 4, 5, 6, 7, 8, 9 or 10 residues, selected from the group consisting of E115, E116, F117, M118, N119, F120, D121, L122, K123 and Q124 of SEQ ID NO:94.

In one example, an antibody for use in the present invention is an anti-FcRn antibody molecule which binds an epitope of human FcRn which comprises at least two, three, four or five amino acids selected from the group consisting of residues V105, P106, T107, A108 and K109 of SEQ ID NO:94 and at least one residue selected from the group consisting of E115, E116, F117, M118, N119, F120, D121, L122, K123 and Q124 of SEQ ID NO:94.

In one example, an antibody for use in the present invention is an anti-FcRn antibody molecule which binds an epitope of human FcRn which comprises at least one amino acid selected from the group consisting of residues V105, P106, T107, A108 and K109 of SEQ ID NO:94 and at least one residue selected from the group consisting of P100, E115, E116, F117, M118, N119, F120, D121, L122, K123, Q124, G128, G129, D130, W131, P132 and E133 of SEQ ID NO:94.

In one example, an antibody for use in the present invention is an anti-FcRn antibody molecule which binds an epitope of human FcRn which comprises at least one amino acid selected from the group consisting of residues V105, P106, T107, A108 and K109 of SEQ ID NO:94 and at least one residue selected from the group consisting of P100, M118, N119, F120, D121, L122, K123, Q124 and G128 of SEQ ID NO:94.

In one example, an antibody for use in the present invention is an anti-FcRn antibody molecule which binds an epitope of human FcRn which comprises residues V105, P106, T107, A108 and K109 of SEQ ID NO:94 and at least one residue selected from the group consisting of P100, M118, N119, F120, D121, L122, K123, Q124 and G128 of SEQ ID NO:94.

In one example, an antibody for use in the present invention is an anti-FcRn antibody molecule which binds an epitope of human FcRn which comprises residues V105, P106, T107, A108 and K109 of SEQ ID NO:94 and at least one residue selected from the group consisting of P100, E115, E116, F117, M118, N119, F120, D121, L122, K123, Q124, G128, G129, D130, W131, P132 and E133 of SEQ ID NO:94.

In one example, an antibody for use in the present invention is an anti-FcRn antibody molecule which binds an epitope of human FcRn which comprises residues P100, V105, P106, T107, A108 and K109 of SEQ ID NO:94 and at least one residue selected from the group consisting of E115, E116, F117, M118, N119, F120, D121, L122, K123, Q124, G128, G129, D130, W131, P132 and E133 of SEQ ID NO:94.

In one example ‘at least one residue’ may be 1, 2, 3, 4, 5, 6, 7, 8, 9, 10, 11, 12, 13, 14, 15 or 16 residues.

In one example an antibody for use in the present invention is an anti-FcRn antibody molecule which binds an epitope of human FcRn which comprises or consists of residues 100, 105 to 109, 115 to 124 and 129 to 133 of SEQ ID NO: 94.

Antibodies which cross-block the binding of an antibody molecule according to the present disclosure in particular, an antibody molecule comprising the heavy chain sequence given in SEQ ID NO:29 and the light chain sequence given in SEQ ID NO:15 may be similarly useful in blocking FcRn activity. Accordingly, the present disclosure also provides an anti-FcRn antibody molecule for use in the present invention, which cross-blocks the binding of any one of the antibody molecules described herein above to human FcRn and/or is cross-blocked from binding human FcRn by any one of those antibodies. In one embodiment, such an antibody binds to the same epitope as an antibody described herein above. In another embodiment the cross-blocking neutralising antibody binds to an epitope which borders and/or overlaps with the epitope bound by an antibody described herein above.

Cross-blocking antibodies can be identified using any suitable method in the art, for example by using competition ELISA or BIAcore assays where binding of the cross blocking antibody to human FcRn prevents the binding of an antibody of the present invention or vice versa. Such cross blocking assays may use isolated natural or recombinant FcRn or a suitable fusion protein/polypeptide. In one example binding and cross-blocking is measured using recombinant human FcRn extracellular domain (SEQ ID NO:94). In one example the recombinant human FcRn alpha chain extracellular domain is used in a complex with β2 microglobulin (β2M) (SEQ ID NO:95).

In one embodiment there is provided an anti-FcRn antibody molecule for use in the present invention which blocks FcRn binding to IgG and which cross-blocks the binding of an antibody whose heavy chain comprises the sequence given in SEQ ID NO:29 and whose light chain comprises the sequence given in SEQ ID NO:15 to human FcRn. In one embodiment the cross-blocking antibodies provided by the present invention inhibit the binding of an antibody comprising the heavy chain sequence given in SEQ ID NO:29 and the light chain sequence given in SEQ ID NO:15 by greater than 80%, for example by greater than 85%, such as by greater than 90%, in particular by greater than 95%.

Alternatively or in addition, anti-FcRn antibodies according to this aspect of the invention may be cross-blocked from binding to human FcRn by an antibody comprising the heavy chain sequence given in SEQ ID NO:29 and the light chain sequence given in SEQ ID NO:15. Also provided therefore is an anti-FcRn antibody molecule which blocks FcRn binding to IgG and which is cross-blocked from binding human FcRn by an antibody comprising the heavy chain sequence given in SEQ ID NO:29 and the light chain sequence given in SEQ ID NO:15. In one embodiment the anti-FcRn antibodies provided by this aspect of the invention are inhibited from binding human FcRn by an antibody comprising the heavy chain sequence given in SEQ ID NO:29 and the light chain sequence given in SEQ ID NO:15 by greater than 80%, for example by greater than 85%, such as by greater than 90%, in particular by greater than 95%.

In one embodiment the cross-blocking antibodies provided by the present disclosure are fully human. In one embodiment the cross-blocking antibodies provided by the present disclosure are humanised. In one embodiment the cross-blocking antibodies provided by the present disclosure have an affinity for human FcRn of 100 pM or less. In one embodiment the cross-blocking antibodies provided by the present disclosure have an affinity for human FcRn of 50 pM or less. Affinity can be measured using the methods described herein below.

Biological molecules, such as antibodies or fragments, contain acidic and/or basic functional groups, thereby giving the molecule a net positive or negative charge. The amount of overall “observed” charge will depend on the absolute amino acid sequence of the entity, the local environment of the charged groups in the 3D structure and the environmental conditions of the molecule. The isoelectric point (pI) is the pH at which a particular molecule or solvent accessible surface thereof carries no net electrical charge. In one example, the FcRn antibody and fragments of the invention may be engineered to have an appropriate isoelectric point. This may lead to antibodies and/or fragments with more robust properties, in particular suitable solubility and/or stability profiles and/or improved purification characteristics.

Thus in one aspect the disclosure provides a humanised FcRn antibody engineered to have an isoelectric point different to that of the originally identified antibody. The antibody may, for example be engineered by replacing an amino acid residue such as replacing an acidic amino acid residue with one or more basic amino acid residues. Alternatively, basic amino acid residues may be introduced or acidic amino acid residues can be removed. Alternatively, if the molecule has an unacceptably high pI value acidic residues may be introduced to lower the pI, as required. It is important that when manipulating the pI care must be taken to retain the desirable activity of the antibody or fragment. Thus in one embodiment the engineered antibody or fragment has the same or substantially the same activity as the “unmodified” antibody or fragment.

Programs such as ** ExPASY http://www.expasy.ch/tools/pi_tool.html, and http://www.iut-arles.up.univ-mrs.fr/w3bb/d_abim/compo-p.html, may be used to predict the isoelectric point of the antibody or fragment.

The antibody molecules for use in the present invention suitably have a high binding affinity, in particular in the nanomolar range. Affinity may be measured using any suitable method known in the art, including BIAcore, as described in the Examples herein, using isolated natural or recombinant FcRn or a suitable fusion protein/polypeptide. In one example affinity is measured using recombinant human FcRn extracellular domain as described in the Examples herein (SEQ ID NO:94) and in WO2014/019727. In one example affinity is measured using the recombinant human FcRn alpha chain extracellular domain (SEQ ID NO:94) in association with β2 microglobulin (β2M) (SEQ ID NO:95). Suitably the antibody molecules for use in the present invention have a binding affinity for isolated human FcRn of about 1 nM or lower. In one embodiment the antibody molecule of the present invention has a binding affinity of about 500 pM or lower (i.e. higher affinity). In one embodiment the antibody molecule of the present invention has a binding affinity of about 250 pM or lower. In one embodiment the antibody molecule of the present invention has a binding affinity of about 200 pM or lower. In one embodiment the present invention provides an anti-FcRn antibody with a binding affinity of about 100 pM or lower. In one embodiment the present invention provides a humanised anti-FcRn antibody with a binding affinity of about 100 pM or lower. In one embodiment the present invention provides an anti-FcRn antibody with a binding affinity of 50 pM or lower.

Importantly the antibodies for use in the present invention are able to bind human FcRn at both pH6 and pH7.4 with comparable binding affinity. Advantageously therefore the antibodies are able to continue to bind FcRn even within the endosome, thereby maximising the blocking of FcRn binding to IgG.

In one example the present disclosure provides an anti-FcRn antibody with a binding affinity of 100 pM or lower when measured at pH6 and pH7.4. In one example the antibody for use in the invention is an anti-FcRn antibody with a binding affinity of 50 pM or lower when measured at pH6 and pH7.4.

The affinity of an antibody or binding fragment of the present invention, as well as the extent to which a binding agent (such as an antibody) inhibits binding, can be determined by one of ordinary skill in the art using conventional techniques, for example those described by Scatchard et al. (Ann. KY. Acad. Sci. 51:660-672 (1949)) or by surface plasmon resonance (SPR) using systems such as BIAcore. For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al, Cancer Res. 53:2560-65 (1993)).

In the present invention affinity of the test antibody molecule is typically determined using SPR as follows. The test antibody molecule is captured on the solid phase and human FcRn alpha chain extracellular domain in non-covalent complex with β2M is run over the captured antibody in the mobile phase and affinity of the test antibody molecule for human FcRn determined. The test antibody molecule may be captured on the solid phase chip surface using any appropriate method, for example using an anti-Fc or anti Fab′ specific capture agent. In one example the affinity is determined at pH6. In one example the affinity is determined at pH7.4.

It will be appreciated that the affinity of antibodies provided by the present invention may be altered using any suitable method known in the art. The present invention therefore also relates to variants of the antibody molecules of the present invention, which have an improved affinity for FcRn. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998). Vaughan et al. (supra) discusses these methods of affinity maturation.

In one embodiment the antibody molecules for use in the present invention block human FcRn activity. Assays suitable for determining the ability of an antibody to block FcRn are described in WO2014/019727. Suitable assays for determining whether antibodies block FcRn interaction with circulating IgG molecules are also described in WO2014/019727 along with a suitable assay for determining the ability of an antibody molecule to block IgG recycling in vitro.

If desired an antibody for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector molecule is a protein or polypeptide the linkage may be achieved using recombinant DNA procedures, for example as described in WO 86/01533 and EP0392745.

The term effector molecule as used herein includes, for example, antineoplastic agents, drugs, toxins, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Examples of effector molecules may include cytotoxins or cytotoxic agents including any agent that is detrimental to (e.g. kills) cells. Examples include combrestatins, dolastatins, epothilones, staurosporin, maytansinoids, spongistatins, rhizoxin, halichondrins, roridins, hemiasterlins, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Effector molecules also include, but are not limited to, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine).

Other effector molecules may include chelated radionuclides such as ¹¹¹In and ⁹⁰Y, Lu¹⁷⁷, Bismuth²¹³, Californium²⁵², Iridium¹⁹² and Tungsten¹⁸⁸/Rhenium¹⁸⁸; or drugs such as but not limited to, alkylphosphocholines, topoisomerase I inhibitors, taxoids and suramin.

Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

In one embodiment the polymer is albumin or a fragment thereof, such as human serum albumin or a fragment thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired, but will generally be in an average molecular weight range from 500 Da to 50000 Da, for example from 5000 to 40000 Da such as from 20000 to 40000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545). Thus, for example, where the product is intended to leave the circulation and penetrate tissue, for example for use in the treatment of a tumour, it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule.

Suitably, the additional amino acids form a modified hinge region containing one or more cysteine residues to which the effector molecule may be attached. Multiple sites can be used to attach two or more PEG molecules.

Suitably PEG molecules are covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond. Where a thiol group is used as the point of attachment appropriately activated effector molecules, for example thiol selective derivatives such as maleimides and cysteine derivatives may be used. An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, Ala., USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, the antibody is a modified Fab fragment, Fab′ fragment or diFab which is PEGylated, i.e. has PEG (poly(ethyleneglycol)) covalently attached thereto, e.g. according to the method disclosed in EP 0948544 or EP1090037 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington D.C. and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one example PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

Particular PEG molecules include 2-[3-(N-maleimido)propionamido]ethyl amide of N,N′-bis(methoxypoly(ethylene glycol) MW 20,000) modified lysine, also known as PEG2MAL40K (obtainable from Nektar, formerly Shearwater).

Alternative sources of PEG linkers include NOF who supply GL2-400MA3 (wherein m in the structure below is 5) and GL2-400MA (where m is 2) and n is approximately 450:

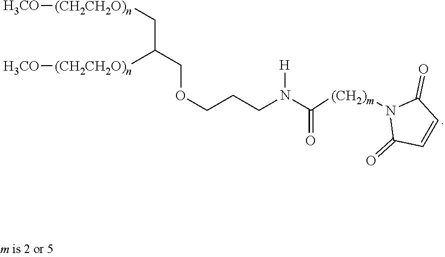

That is to say each PEG is about 20,000 Da.

Thus in one embodiment the PEG is 2,3-Bis(methylpolyoxyethylene-oxy)-1-{[3-(6-maleimido-1-oxohexyl)amino]propyloxy}hexane (the 2 arm branched PEG, —CH₂)₃NHCO(CH₂)₅-MAL, Mw 40,000 known as SUNBRIGHT GL2-400MA3.

Further alternative PEG effector molecules of the following type:

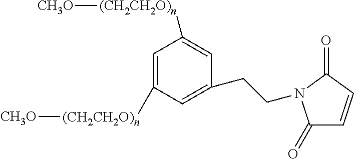

are available from Dr Reddy, NOF and Jenkem.

In one embodiment there is provided an antibody which is PEGylated (for example with a PEG described herein), attached through a cysteine amino acid residue at or about amino acid 226 in the chain, for example amino acid 226 of the heavy chain (by sequential numbering), for example amino acid 226 of SEQ ID NO:36.

In one embodiment the present disclosure provides a Fab′PEG molecule comprising one or more PEG polymers, for example 1 or 2 polymers such as a 40 kDa polymer or polymers.

In one embodiment there is provided a Fab′ conjugated to a polymer, such as a PEG molecule, a starch molecule or an albumin molecule.

In one embodiment the antibody or fragment is conjugated to a starch molecule, for example to increase the half life. Methods of conjugating starch to a protein as described in U.S. Pat. No. 8,017,739 incorporated herein by reference.

The present disclosure also provides an isolated DNA sequence encoding the heavy and/or light chain(s) of an antibody molecule of the present invention. Suitably, the DNA sequence encodes the heavy or the light chain of an antibody molecule of the present invention. The DNA sequence of the present invention may comprise synthetic DNA, for instance produced by chemical processing, cDNA, genomic DNA or any combination thereof.

DNA sequences which encode an antibody molecule of the present invention can be obtained by methods well known to those skilled in the art. For example, DNA sequences coding for part or all of the antibody heavy and light chains may be synthesised as desired from the determined DNA sequences or on the basis of the corresponding amino acid sequences.

DNA coding for acceptor framework sequences is widely available to those skilled in the art and can be readily synthesised on the basis of their known amino acid sequences.

Standard techniques of molecular biology may be used to prepare DNA sequences coding for the antibody molecule of the present invention. Desired DNA sequences may be synthesised completely or in part using oligonucleotide synthesis techniques. Site-directed mutagenesis and polymerase chain reaction (PCR) techniques may be used as appropriate.

Examples of suitable DNA sequences are provided in herein.

Examples of suitable DNA sequences encoding the 1519 light chain variable region are provided in SEQ ID NO:16, SEQ ID NO:17 and SEQ ID NO:90. Examples of suitable DNA sequences encoding the 1519 heavy chain variable region are provided in SEQ ID NO:30, SEQ ID NO:31 and SEQ ID NO:92.

Examples of suitable DNA sequences encoding the 1519 light chain (variable and constant) are provided in SEQ ID NO:23, SEQ ID NO:75 and SEQ ID NO:91.

Examples of suitable DNA sequences encoding the 1519 heavy chain (variable and constant, depending on format) are provided in SEQ ID NOs:37, 38 and 76 (Fab′), SEQ ID NO:72 or 85 (IgG1), SEQ ID NO: 44 or 93 (IgG4P) and SEQ ID:88 (IgG4).

The present disclosure also relates to a cloning or expression vector comprising one or more DNA sequences of the present invention. Accordingly, provided is a cloning or expression vector comprising one or more DNA sequences encoding an antibody of the present invention. Suitably, the cloning or expression vector comprises two DNA sequences, encoding the light chain and the heavy chain of the antibody molecule of the present invention, respectively and suitable signal sequences. In one example the vector comprises an intergenic sequence between the heavy and the light chains (see WO03/048208).

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

Also provided is a host cell comprising one or more cloning or expression vectors comprising one or more DNA sequences encoding an antibody of the present invention. Any suitable host cell/vector system may be used for expression of the DNA sequences encoding the antibody molecule of the present invention. Bacterial, for example E. coli, and other microbial systems may be used or eukaryotic, for example mammalian, host cell expression systems may also be used. Suitable mammalian host cells include CHO, myeloma or hybridoma cells.

Suitable types of Chinese Hamster Ovary (CHO cells) for use in the present invention may include CHO and CHO-K1 cells including dhfr− CHO cells, such as CHO-DG44 cells and CHO-DXB11 cells and which may be used with a DHFR selectable marker or CHOK1-SV cells which may be used with a glutamine synthetase selectable marker. Other cell types of use in expressing antibodies include lymphocytic cell lines, e.g., NSO myeloma cells and SP2 cells, COS cells.

The present disclosure also provides a process for the production of an antibody molecule according to the present invention comprising culturing a host cell containing a vector of the present invention under conditions suitable for leading to expression of protein from DNA encoding the antibody molecule of the present invention, and isolating the antibody molecule.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Antibodies for use in the present invention may be provided as a pharmaceutical or diagnostic composition comprising an antibody molecule of the present disclosure in combination with one or more of a pharmaceutically acceptable excipient, diluent or carrier. The composition will usually be supplied as part of a sterile, pharmaceutical composition that will normally include a pharmaceutically acceptable carrier. A pharmaceutical composition of the present invention may additionally comprise a pharmaceutically-acceptable excipient.

The present disclosure also provides a process for preparation of a pharmaceutical or diagnostic composition comprising adding and mixing the antibody molecule of the present invention together with one or more of a pharmaceutically acceptable excipient, diluent or carrier.

The antibody molecule may be the sole active ingredient in the pharmaceutical or diagnostic composition or may be accompanied by other active ingredients including other antibody ingredients or non-antibody ingredients such as steroids or other drug molecules, in one example these are drug molecules whose half-life is independent of FcRn binding.

Pharmaceutical compositions may be conveniently presented in unit dose forms containing a predetermined amount of an active agent of the invention per dose.

Therapeutic doses of the antibodies according to the present disclosure show no apparent toxicology effects in vivo.

Compositions may be administered individually to a patient or may be administered in combination (e.g. simultaneously, sequentially or separately) with other agents, drugs or hormones. In one embodiment the antibodies or antigen binding fragments according to the present disclosure are employed with a cholinesterase inhibitor, an immunosuppressive or an immunomodulatory agent.

In one embodiment the antibodies or antigen binding fragments according to the present disclosure are employed with an immunosuppressant therapy, such as a steroid, in particular prednisone. In one embodiment the antibodies or antigen binding fragments according to the present disclosure are employed with a cholinesterase inhibitor, such as pyridostigmine.

In one embodiment the antibodies or antigen binding fragments according to the present disclosure are employed with immunosuppressants such as Cyclosporin or Tacrolimus.

In one embodiment the antibodies or fragments according to the present disclosure are employed with Rituximab or other B cell therapies.

In one embodiment the antibodies or fragments according to the present disclosure are employed with any B cell or T cell modulating agent or immunomodulator. Examples include methotrexate, microphenyolate and azathioprine.

Examples of suitable concomitant therapies are described in the Examples herein. In one example a biologic is not permitted as a concomitant therapy.

The pharmaceutically acceptable carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Suitable forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the antibody molecule may be in dry form, for reconstitution before use with an appropriate sterile liquid.

Once formulated, the compositions of the invention can be administered directly to the subject. The subjects to be treated can be animals. However, in one or more embodiments the compositions are adapted for administration to human subjects.

Suitably in formulations according to the present disclosure, the pH of the final formulation is not similar to the value of the isoelectric point of the antibody or fragment, for example if the pI of the protein is in the range 8-9 or above then a formulation pH of 7 may be appropriate. Whilst not wishing to be bound by theory it is thought that this may ultimately provide a final formulation with improved stability, for example the antibody or fragment remains in solution.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. Dosage treatment may be a single dose schedule or a multiple dose schedule. Preferably the delivery is subcutaneous. In one example the delivery is by subcutaneous infusion. In one example the delivery is not intravenous.

It will be appreciated that the active ingredient in the composition will be an antibody molecule. As such, it will be susceptible to degradation in the gastrointestinal tract. Thus, if the composition is to be administered by a route using the gastrointestinal tract, the composition will need to contain agents which protect the antibody from degradation but which release the antibody once it has been absorbed from the gastrointestinal tract.

A thorough discussion of pharmaceutically acceptable carriers is available in Remington's Pharmaceutical Sciences (Mack Publishing Company, N.J. 1991).

The antibody of the invention can be delivered dispersed in a solvent, e.g., in the form of a solution or a suspension. It can be suspended in an appropriate physiological solution, e.g., saline or other pharmacologically acceptable solvent or a buffered solution. A suspension can employ, for example, lyophilised antibody.

The therapeutic suspensions or solution formulations can also contain one or more excipients. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. The formulation will generally be provided in a substantially sterile form employing sterile manufacture processes.

This may include production and sterilization by filtration of the buffered solvent/solution used for the formulation, aseptic suspension of the antibody in the sterile buffered solvent solution, and dispensing of the formulation into sterile receptacles by methods familiar to those of ordinary skill in the art.

Once formulated, the compositions of the disclosure can be administered directly to the human subject, although the disclosure also contemplates that the method may be employed on non-human subjects. In the present disclosure, the human suffers from Myasthenia Gravis (MG), in one example generalised Myasthenia Gravis, in one example moderate to severe MG, in one example moderate to severe generalised MG. The clinical classification tool for MG is set out below, known as the MG Foundation of America (MGFA) Clinical Classification (Jaretzki et al, 2000). This is a 5-stage classification (I to V), with a higher class indicating more severe disease. Typically moderate to severe is classified as class II-class IVa.

### MGFA Clinical Classification

Class I: Any ocular muscle weakness; may have weakness of eye closure. All other muscle strength is normal.

Class II: Mild weakness affecting muscles other than ocular muscles; may also have ocular muscle weakness of any severity.
A. IIa. Predominantly affecting limb, axial muscles, or both. May also have lesser involvement of oropharyngeal muscles.
B. IIb. Predominantly affecting oropharyngeal, respiratory muscles, or both. May also have lesser or equal involvement of limb, axial muscles, or both.

Class III: Moderate weakness affecting muscles other than ocular muscles; may also have ocular muscle weakness of any severity.
A. IIIa. Predominantly affecting limb, axial muscles, or both. May also have lesser involvement of oropharyngeal muscles.
B. IIIb. Predominantly affecting oropharyngeal, respiratory muscles, or both. May also have lesser or equal involvement of limb, axial muscles, or both.

Class IV: Severe weakness affecting muscles other than ocular muscles; may also have ocular muscle weakness of any severity.
A. IVa. Predominantly affecting limb, axial muscles, or both. May also have lesser involvement of oropharyngeal muscles.
B. IVb. Predominantly affecting oropharyngeal, respiratory muscles, or both. May also have lesser or equal involvement of limb, axial muscles, or both.

Class V: Defined as intubation, with or without mechanical ventilation, except when employed during routine postoperative management. The use of a feeding tube without intubation places the patient in class IVb.

The major pathophysiology leading to MG is the abnormal production of IgG autoantibodies directed toward nicotinic acetylcholine receptor (AChR) or muscle-specific kinase (MuSK) protein and both can be measured using standard methods known in the art, such as radioimmunoprecipitation, ELISA and cell-based assays.

Accordingly, in one example the human is anti-AChR and/or anti-MuSK autoantibody-positive. In one example of the present disclosure the human has moderate to severe generalised MG and is anti-AChR and/or anti-MuSK autoantibody-positive. In one example the human has moderate to severe generalised MG, is anti-AChR and/or anti-MuSK autoantibody-positive and is being considered for treatment with IVIg or plasma exchange (PLEX).

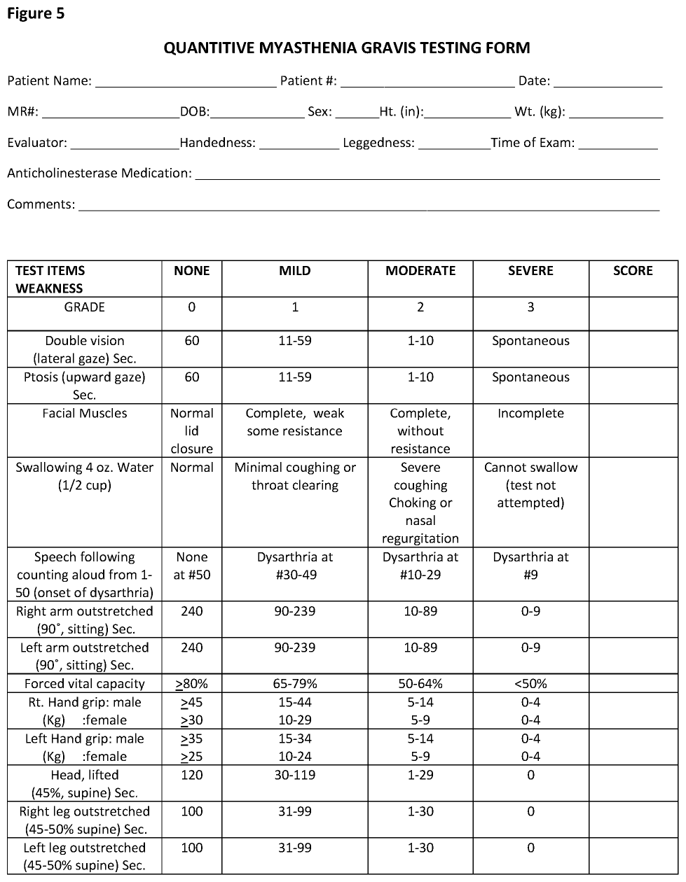

Formal recommendations for clinical research standards identified a need for validated, disease-specific measures to assess therapeutic responses in MG clinical trials, including patient-reported functional and quality-of-life quality of life measures. These recommendations led to validation studies of the Quantitative MG score (QMG), MG-Activities of Daily Living Profile (MG-ADL) and the MG Composite score (MG-C). These measures provided herein and in FIGS. 5, 6 and 7, provide a consistent methodology for assessing clinical response and include patient-centered outcomes. These outcome measures, particularly the QMG and MG-ADL, are being used as primary endpoints in clinical trials for new MG therapies

Accordingly, in one example a human has a diagnosis of moderate to severe generalized myasthenia gravis, is anti-AChR and/or anti-MuSK autoantibody-positive and/or has a myasthenia gravis-activities of daily living (MG-ADL) score of at least 3 and/or a quantitative myasthenia gravis (QMG) score of at least 11.

In one example a human has a diagnosis of generalized myasthenia gravis, is anti-AChR and/or anti-MuSK autoantibody-positive, has a Myasthenia Gravis Foundation of America (MGFA) Class II to IVa and/or has a myasthenia gravis-activities of daily living (MG-ADL) score of at least 3 and/or a quantitative myasthenia gravis (QMG) score of at least 11.

Treatment of MG remains a difficult clinical problem, requiring the long-term use of high-dose corticosteroids alone or combined with cytotoxic agents. Many of the therapies thought to be effective in MG have insufficient data to clearly support their use, are not effective in all patients and conditions, and have broad immunosuppressive effects causing considerable toxicity and treatment-related morbidity. Moreover, due to the natural fluctuations in the course of the disease, many patients need an effective treatment for acute situations requiring urgent treatment.

Both PLEX and IVIg currently are used as the standard of care to improve symptoms in situations requiring chronic-intermittent treatment; however, neither treatment is approved in the US for MG, and the procedures often are burdensome for the patients. Thus a significant unmet medical need exists in this patient population for an effective chronic-intermittent treatment with increased convenience for patients with generalized MG.

In one example, such patients are no longer responding to other therapies, such as immunosuppressants.

Goals for treatment in MG are therefore to provide a chronic intermittent treatment (for disease flares) and/or a treatment for long term maintenance.

Accordingly, in one example, the method of treatment of the present invention may be used for the chronic-intermittent treatment of MG.

Accordingly, in one example, the method of treatment of the present invention may be used for the long term maintenance treatment of MG.

In one example, the method of treatment of the present invention may be used for both chronic-intermittent and long-term maintenance treatment of MG.

### Administration Regimens

The composition preferably comprises a therapeutically effective amount of the antibody (or antigen binding fragment thereof). The term “therapeutically effective amount” as used herein refers to an amount of a therapeutic agent needed to treat, ameliorate or prevent a targeted disease or condition, or to exhibit a detectable therapeutic or preventative effect. “Administration regimen” contemplates the amount (dose) of antibody or fragment thereof administered as well as timing of administration if multiple doses are provided.

Several methods of characterizing MG and treatment efficacy exist and are appropriate for detecting a positive biological response in the subject, and these are further described in the Examples herein.

One assessment is the Quantitative Myasthenia Gravis score (QMG) score (FIG. 5). The QMG is a validated assessment (Barnett et al, 2012), with a higher score indicating more severe disease. Scoring for each item ranges from no weakness (0) to severe weakness (3), with an overall score range from 0 to 39. A 3 point change in the total score is considered clinically relevant.

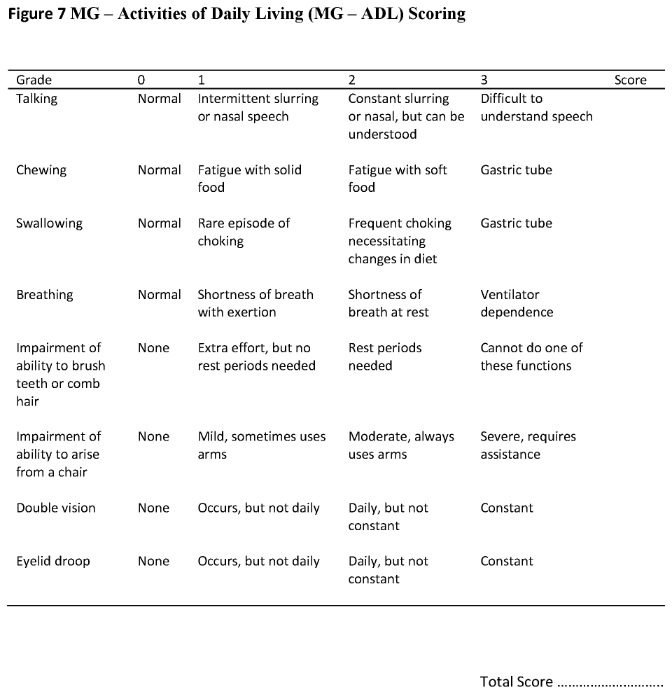

One assessment is the Myasthenia Gravis Activities of Daily Living (MG-ADL) score (FIG. 7). The MGADL is an 8-item PRO instrument developed on the basis of the QMG (Wolfe et al, 1999). The MGADL targets symptoms and disability across ocular, bulbar, respiratory, and axial symptoms. In a recent study, reliability, validity, and responsiveness of the MGADL were further assessed and it was demonstrated that a 2-point improvement indicates clinical improvement (Muppidi, 2012; Muppidi et al, 2011). The total MGADL score ranges from 0 to 24, with a higher score indicating more disability.

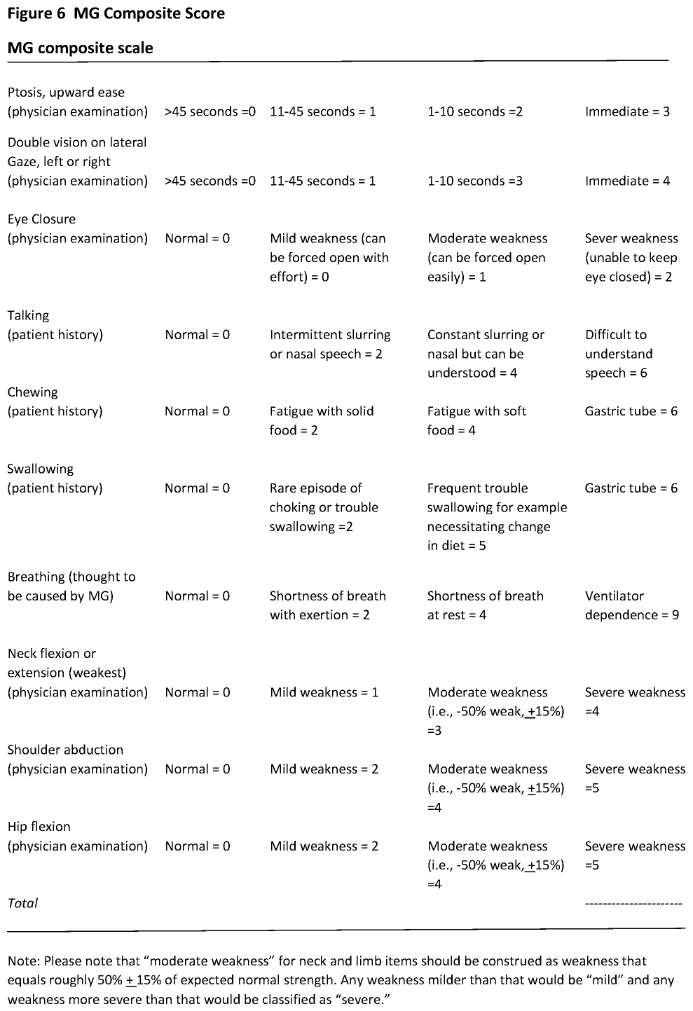

One assessment is the MG Composite score (FIG. 6). The MG-Composite scale is a validated assessment (Burns et al, 2010 MG Composite and MG-QOL15 Study Group. The MGComposite: A valid and reliable outcome measure for myasthenia gravis. Neurology. 2010 May 4; 74(18):1434-40), with a higher score indicating more severe disease and a 3-point change being of clinical relevance. The scale tests 10 items, with individual items being weighted differently. The overall score ranges from 0 to 50.

Efficacy of treatment for MG can be determined by a reduction (compared to baseline) in the Myasthenia Gravis Activities of Daily Living (MG-ADL) score and/or a reduction in the Quantitative Myasthenia Gravis score (QMG) and/or a reduction in the MG composite score, as described in the examples herein.

Treatment efficacy (clinical response) is determined by measuring a change from Baseline. For example, a QMG responder demonstrates a ≥3.0 point improvement from Baseline.

In one example, an MG-Composite responder demonstrates a ≥3.0 point improvement from Baseline.

In one example, an MG-Composite responder demonstrates a ≥5.0 point improvement from Baseline.

In one example, an MG-ADL responder demonstrates a ≥3.0 point improvement from Baseline.

In one example, an MG-ADL responder demonstrates a ≥2.0 point improvement from Baseline.

Other assessments include measuring a reduction in IgG serum levels and/or a reduction in MG-specific autoantibody (anti-MuSK and/or anti-AChR antibody) levels in serum.

As used herein, “treating” and “treatment” refers to any reduction in the severity of MG and “preventing” or “prevention” refers to any reduction or delay in the onset of symptoms of MG. One of ordinary skill in the art will appreciate that any degree of protection from, or amelioration of, MG or symptom associated therewith is beneficial to a subject, such as a human patient. The quality of life of a patient is improved by reducing to any degree the severity of symptoms in a subject and/or delaying the appearance of symptoms. Accordingly, the method in one aspect is performed as soon as possible after it has been determined that a subject is suffering from or at risk of suffering from MG, in particular generalised MG.

In various aspects, the antibody or fragment thereof is administered via an administration regimen that achieves an improvement in QMG, MG-Composite or MG-ADL score compared to pre-treatment (baseline). The improvement in score may be observed at, e.g., one, two, three, four, five, six, seven, eight, nine, ten, eleven, or twelve weeks following initial administration of the antibody or antigen-binding fragment thereof. In one example the improvement in score compared to baseline is observed at day 29 or day 43 or day 50 after initial administration of the antibody or antigen-binding fragment thereof.

Improvements are as set out above for clinical response, for example an improvement may be a ≥3.0 point improvement from Baseline score, such as a ≥3.0 point improvement from Baseline QMG score, MG-Composite and/or MG-ADL score.

In one example an improvement may be a ≥3.0 point improvement from Baseline score, such as a ≥3.0 point improvement from Baseline QMG score and/or MG-Composite and/or MG-ADL score.

In one example an improvement may be a ≥3.0 point improvement from Baseline score, such as a ≥3.0 point improvement from Baseline QMG score and/or a ≥3.0 point improvement from Baseline MG-Composite score and/or a ≥2.0 point improvement from Baseline MG-ADL score.

In one example the MG-ADL score is improved compared to baseline at day 43 i.e. the MG-ADL score is decreased by at least 2 points, for example at least 3 points.

In one example the MG-Composite score is improved compared to baseline at day 43 i.e. the MG-Composite score is decreased by at least 3 points. In one example the MG-Composite score is reduced by at least 4, 5 or 6 points.

In one example the QMG score is improved compared to baseline at day 43 i.e. the QMG score is decreased by at least 2 points, for example at least 3 points.

Alternatively or in addition, the antibody or fragment thereof is administered via an administration regimen that achieves a reduction in IgG serum levels and/or a reduction in MG-specific autoantibody (anti-MuSK and/or anti-AChR antibody) serum levels compared to pre-treatment (baseline). The reduction in levels may be observed at, e.g., one, two, three, four, five, six, seven, eight, nine, ten, eleven, or twelve weeks following initial administration of the antibody or antigen-binding fragment thereof. In one example the reduction in serum levels is observed at day 29 or day 43 or day 50 after initial administration of the antibody or antigen-binding fragment thereof. The IgG serum levels and/or MG-specific autoantibody (anti-MuSK and/or anti-AChR antibody) serum levels may be reduced by at least 50%, at least 55%, at least 60%, at least 65% or at least 70%, in particular compared to baseline.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. Generally, a therapeutically effective amount will be from 4 mg/kg to 50 mg/kg (e.g., 4 mg/kg to 25 mg/kg, such as about 7 mg/kg, 10 mg/kg, 15 mg/kg or 20 mg/kg). Compositions may be conveniently presented in unit dose forms containing a predetermined amount of an active agent of the disclosure per dose. Dose ranges and regimens for any of the embodiments described herein include, but are not limited to, dosages ranging from 1 mg-1000 mg unit doses (such as 100 mg, 140 mg, 160 mg unit doses given every 1-10 weeks (by any route of administration, such as by either a subcutaneous or intravenous administration). Further suitable unit doses may be doses in the range from 250-1250 mg, for example a dose selected from 280 mg, 420 mg, 560 mg, 840 mg and 1120 mg. Examples of other suitable unit doses may be a dose selected from 280 mg, 315 mg, 350 mg, 385 mg, 420 mg, 455 mg, 490 mg, 525 mg, 560 mg, 595 mg, 630 mg, 665 mg, 700 mg, 735 mg, 770 mg, 805 mg, 840 mg, 875 mg, 910 mg, 945 mg, 980 mg, 1015 mg, 1050 mg, 1085 mg and 1120 mg.

Accordingly, the present invention provides a method of treating or preventing myasthenia gravis (MG) in a human in need thereof, the method comprising administering to the human at least three doses, preferably at least six doses of an anti-FcRn antibody or antigen binding fragment thereof wherein each dose is independently selected from 4 mg/kg, 7 mg/kg, 10 mg/kg, 15 mg/kg and 20 mg/kg.

As set out above, fixed unit dosing may also be used. In one example the present invention also provides a method of treating or preventing myasthenia gravis (MG) in a human in need thereof, the method comprising administering to the human at least three doses, preferably at least six doses, of an anti-FcRn antibody or antigen-binding fragment thereof wherein each dose is independently selected from 280 mg, 420 mg, 560 mg, 840 mg and 1120 mg and wherein the dose is optionally is selected based on the weight of the patient.

In one example the present invention also provides a method of treating or preventing myasthenia gravis (MG) in a human in need thereof, the method comprising administering to the human at least three doses, preferably at least six doses, of an anti-FcRn antibody or antigen-binding fragment thereof wherein each dose is selected from 280 mg, 315 mg, 350 mg, 385 mg, 420 mg, 455 mg, 490 mg, 525 mg, 560 mg, 595 mg, 630 mg, 665 mg, 700 mg, 735 mg, 770 mg, 805 mg, 840 mg, 875 mg, 910 mg, 945 mg, 980 mg, 1015 mg, 1050 mg, 1085 mg and 1120 mg and wherein the anti-FcRn antibody or antigen binding fragment thereof optionally comprises a heavy chain comprising the sequence given in SEQ ID NO:29 and a light chain comprising the sequence given in SEQ ID NO:15. Again, the dose may be selected based on the weight of the patient.

In one example fixed unit doses across body weight tiers may be employed.

For example 6 subcutaneous doses at 1 week intervals (equivalent to approximately 7 mg/kg)
Body weight <50 kg: dose to be administered 280 mg
Bodyweight ≥50 kg and <70 kg: dose to be administered 420 mg
Bodyweight ≥70 kg and <100 kg: dose to be administered 560 mg
Bodyweight ≥100 kg; dose to be administered 840 mg

### Or

For example 6 subcutaneous doses at 1 week intervals (equivalent to approximately 10 mg/kg)
Body weight <50 kg: dose to be administered 420 mg
Bodyweight ≥50 kg and <70 kg: dose to be administered 560 mg
Bodyweight ≥70 kg and <100 kg: dose to be administered 840 mg
Bodyweight ≥100 kg: dose to be administered 1120 mg

In one example a fixed unit dose equivalent to approximately 7 mg/kg is used, in one example for a body weight of less than 50 kg the dose is 280 mg. In one example for a body weight of equal to or greater than 50 kg but less than 70 kg the dose is 420 mg. In one example for a body weight of equal to or greater than 70 kg but less than 100 kg the dose is 560 mg. In one example for a body weight of equal to or greater than 100 kg the dose is 840 mg.

Accordingly, in one example the present invention also provides a method of treating or preventing myasthenia gravis (MG) in a human in need thereof, the method comprising administering to the human at least 3 doses, preferably at least 6 doses, of an anti-FcRn antibody or antigen-binding fragment thereof wherein for a body weight of less than 50 kg the dose is 280 mg, for a body weight of equal to or greater than 50 kg but less than 70 kg the dose is 420 mg, for a body weight of equal to or greater than 70 kg but less than 100 kg the dose is 560 mg and for a body weight of equal to or greater than 100 kg the dose is 840 mg.

In one example a fixed unit dose equivalent to approximately 10 mg/kg is used, in one example for a body weight of less than 50 kg the dose is 420 mg. In one example for a body weight of equal to or greater than 50 kg but less than 70 kg the dose is 560 mg. In one example for a body weight of equal to or greater than 70 kg but less than 100 kg the dose is 840 mg. In one example for a body weight of equal to or greater than 100 kg the dose is 1120 mg.

Accordingly, in one example the present invention also provides a method of treating or preventing myasthenia gravis (MG) in a human in need thereof, the method comprising administering to the human at least 3 doses, preferably at least 6 doses, of an anti-FcRn antibody or antigen-binding fragment thereof wherein for a body weight of less than 50 kg the dose is 420 mg, for a body weight of equal to or greater than 50 kg but less than 70 kg the dose is 560 mg, for a body weight of equal to or greater than 70 kg but less than 100 kg the dose is 840 mg and for a body weight of equal to or greater than 100 kg the dose is 1120 mg.

In one example a fixed unit dose for a single body weight tier of above or below 100 kg is used. In one example a fixed unit dose for a body weight of less than 100 kg is selected from 280 mg, 315 mg, 350 mg, 385 mg, 420 mg, 455 mg, 490 mg, 525 mg, 560 mg, 595 mg, 630 mg, 665 mg, 700 mg, 735 mg, 770 mg, 805 mg, 840 mg, 875 mg, 910 mg, 945 mg, 980 mg, 1015 mg, 1050 mg, 1085 mg and 1120 mg. In one example a fixed unit dose for a body weight of less than 100 kg is 420 mg or 560 mg or 840 mg.

Accordingly, in one example the present invention also provides a method of treating or preventing myasthenia gravis (MG) in a human in need thereof, the method comprising administering to the human at least 3 doses, preferably at least 6 doses, of an anti-FcRn antibody or antigen-binding fragment thereof wherein for a body weight of less than 100 kg the dose is selected from 280 mg, 315 mg, 350 mg, 385 mg, 420 mg, 455 mg, 490 mg, 525 mg, 560 mg, 595 mg, 630 mg, 665 mg, 700 mg, 735 mg, 770 mg, 805 mg, 840 mg, 875 mg, 910 mg, 945 mg, 980 mg, 1015 mg, 1050 mg, 1085 mg and 1120 mg.

In one example a fixed unit dose for a body weight of equal to or greater than 100 kg is selected from 280 mg, 315 mg, 350 mg, 385 mg, 420 mg, 455 mg, 490 mg, 525 mg, 560 mg, 595 mg, 630 mg, 665 mg, 700 mg, 735 mg, 770 mg, 805 mg, 840 mg, 875 mg, 910 mg, 945 mg, 980 mg, 1015 mg, 1050 mg, 1085 mg and 1120 mg.

In one example a fixed unit dose for a body weight of equal to or greater than 100 kg is 840 mg or 1120 mg.

Accordingly, in one example the present invention also provides a method of treating or preventing myasthenia gravis (MG) in a human in need thereof, the method comprising administering to the human at least 3 doses, preferably at least 6 doses, of an anti-FcRn antibody or antigen-binding fragment thereof wherein for a body weight of equal to or greater than 100 kg the dose is selected from 280 mg, 315 mg, 350 mg, 385 mg, 420 mg, 455 mg, 490 mg, 525 mg, 560 mg, 595 mg, 630 mg, 665 mg, 700 mg, 735 mg, 770 mg, 805 mg, 840 mg, 875 mg, 910 mg, 945 mg, 980 mg, 1015 mg, 1050 mg, 1085 mg and 1120 mg.

In one example the present invention also provides a method of treating or preventing myasthenia gravis (MG) in a human in need thereof, the method comprising administering to the human at least 3 doses, preferably at least 6 doses, of an anti-FcRn antibody or antigen-binding fragment thereof wherein for a body weight of less than 100 kg the dose is 420 mg or 560 mg or 840 mg and for a body weight of equal to or greater than 100 kg the dose is 840 mg or 1120 mg.

In one example the present invention also provides a method of treating or preventing myasthenia gravis (MG) in a human in need thereof, the method comprising administering to the human at least 3 doses, preferably at least 6 doses, of an anti-FcRn antibody or antigen-binding fragment thereof wherein for a body weight of less than 100 kg the dose is 420 mg or 560 mg and for a body weight of equal to or greater than 100 kg the dose is 840 mg or 1120 mg.

In one example, a dose of antibody or antigen-binding fragment thereof is administered every week, for example every week for at least 3 weeks. In one example, a dose of antibody is administered every week for at least five or six weeks. The treatment period (i.e., the period of time during which one or more doses of antibody are administered to a subject) may comprise at least three weeks, at least four weeks, at least five weeks, at least six weeks, at least seven weeks, at least eight weeks or more. Any suitable number of doses may be administered within the treatment period, such as the doses and time between administrations described herein. For example, six doses of antibody may be administered to a subject over, e.g. a 5 week treatment period (week 0, week 1, week 2, week 3, week 4 and week 5).

Thus in one aspect there is provided a method of treating or preventing myasthenia gravis (MG) in a human in need thereof, the method comprising administering to the human at least 3 doses of an anti-FcRn antibody or antigen-binding fragment thereof wherein each dose is independently selected from 4 mg/kg, 7 mg/kg, 10 mg/kg, 15 mg/kg and 20 mg/kg.

In one example each dose is 4 mg/kg, for example administered as six individual doses, in particular six individual doses over six consecutive weeks.

In one example each dose is 7 mg/kg, for example administered as six individual doses, in particular six individual doses over six consecutive weeks.

In one example each dose is 10 mg/kg, for example administered as six individual doses, in particular six individual doses over six consecutive weeks.

In one example each dose is 15 mg/kg, for example administered as six individual doses, in particular six individual doses over six consecutive weeks.

In one example each dose is 20 mg/kg, for example administered as six individual doses, in particular six individual doses over six consecutive weeks.

As set out above, fixed unit dosing may also be employed, and for example each dose may be a dose selected from 280 mg, 315 mg, 350 mg, 385 mg, 420 mg, 455 mg, 490 mg, 525 mg, 560 mg, 595 mg, 630 mg, 665 mg, 700 mg, 735 mg, 770 mg, 805 mg, 840 mg, 875 mg, 910 mg, 945 mg, 980 mg, 1015 mg, 1050 mg, 1085 mg and 1120 mg, administered as six individual doses, in particular six individual doses over six consecutive weeks.

In one example each dose is 280 mg, for example administered as six individual doses, in particular six individual doses over six consecutive weeks.

In one example each dose is 420 mg, for example administered as six individual doses, in particular six individual doses over six consecutive weeks.

In one example each dose is 560 mg, for example administered as six individual doses, in particular six individual doses over six consecutive weeks.

In one example each dose is 840 mg, for example administered as six individual doses, in particular six individual doses over six consecutive weeks.

In one example each dose is 1120 mg, for example administered as six individual doses, in particular six individual doses over six consecutive weeks.

As described herein above, the fixed unit dosing may be based on weight tiers, with the selected dose based on weight tier being administered for example weekly for at least six consecutive weeks. As described above, for example a 280 mg dose is used for a weight of less than 50 kg.

In one example the method employs a dosing holiday between the third and fourth doses. In one example the dosing holiday is 1 week. In one example the dosing holiday is 2 weeks.

Optionally, the method employs a repeat dose administration strategy with different dosing regimens involving a higher initial dose (i.e., a “loading dose”) followed by one or more lower doses (i.e., one or more second or additional doses that are lower than the initial dose (“maintenance doses”)), although a lower loading dose followed by higher maintenance doses also are contemplated. In one embodiment, the maintenance doses may be one-quarter, one-third, one-half, two-thirds, three-quarters, the same as, one and one-quarter, one and one-third, one and one-half, one and two-thirds, one and three-quarters, double, or more of the loading dose. A multiple dose regimen without a loading dose also is contemplated as part of the disclosure.

In one embodiment, one or more maintenance doses are administered at an interval after administration of a loading dose. This interval may be consistent for each dose or may vary. This interval may be 1 day, 1 week, 2 weeks, 3 weeks, 4 weeks, monthly, 6 weeks, 8 weeks, every other month, or at any other interval.

Accordingly, in one example, the method of treatment of the present invention further comprises administering one or more second or additional doses that are lower than the initial dose(s).

It will be appreciated that these additional doses may be administered beyond the initial treatment period for the higher ‘loading dose’.

Accordingly, in one example, the initial dosing over a treatment period of preferably 4-6 weeks, may be followed by a further maintenance dosing treatment period at a lower dose and/or lower frequency of dosing.

In one example each dose is 7 mg/kg, for example administered as six individual doses over six consecutive weeks, optionally followed by one more additional doses.

In one example each dose is 10 mg/kg, for example administered as six individual doses over six consecutive weeks, optionally followed by one more additional doses.

In one example, the additional doses are lower “maintenance” doses. In one example, each lower (“maintenance”) dose is between 4 and 10 mg/Kg, preferably 4 or 7 mg/Kg.

In one example, each lower (“maintenance”) dose is between 4 and 20 mg/Kg, preferably 7 or 10 mg/Kg, given less frequently than the initial “loading” doses, e.g. less frequently than weekly e.g. every 2 or 4 weeks or monthly.

As set out above, these maintenance doses may be given at any suitable interval such as 1 week, 2 weeks, 3 weeks, 4 weeks, monthly, 6 weeks, 8 weeks, every other month, or at any other interval.

In one example a maintenance dose is given at an interval of every week.

In one example a maintenance dose is given at an interval of every 2 weeks.

In one example a maintenance dose is given at an interval of every 4 weeks.

In one example a maintenance dose is given monthly.

In one example the higher initial dose (loading dose) comprises treatment with 6 doses of 4 mg/Kg or 7 mg/Kg or 10 mg/kg or 15 mg/kg or 20 mg/kg at weekly intervals over a first treatment period and maintenance dosing may comprise dosing at 4 mg/Kg or 7 mg/Kg at a suitable interval such every 2 weeks, 3 weeks, 4 weeks, monthly, 6 weeks, 8 weeks, every other month, or at any other interval.

In one example the higher initial dose (loading dose) comprises treatment with 6 doses of 4 mg/Kg or 7 mg/Kg or 10 mg/kg or 15 mg/kg or 20 mg/kg at weekly intervals over a first treatment period and maintenance dosing may comprise dosing at 4 mg/Kg or 7 mg/Kg or 10 mg/Kg at a suitable interval such as every 2 weeks, 3 weeks, 4 weeks, monthly, 6 weeks, 8 weeks, every other month, or at any other interval.

In one example the higher initial dose (loading dose) comprises treatment with 6 doses of 10 mg/kg at weekly intervals over a first treatment period of six weeks and maintenance dosing may comprise dosing at 7 mg/kg at a suitable interval such as every week, every 2 weeks, 3 weeks, 4 weeks, monthly, 6 weeks, 8 weeks, every other month, or at any other interval.

In one example the higher initial dose (loading dose) comprises treatment with 6 doses of 10 mg/kg at weekly intervals over a first treatment period of six weeks and maintenance dosing may comprise dosing at 10 mg/kg at a suitable less frequent interval such as every 2 weeks, 3 weeks, 4 weeks, monthly, 6 weeks, 8 weeks, every other month, or at any other interval.

As set out above, fixed unit dosing may also be used in the present invention, optionally based on body weight tiers as described herein above. In one example the higher initial dose (loading dose) comprises treatment with 6 doses of 280 mg or 420 mg or 560 mg or 840 mg or 1120 mg, optionally based on body weight tier, at weekly intervals over a first treatment period and maintenance dosing may comprise (i) dosing at a lower dose at a suitable interval such as every week, every 2 weeks, 3 weeks, 4 weeks, monthly, 6 weeks, 8 weeks, every other month, or at any other interval or (ii) dosing the same dose less frequently.

The method of treatment of the present invention may be suitable for both chronic-intermittent treatment and/or maintenance therapy.

It will be appreciated that frequency of dosing may be determined by disease severity, determined for example by disease biomarker monitoring and/or by monitoring patient MG-specific autoantibody (anti-MuSK and/or anti-AChR antibody) serum levels and/or serum IgG levels.

Timing between administrations may decrease as the condition improves or increase if it worsens, reverting to the higher doses as needed for acute episodes.

Timing of administration may also be determined by monitoring patient anti-AChR antibody titer and/or anti-MuSK titer and/or serum IgG levels.

Autoantibodies involved in the pathogenesis of MG may include both IgG and non-IgG isotypes. In one example, the method of treatment of the present disclosure may be used for the treatment of MG patients where IgG is determined to be the dominant isotype.

Comprising in the context of the present specification is intended to meaning including. Where technically appropriate embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

The present invention is further described by way of illustration only in the following examples, which refer to the accompanying Figures, in which:

FIG. 1 shows certain amino acid and polynucleotide sequences.

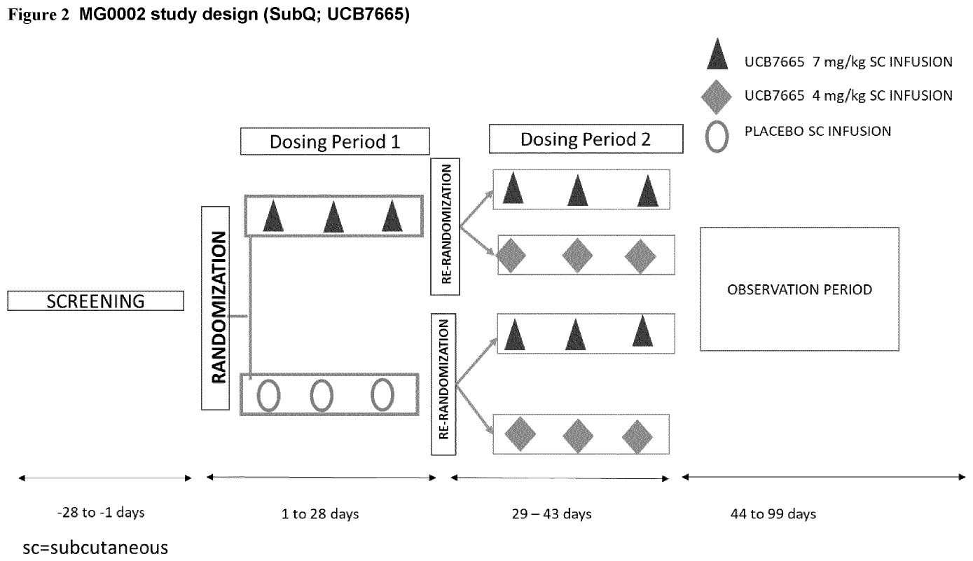

FIG. 2 MG0002 study design (SubQ; UCB7665)

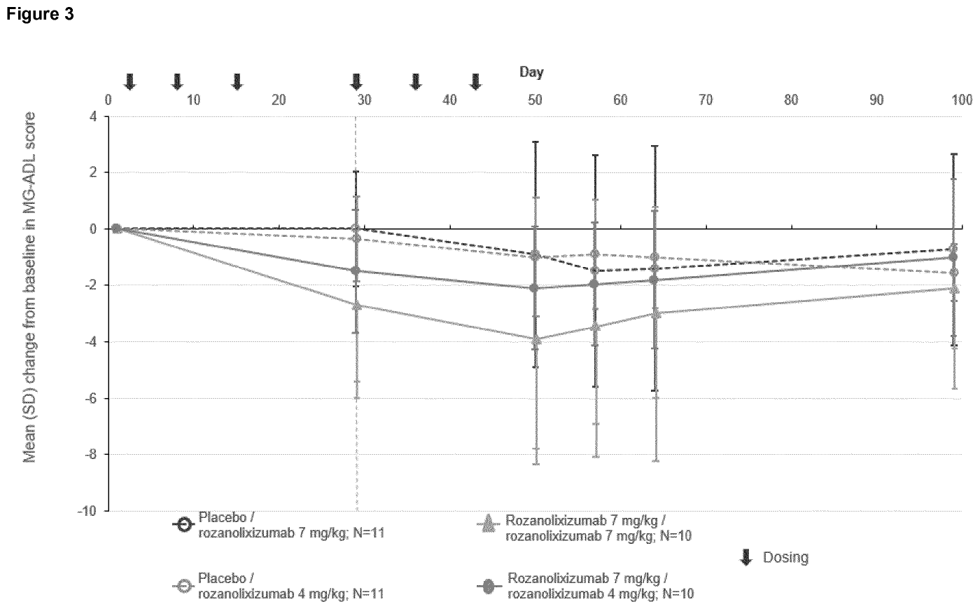

FIG. 3 Change from baseline in MG-ADL score (rozanolixizumab 7 mg/kg/rozanolixizumab 4 mg/kg) and (rozanolixizumab 7 mg/kg/rozanolixizumab 7 mg/kg)

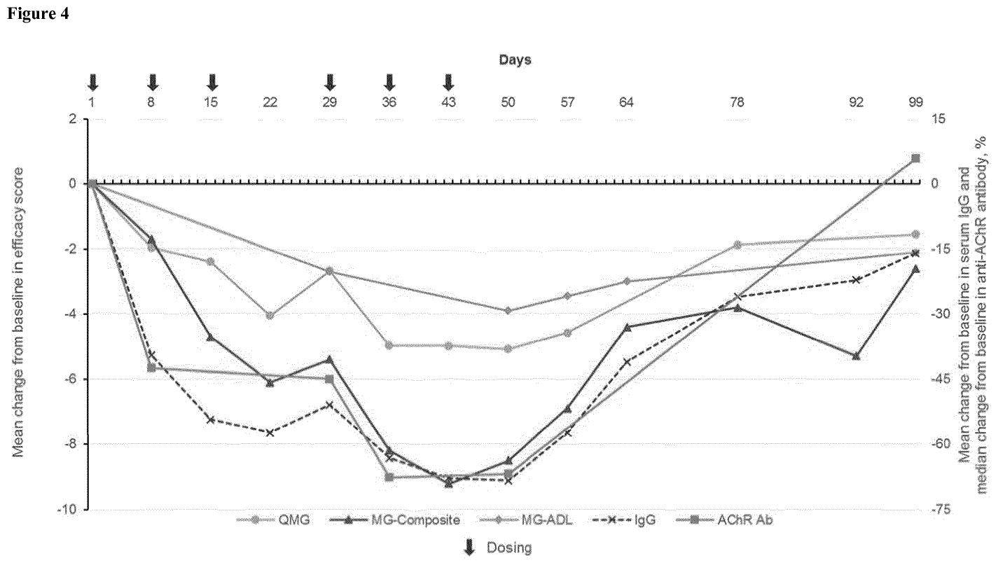

FIG. 4 Change from baseline in QMG, MG-Composite, MG-ADL scores, serum IgG concentration and anti-AChR antibody (rozanolixizumab 7 mg/kg/rozanolixizumab 7 mg/kg)

FIG. 5 Quantitative Myasthenia Gravis testing form

FIG. 6 Myasthenia Gravis Composite Score

FIG. 7 Myasthenia Gravis Activities of Daily Living (MG-ADL) Scoring

### EXAMPLES

### Example 1

UCB7665 was first described in WO2014019727 and comprises the CDR sequences provided herein in SEQ ID NOs 1-6. It comprises the light chain of SEQ ID NO:22 and the heavy chain of SEQ ID NO: 43.

UCB7665 has the INN rozanolixizumab.

Affinity for hFcRn Binding of UCB 7665 (Reproduced from WO2014019727)

Biomolecular interaction analysis using surface plasmon resonance technology (SPR) was performed on a Biacore T200 system (GE Healthcare) and binding to human FcRn extracellular domain determined. Human FcRn extracellular domain was provided as a non-covalent complex between the human FcRn alpha chain extracellular domain (SEQ ID NO:94) and β2 microglobulin (β2M) (SEQ ID NO:95). Affinipure F(ab′)₂ fragment goat anti-human IgG, F(ab′)₂ fragment specific (for Fab′-PEG capture) or Fc fragment specific (for IgG1 or IgG4 capture) (Jackson ImmunoResearch Lab, Inc.) in 10 mM NaAc, pH 5 buffer was immobilized on a CM5 Sensor Chip via amine coupling chemistry to a capture level between 4000-5000 response units (RU) using HBS-EP⁺ (GE Healthcare) as the running buffer. 50 mM Phosphate, pH6+150 mM NaCl+0.05% P20 or HBS-P, pH7.4 (GE Healthcare) was used as the running buffer for the affinity assay. The antibody was diluted to 4 μg/ml (IgG4) in running buffer. A 60 s injection of IgG4 at 10 μl/min was used for capture by the immobilized anti-human IgG, F(ab′)2. Human FcRn extracellular domain was titrated from 20 nM to 1.25 nM over the captured anti-FcRn antibody for 300 s at 30 μl/min followed by 1200 s dissociation. The surface was regenerated by 2×60 s 50 mM HCl at 10 μl/min. The data was analysed using T200 evaluation software (version 1.0).

pH 7.4

| 1519.g57 | ka (M⁻¹s⁻¹) | kd (s⁻¹) | KD (M) | KD (pM) |
|---|---|---|---|---|
| IgG4P | 3.68E+05 | 1.26E−05 | 3.43E−11 | 34 |

pH 6

| 1519.g57 | ka (M⁻¹s⁻¹) | kd (s⁻¹) | KD (M) | KD (pM) |
|---|---|---|---|---|
| IgG4P | 4.43E+05 | 1.00E−05 | 2.26E−11 | 23 |

Affinity data for anti-hFcRn 1519.g57 IgG4P at pH7.4 and pH6 (average of three experiments)

Crystallography and Binding Epitope of UCB7665 (Reproduced from WO2014019727)

The crystal structure of 1519g57 Fab′ and deglycosylated human FcRn extracellular domain (alpha chain extracellular domain (SEQ ID NO:94) in association with beta2 microglobulin SEQ ID NO:95) was determined, with the FcRn oligsaccharide excluded in order to facilitate crystallization. 1519.g57 Fab′ was reacted with 10-fold molar excess of N-ethyl maleimide to prevent formation of diFab′ and any existing diFab′ removed by SEC (S200 on Akta FPLC). Human FcRn extracellular domain was treated by PNGaseF to remove N-linked sugars. For this, the FcRn sample concentration was adjusted using PBS (pH7.4) to 5 mg/ml and a total volume of 1 ml. 200 units of PNGaseF (Roche) was added to this solution of human FcRn. This was incubated at 37° C. for ˜18 hours, following which the extent of deglycosylation was checked using SDS PAGE. Upon completion of the reaction the deglycosylated FcRn was buffer exchanged into 50 mM Sodium Acetate, 125 mM NaCl, pH6.0.

The complex was formed by incubation of a mixture of reagents (Fab′:FcRn::1.2:1, w/w) at room temperature for 60 minutes, and then purified using SEC (S200 using Akta FPLC). Screening was performed using the various conditions that were available from Qiagen (approximately 2000 conditions). The incubation and imaging was performed by Formulatrix Rock Imager 1000 (for a total incubation period of 21 days).

There was no obvious change in FcRn structure upon binding of 1519g57 Fab′ (comparing this complex with published structures of FcRn). From the crystal structure it the secondary structure content was calculated to be: α-helix 9.4%; β-sheet 45.2%; 3-10 turn 2.5%.

The residues interacting with 1519g57 Fab′ were all in the FcRn α chain (not β2M) and are indicated below in bold. The residues concerned encompass all but 1 of the residues critical for binding Fc. 1519g57 binds in a region that overlays the Fc-binding region, suggesting that blockade of FcRn by 1519g57 Fab′ is by simple competition, the anti-FcRn being effective by virtue of its superior affinity.

AESHLSLLYH LTAVSSPAPG TPAFWVSGWL GPQQYLSYNS
LRGEAEPCGA WVWENQVSWY WEKETTDLRI KEKLFLEAFK
ALGGKGPYTL QGLLGCELGP DNTSVPTAKF ALNGEEFMNF
DLKQGTWGGD WPEALAISQR WQQQDKAANK ELTFLLFSCP
HRLREHLERG RGNLEWKEPP SMRLKARPSS PGFSVLTCSA
FSFYPPELQL RFLRNGLAAG TGQGDFGPNS DGSFHASSSL
TVKSGDEHHY CCIVQHAGLA QPLRVELESPAKSS

The FcRn α chain sequence, showing residues involved in interaction with 1519g57 Fab′ (bold) and residues critical for interaction with Fc of IgG (underlined). All but 1 of the latter are included in the former.

### Example 2

The MG0002 study is a Phase 2a, multicenter, randomized, investigator- and subject-blind, placebo-controlled, 2-arm, repeat dose, treatment sequence study which will evaluate the efficacy, safety, and tolerability of chronic-intermittent treatment with UCB7665 in subjects with moderate to severe generalized myasthenia gravis (MG). UCB7665 will be administered as subcutaneous (sc) doses of 4 mg/kg or 7 mg/kg, in subjects ≥18 years of age.

The study is planned to be conducted at approximately 30 sites in United States of America (USA), Canada, and Europe, with possible extension to other regions and countries. A total of 42 subjects are planned to enter the Treatment Period in the study. The maximum study duration for an individual subject is approximately 18 weeks.

The study will consist of 3 Periods: Screening, Treatment, and Observation. After Screening, subjects will enter the Treatment Period, which will consist of Dosing Period 1 followed by Dosing Period 2. Subjects will receive 3 doses of investigational medicinal product (IMP) at weekly intervals during each dosing period as follows:

Dosing Period 1 will be 4 weeks, with 2 parallel arms (UCB7665 7 mg/kg or placebo).
Dosing Period 2 will be 2 weeks, with 2 parallel treatment arms (UCB7665 7 mg/kg or UCB7665 4 mg/kg).
The Observation Period will span 8 weeks after the final dose of UCB7665, with a Final Visit (FV) being scheduled at Visit 20. The sc infusions will last approximately 30 minutes, and subjects will be required to remain in the hospital/clinic at least 4 hours for safety monitoring after each infusion.

The primary efficacy variable will be the change from Baseline in Quantitative MG (QMG) score to Visit 9 (Day 29). The secondary efficacy variables will be the change from Baseline in MG-Composite score to Visit 9 (Day 29) and the change from Baseline in MG-Activities of Daily Living (MGADL) score to Visit 9 (Day 29). Other efficacy variables will be the following:

value and change from Baseline in QMG at each scheduled assessment during Treatment and Observation Periods; QMG responder (≥3.0 point improvement from Baseline) at each scheduled assessment during Treatment and Observation Periods; value and change from Baseline in MG-Composite score at each scheduled assessment during Treatment and Observation Periods;
MG-Composite responder (≥3.0 point improvement from Baseline) at each scheduled assessment during Treatment and Observation Periods; value and change from Baseline in MGADL at each scheduled assessment during Treatment and Observation Periods; MGADL responder (≥3.0 point improvement from Baseline) at each scheduled assessment during Treatment and Observation Periods; Myasthenia Gravis Foundation of America (MGFA) classification at each scheduled assessment during Treatment and Observation Periods; value and change from Baseline in MG muscle weakness severity, fatigue scales and Myasthenia Gravis Impairment Index (MGII) scores at each scheduled assessment during Treatment and Observation Periods;
and change in mean consecutive difference (MCD) in jitter (single fiber electromyography [SFEMG]) studies from Baseline to Visit 9 for the subjects consenting to this measurement at participating sites.

Other and exploratory variables include: safety and tolerability variables, pharmacokinetic (PK), pharmacodynamic (PD), and immunologic variables.

Safety and tolerability variables include the following: occurrence of treatment-emergent adverse events (TEAEs); vital sign values and changes from Baseline (systolic and diastolic blood pressure [BP], temperature, pulse rate, respiratory rate, and body weight); 12-lead electrocardiogram (ECG) values and change from Baseline; laboratory values (hematology, clinical chemistry, and urinalysis) and changes from Baseline; change from Baseline in exploratory safety biomarkers (may include but not limited to S100 calcium-binding protein B [S100B], neuron-specific enolase, prostaglandins and/or their metabolites, serotonin, and tryptase) in subjects experiencing severe headache and/or moderate to severe gastrointestinal (GI) disturbance; and TEAEs leading to withdrawal of IMP.

Plasma concentration of UCB7665 over time will be assessed as the PK variable. The main PD variables will be the maximum decrease from Baseline in serum total immunoglobulin G (IgG) concentration; value and change from Baseline in total serum IgG concentrations at each scheduled assessment during Treatment and Observation Periods; value and change from Baseline in serum IgG subclass concentrations at each scheduled assessment during Treatment and Observation Periods; and change in MG-specific autoantibody (anti-MuSK/anti-AChR) levels in serum from Baseline at each scheduled assessment during Treatment and Observation Periods. Additionally, change from Baseline in other immunological variables and other exploratory biomarkers during the Treatment and Observational Periods will be assessed.

### Study Variables

### 1 Efficacy Variables

### 1.1 Primary Efficacy Variable

The primary efficacy variable is:

Change from Baseline in QMG score to Visit 9 (Day 29)
1.2 Secondary efficacy variable

The secondary efficacy variables are:

Change from Baseline in MG-Composite score to Visit 9 (Day 29)
Change from Baseline in MGADL score to Visit 9 (Day 29)
1.3 Other efficacy variables

The other efficacy variables are:

Value and change from Baseline in QMG at each scheduled assessment during Treatment and Observation Periods
QMG responder (≥3.0 point improvement from Baseline) at each scheduled assessment during Treatment and Observation Periods
Value and change from Baseline in MG-Composite score at each scheduled assessment during Treatment and Observation Periods
MG-Composite responder (≥3.0 point improvement from Baseline) at each scheduled assessment during Treatment and Observation Periods
Value and change from Baseline in MGADL at each scheduled assessment during Treatment and Observation Periods
MGADL responder (≥3.0 point improvement from Baseline) at each scheduled assessment during Treatment and Observation Periods
MGFA classification at each scheduled assessment during Treatment and Observation Periods
Value and change from Baseline in MG muscle weakness and fatigability at each scheduled assessment during Treatment and Observation Periods
Value and change from Baseline in fatigue at each scheduled assessment during Treatment and Observation Periods
Value and change from Baseline in MGII scores at each scheduled assessment during Treatment and Observation Periods
Change in the percentage of normal fiber pairs in jitter (SFEMG) studies from Baseline to Visit 9 for the subjects consenting to this measurement at the participating sites Change in MCD of the interpotential interval (IPI) in jitter (SFEMG) studies from Baseline to Visit 9 for the subjects consenting to this measurement at the participating sites A reduction in MCD of ≥9 μs in jitter (SFEMG) studies will define clinical improvement

### Study Design

### Study Description

This is a Phase 2a, multicenter, randomized, investigator- and subject-blind, placebo-controlled, 2-arm, repeat dose, treatment sequence study evaluating the safety and efficacy of UCB7665 as an chronic-intermittent treatment for subjects with moderate to severe generalized MG.

Approximately 42 randomized subjects will be enrolled at approximately 30 sites from USA, Canada, and Europe to achieve the targeted number of 40 evaluable subjects.

The maximum duration of the study per subject is approximately 18 weeks, consisting of a Screening Period (1 to 28 days), Treatment Period (6 weeks), and an Observation Period (8 Weeks).

Screening Period: The purpose of the Screening Period is to evaluate and confirm the subject's eligibility. During the Screening Visit (Visit 1), subjects will sign a written Informed Consent form prior to the conduct of any study-related procedures. The use of concomitant medication while in the study will be discussed and subjects' eligibility will be determined on the basis of the inclusion/exclusion criteria. The Screening Period should not exceed 28 days in total.

Treatment Period: The Treatment Period will consist of Dosing Period 1 followed by Dosing Period 2 (See FIG. 2). Subjects will receive 3 doses of IMP at weekly intervals during each dosing period as follows.

Dosing Period 1 will be 4 weeks, with 2 parallel Treatment Groups (UCB7665 7 mg/kg or placebo).

Dosing Period 2 will be 2 weeks, with 2 parallel Treatment Groups (UCB7665 7 mg/kg or UCB7665 4 mg/kg).

Prior to receiving an infusion with IMP, subjects will be assessed for efficacy measurements at each visit in the Treatment Period. For all safety and efficacy measurements, the order specified in the Study Procedures Manual is recommended to be used as a guide.

Dosing Period 1: Dosing Period 1 will last for approximately 4 weeks (Day 1 to Day 28) and includes Visits 2, 3, 4, 5, 6, 7, and 8. Following completion of the Screening Period, eligible subjects will check-in at the clinic/hospital for the Randomization Visit (Visit 2). Subjects who continue to meet eligibility requirements will be randomized 1:1 to receive 7 mg/kg of UCB7665 or placebo, administered by an approximately 30 minute sc infusion at weekly intervals for 3 weeks (Visits 2, 4, and 6) followed by an assessment visit at Week 4 (Visit 8). At Visits 2, 4, and 6 in Dosing Period 1, subjects will be required to remain in the clinic/hospital for at least 4 hours for safety monitoring after the infusion. Subjects may leave the clinic/hospital once the safety monitoring postdose period is over and the investigator or designee has no safety concerns. A follow-up telephone call will be conducted 24 hours postdose to assess the status of the subject (Visits 3, 5, and 7). Subjects will return to the clinic/hospital at Visit 8 for safety and efficacy assessments. The primary efficacy endpoint, change from Baseline in the QMG score, will be assessed at the beginning of Dosing Period 2 at Visit 9 (Day 29) prior to rerandomization. The efficacy assessments that are performed at Visit 9 will therefore occur 2 weeks after the final dose of study drug in Dosing Period 1.

Dosing Period 2: Dosing Period 2 will last for approximately 2 weeks (Day 29 to Day 43) and includes Visit 9, 10, 11, 12, 13, and 14. Subjects will return to the clinic for Visit 9, 11, and 13 for safety and efficacy assessments

At Visit 9, following the administration of safety and efficacy assessments, subjects initially randomized at Baseline to placebo or to 7 mg/kg of UCB7665 willbe rerandomized 1:1 to receive either 3 doses of 7 mg/kg or 3 doses of 4 mg/kg administered by a 30 minute sc infusion at weekly intervals (Visit 9, 11, and 13). The interactive response technology (IRT) will stratify the rerandomization based on the treatment received in Dosing Period 1. At each weekly clinic visit in Dosing Period 2 (Visits 9, 11, and 13), subjects will be required to remain in the clinic/hospital for at least 4 hours safety monitoring postdose period as determined. Subjects may leave the clinic/hospital once the safety monitoring postdose period is over and the investigator or designee has no safety concerns. A follow-up telephone call will be conducted 24 hours postdose to assess the status of the subject (Visits 10, 12, and 14).

Observation Period: All subjects must be followed for 8 weeks after the final dose of IMP is administered. Subjects will return to the clinic for Visits 15, 16, 18, and 20 for efficacy and for safety assessments. Subjects will either return to the clinic/hospital, or, if possible and agreed by both investigator and subject, have home visits conducted by certified healthcare professionals, for Visits 17 and 19. The Observation Period begins the day after the final dose of IMP (ie, Visit 13, Day 43); Visit 15 (Day 50) is the first visit in the Observation Period.

### Inclusion Criteria

To be eligible to participate in this study, all of the following criteria must be met:

1. An Institutional Review Board (IRB)/Independent Ethics Committee (IEC) approved written Informed Consent form is signed and dated by the subject.

2. Subject is ≥18 years of age at Visit 1 (Screening).

3. Subject has a well-documented diagnosis of MG at Visit 1 (Screening), based on subject history and supported by previous evaluations.

4. Subject would currently be considered for treatment with immunological therapy (eg IVIG/PLEX) by the investigator.

5. Subject has a well-documented record of autoantibodies against AChR or MuSK prior to Visit 1 (Screening).

6. Female subjects of child bearing potential must have a negative serum pregnancy test at the Screening Visit, which is confirmed to be negative by urine testing prior to the first dose of study drug at Week 1 (Visit 2) and prior to further dosing at each study visit thereafter. Female subjects of childbearing potential must agree to use a highly effective method of birth control, during the study and for a period 2 months after their final dose of study drug.

According to the International Council for Harmonisation (ICH) M3 (R2), highly effective forms of birth control are methods which achieve a failure rate of less than 1% per year when used consistently and correctly. Highly effective methods of birth control include: Combined (estrogen- and progesterone-containing) hormonal contraception (oral, implant, injectable) associated with inhibition of ovulation, (which must be stable for at least 1 full month prior to Screening [Visit 1], and should remain stable during the study). Progesterone-only hormonal contraceptives (oral, implant, injectable) associated with inhibition of ovulation (which must be stable for at least 1 full month prior to Screening [Visit 1], and should remain stable during the study).

Progesterone-releasing intrauterine systems or the TCu 380A intrauterine device.

Vasectomized partner (provided sole partner and partner has medical proof of surgical success).

True heterosexual sexual abstinence is an acceptable form of contraception when this is in line with the preferred and usual lifestyle of the person. Periodic abstinence (eg, calendar, ovulation, symptothermal, postovulation methods), declaration of abstinence for the duration of the study, and withdrawal are not acceptable methods of contraception. Women not agreeing to use birth control must be of nonchildbearing potential, defined as being: Postmenopausal (for at least 2 years before the Screening Visit), verified by serum follicle stimulating hormone level >40 mIU/mL at the Screening Visit, or Permanently sterilized (eg, bilateral tubal occlusion, hysterectomy, bilateral salpingectomy), or □ Congenitally sterile

7. Contraception methods for male subjects and their female partners:

Male subject with a partner of childbearing potential must be willing to use a condom when sexually active during the study and for 3 months after the final administration of IMP.

In addition the female partner of childbearing potential of a male subject must be willing to use a highly effective method of contraception (as above), during the study period and for 3 months after the final administration of IMP.

### 6.2 Exclusion Criteria

Subjects are not permitted to enroll in the study if any of the following criteria are met:

1. Subject has previously received treatment in this study or subject has previously been exposed to UCB7665.

2. Subject has participated in another study of an IMP (or a medical device) within the previous 30 days of Screening or is currently participating in another study of an IMP (or a medical device).

3. Subject has a known hypersensitivity to any components of the IMP.

4. Subject has a history of hyperprolinemia, since L-proline is a constituent of the UCB7665 IMP.

### Concomitant Medication/Treatment

### Permitted Concomitant Medication

Concomitant medications that are permitted during the course of the study at a stable dose are Dose Comment

### Oral Corticosteroids

(eg, prednisolone)
Stable for 2 weeks prior to

### Baseline

Methotrexate ≤30 mg/week Treated for previous 6 months and
on stable dose for 2 months prior

### to Baseline

Mycophenolate mofetil ≤3 g/day Treated for previous 6 months and
on stable dose for 2 months prior

### to Baseline

Cyclosporina ≤5 mg/kg/day for
unmodified
≤4 mg/kg/day for modified
(microemulsion)
Treated for previous 6 months and
on stable dose for 2 months prior

### to Baseline

Azathioprine ≤3 mg/kg/day Treated for previous 6 months and
on stable dose for 2 months prior

### to Baseline

### Cholinesterase

inhibitors
≤600 mg

### Pyridostigmine/day

Stable dose not required—dose
held on days of efficacy outcomes
Tacrolimusb ≤5 mg/day Treated for previous 6 months and
on stable dose for 2 months prior

### to Baseline

a Doses higher than listed are permissible if trough level is ≤300 ng/L.
b If the total daily weight-based dose is >5 mg, then a plasma trough level should be checked to
ensure subject is not above the recommended therapeutic range.

Subjects should not take pyridostigmine (or any acetylcholinesterase inhibitor medication) from midnight before testing when medically safe to do so to standardize testing, but if not medically appropriate, then the treatment can be continued but the testing should be performed as best as possible at the same timeframe post any last acetylcholinesterase inhibitor medication inhibitor dosing for each evaluation during the study.

### Assessment of Efficacy

### 1. Quantitative Myasthenia Gravis Scale

For assessment of the QMG scale, investigators will follow the MGFA's QMG Manual instructions, as set out below under Quantative Myasthenia Gravis Testing form (see FIG. 5). Clinical personnel must complete mandatory training and be certified to assess subjects' QMG score (details are provided in the Study Procedures Manual).

Subjects should not take pyridostigmine (or any AChE inhibitor medication) from midnight before testing when medically safe to do so to standardize testing, but if not medically appropriate, then the treatment can be continued but the testing should be performed as best as possible at the same timeframe post last AChE inhibitor dosing for each evaluation during the study.

The scale tests 13 items, including ocular and facial involvement, swallowing, speech, limb strength, and forced vital capacity (FVC). For the assessment of FVC, the same spirometer should be used each time a subject is tested, and if possible, the same person should carry out the assessment. Parameters and normal values for FVC will be decided between the study sites, such that all sites are using the same information. The QMG is a validated assessment (Barnett et al, 2012), with a higher score indicating more severe disease. Scoring for each item ranges from no weakness (0) to severe weakness (3), with an overall score range from 0 to 39. A 3 point change in the total score is considered clinically relevant. The test takes approximately 30 minutes to perform.

### General Instructions

1. Patients must be off pyridostigmine (or any acetylcholinesterase inhibitor medication) for twelve (12) hours prior to testing, (if medically safe to do so).

2. Perform the tests in the order given in this Manual and shown on the Videotape.

3. Calibrate the respiratory equipment on the day of the test, per manufacturers' instruction, before the test begins. Place the calibration record in folder in an accessible place.

4. For all measurements, record actual numbers as well as grade, i.e. if it takes 30 seconds before a patient sees double, record on the far right box 30/1 for 30 seconds and a grade of 1.

5. Patients must remain seated for the respiratory test.

6. At the end of the scoring sheet, add up the grade for that patient and that becomes the Total QMG Score.

### Quantitative MG Score

### I. Double Vision:

Patients' preparation: Patient is sitting. Ask if the patient is experiencing double vision looking straight ahead. If yes, record 0/3 (actual time/grade) on the scoring sheet. If no, ask the patient to look to the right for just an instant and then to the left without moving their head. If the patient sees double in only one direction, record side and record result as 0/3. If there is no eye movement, record as 0/3. If the patient does not see double, or sees double in both directions, have them perform the test as described below gazing to the right.

Explanation to patient: “I need for you to face forward. When I ask, look over to your right (left) side without turning your head. If or when you start to see double, please let me know.” Notes to examiner: Patient's head will usually start to turn in the direction of the gaze. Try to maintain the head in a forward position. Record the time and grade. Example: double vision is evident at 15 sec. In the scoring section, record 15/1.

### II. Ptosis (Upward Gaze):

Patients' preparation: Patient is sitting. Ask the patient to look straight ahead. If the upper lid is touching the pupil, record as 0/3. Ask the patient to look up at the ceiling without moving the head.

Explanation to patient: “I need you to face forward. When I ask, look up at the ceiling without moving your head. Keep looking up until I tell you to relax.”

Notes to examiner: Patient's head will usually start to move up. Try to maintain the head in a forward position. Record time and grade when you see either eyelid (lashes) start to droop.

Ex: Right eyelid starts to droop at 9 sec., record 9/2. If neither eyelid touches the pupil, record 60/0.

### III. Facial Muscles:

Patients' preparation: Patient is sitting facing forward.

Explanation to patient: “Squeeze your eyes shut. Do not allow me to open your eyes.” Notes to examiner: If the patient cannot fully close either eye shut, record the grade as 3. No time score is needed on this test. Record grade of the weaker eye.

### IV. Swallowing:

Patient's preparation: Patient is sitting. Four ounces of water (no ice) is poured into a cup. The water should be no cooler than water fountain cool.

Explanation to patient: “I need for you to drink this water as you normally would.”

Notes to examiner: Listen for coughing and/or throat clearing during the test and immediately post test. Don't ask patients to drink faster than what they feel comfortable doing.

### V. Speech:

Patient's preparation: Patient is sitting.

Explanation to patient: “Count out loud from 1 to 50 at a comfortable pace.”

Notes to examiner: This is one of the most difficult tests to score because of varying accents. Record number when you notice a nasal or slurring of the speech.

### VI. Right & Left Arm Outstretched:

Patient's preparation: The patient needs to be sitting in a chair with both feet on the floor. They must be seated without leaning against the back of a chair. Test both arms at the same time.

Arms need to be out to the side at 90o, palms down. (Demonstrate this position). If the patient cannot raise an arm out to 90o due to a shoulder problem, do not test that arm. The elbows are extended through full mechanical range.

Explanation to patient: “I need for you to hold both arms out to the side like this. Keep the arms out as long as possible. If one arm tires more than the other, you may lower that arm and keep the other arm up.”

Notes to examiner: It is not uncommon that the arms start to droop. If the arms drop more than

10o from starting position, remind the patient to pull the arms up. If the patient can pull the arms up but cannot maintain that position for longer than two seconds, stop the test. If one arm is lowered, be careful that the patient does not start to lean to the side that the arm was lowered to give the appearance that he/she is maintaining a 90o angle. Record time/grade (ex: 45 sec for right arm is 45/2; whereas 100 sec for left arm is 100/1).

VII: Forced Vital Capacity: Patient Preparation: Patients Must Remain Seated for this Test.

Explanation to patient: “I am testing total lung capacity. I am going to ask you to hold this mouthpiece away from your face. I will then place the nose-clips on your nose. I will tell you to take a deep breath in, and then place the mouthpiece in your mouth. You will blow out as hard and as fast as you can. Keep blowing until I tell you to stop.

Notes to examiner: We are only testing FVC. A minimum of three trials and a maximum of five trials will be performed. The goal is to get the best two trials within 5% of each other. Give a lot of encouragement. Record best FVC (liters and percentage) and grade on sheet, (ex: 2.55-60%/2).

The “normal” FVC values, and therefore the percent predicted calculations can vary with the spirometer that is used. Some spirometers come with specified normal values. That is why the same spirometer should be used each time you test a subject. For multi-site studies, parameters and normal values should be decided so that all sites are using the same information.

### VIII: Right & Left Hand Grip:

Patient preparation: Patient is sitting in a chair. The elbow should be at 90o. Support should be under the medial aspect of the forearm and under the dynamometer.

Explanation to patient: “I am testing grip strength. I need for you to squeeze as hard as you can. Nothing will move, but it is measuring how hard you are squeezing.”

Notes to examiner: Give vocal encouragement. Record the two trials (kgs) in column and score (ex: if testing a female and results are 10 and 8 kgs, record as 10/1)

### IX. Head Lifted:

Patient preparation: The patient will lie down without a pillow under the head. A pillow may be placed under the knees or the knees bent so that the feet are flat on the bed.

Explanation to patient: “I need for you to lift your head off of the table. Keep it up as long as possible.”

Notes to examiner: Place your hand under their head (without touching) to provide some cushion if the head drops back. The head should come up and forward, not just up to the ceiling. If the head drops within 10o of neutral, stop the test.

### X. Right & Left Leg Outstretched:

Patient preparation: Patient is supine with a pillow under the head. Both legs must be out straight and shoes off.

Explanation to patient: “I need for you to hold your right leg up. Hold the leg up in this position as long as possible.”

Notes to examiner: Leg must be maintained at 45-50% of hip flexion. If the leg starts to droop, ask the patient to lift the leg up. If the patient lifts the leg up, but cannot maintain that position for 2 seconds, stop the test. Watch for hands under the hips and/or rotation of the leg.

### 2. MG-Composite Scale

For assessment of the MG-Composite scale, the investigator will examine the subject to score all items, except for talking, chewing, and swallowing for which the subject will self-assess. The MG-Composite scale is a validated assessment (Burns et al, 2010 MG Composite and MG-QOL15 Study Group. The MG Composite: A valid and reliable outcome measure for myasthenia gravis. Neurology. 2010 May 4; 74(18):1434-40), with a higher score indicating more severe disease (see FIG. 6) and a 3-point change being of clinical relevance. The scale tests 10 items, with individual items being weighted differently. The overall score ranges from 0 to 50. Clinical personnel must complete mandatory training and be certified to assess subjects' MG-Composite score (details are provided in the Study Procedures Manual).

### 3. Patient-Reported Outcomes

Subjects will complete 4 patient-reported outcomes (PROs) one of which is MGADL, and participate in 1 subject exit interview as per time points mentioned in the schedule of study assessments.

Study personnel other than the treating physician should administer the PROs. The PROs should be completed by the subject themselves in a quiet place.

The PROs and the subject exit interview should be completed in the following order: MG muscle weakness and fatigability, Fatigue, MGADL and MGII, followed by the subject exit interview (which will be performed only at the FV). The PROs should only be checked for completeness. On dosing days, the PROs will be completed prior to dosing.

### MG-Activities of Daily Living (MGADL)

The MGADL is an 8-item PRO instrument developed on the basis of the QMG (Wolfe et al, 1999), see FIG. 7. The MGADL targets symptoms and disability across ocular, bulbar, respiratory, and axial symptoms. In a recent study, reliability, validity, and responsiveness of the MGADL were further assessed. The questionnaire showed strong construct validity when evaluated against the MG-Composite as well as against the MG-QOL15, high test retest reliability in a 1 week interval, and it was demonstrated that a 2-point improvement indicates clinical improvement (Muppidi, 2012; Muppidi et al, 2011). The total MGADL score ranges from 0 to 24, with a higher score indicating more disability. Subjects will complete the MGADL by themselves as described in the standardized administration of PROs.

### Example 3

### Results of MG002 Study

### Study Design

MG0002 (NCT03052751) (protocol provided in Example 2) was a Phase 2 randomized, placebo-controlled, proof-of-concept trial that enrolled 43 MG patients from North America and Europe with generalized muscle weakness and a total Quantitative Myasthenia Gravis score (QMG) of at least 11. MG0002 compared three once/week subcutaneous infusions of placebo (N=22) and 7 mg/kg rozanolixizumab (N=21) on days 1, 8, 15 and were compared during a four-week period (dosing period 1).

After dosing period 1, participants were re-randomized to receive either 7 mg/kg or 4 mg/kg rozanolixizumab on days 29, 36, and 43 (dosing period 2) with continued observation until day 99 (See FIG. 2). Conventional therapies were allowed and included corticosteroids and/or immunomodulatory agents and/or Cholinesterase inhibitors. Pre-specified analyses of safety and efficacy across both dosing periods were looking at the data following six subcutaneous infusions of rozanolixizumab.

### Study Results:

At the end of dosing period 1: The Quantitative Myasthenia Gravis (QMG) responder rate was 38.1% compared to 22.7% for placebo (p=0.223), the Myasthenia Gravis Composite (MGC) responder rate was 47.6% compared to 27.3% for placebo (p=0.144). For the Myasthenia Gravis-Activities of Daily living (MG-ADL), the responder rate was 47.6% compared to 13.6% for placebo (p=0.017). A reduction of 3 or more points from baseline was defined as response for QMG, MGC, and MG-ADL.

The Change from Baseline in the Myasthenia Gravis-Activities of Daily living (MG-ADL) score, an established registration endpoint, was marked improved compared to placebo (p=0.036).

During the dosing period 2, clinically meaningful reductions of the scores were observed with durability of the effects after last dose, until day 99. Generally, the greatest reductions from baseline in QMG, MG-Composite and MG-ADL scores were observed in the rozanolixizumab 7 mg/kg/rozanolixizumab 7 mg/kg group (see FIGS. 3 and 4)

Pre-specified analyses across the two dosing periods (i.e. following six subcutaneous infusions of rozanolixizumab) showed clinically meaningful patient benefit consistently across several disease-specific endpoints, including QMG, MGC and MG-ADL. Participants on active treatment showed a marked reduction of total IgG levels and IgG autoantibody levels. Serum IgG concentrations reduced by 55% after two weeks of rozanolixizumab treatment. Total IgG and anti-acetylcholine receptor (anti-AChR) antibodies decreased by almost 70% from baseline during dosing period 2 in participants receiving rozanolixizumab 7 mg/kg in both dosing periods. Patients with anti-MuSK antibodies were eligible for MG0002 however anti-MuSK antibodies were not assessed due to low patient numbers (n=1 rozanolixizumab 7 mg/kg/rozanolixizumab 7 mg/kg group).

### Example 4: A Phase 3, Randomized, Double-Blind, Placebo-Controlled Study Evaluating Efficacy and Safety of in Adult Patients with Generalized Myasthenia Gravis (MG0003 ClinicalTrials.gov Identifier: NCT03971422)

This is a Phase 3 study of rozanolixizumab in anti-AChR or anti-MuSK autoantibody-positive patients with generalized MG who experience moderate to severe symptoms and are being considered for treatment with IVIg or PLEX. The primary objective of the study is to demonstrate the clinical efficacy of rozanolixizumab in patients with generalized MG. The study is composed of a Screening period of up to 4 weeks, followed by a 6-week doubleblind Treatment Period and an Observation Period of 8 weeks.

Results from MG0002 showed that subcutaneous (sc) administration of rozanolixizumab resulted in a rapid reduction of serum IgG concentrations, which also showed the greatest reductions from Baseline in QMG, MG-Composite, and MG-ADL after 6 doses (Day 50). Therefore, a treatment duration of 6 weeks has been selected for MG0003. An 8-week Observation Period was defined in order to follow the recovery of IgG and AChR and MuSK antibodies, as well as monitoring the sustainability of the clinical effects after discontinuation.

### Number of Participants

The total sample size of the study could range between 150 and 240 study participants.

### Treatment Groups and Duration

The maximum duration of the study per study participants will be up to 18 weeks, consisting of a Screening Period (1 to 28 days to account for central laboratory turn-around time), a Treatment Period (6 weeks), and an Observation Period (8 weeks).

Fixed unit doses across body weight tiers and study arms will be employed.

The placebo arm will be 0.9% sodium chloride aqueous solution (physiological saline, preservative free) for subcutaneous (sc) administration.

Two sc treatment arms will assume fixed unit doses stratified on weight tiers as described below:

Treatment Arm 1 (rozanolixizumab)—6 sc doses at 1 week intervals (equivalent to approximately 7 mg/kg)
Body weight <50 kg: dose to be administered 280 mg
Bodyweight ≥50 kg and <70 kg: dose to be administered 420 mg
Bodyweight ≥70 kg and <100 kg: dose to be administered 560 mg
Bodyweight ≥100 kg; dose to be administered 840 mg

Treatment Arm 2 (rozanolixizumab)—6 sc doses at 1 week intervals (equivalent to approximately 10 mg/kg)
Body weight <50 kg: dose to be administered 420 mg
Bodyweight ≥50 kg and <70 kg: dose to be administered 560 mg
Bodyweight ≥70 kg and <100 kg: dose to be administered 840 mg
Bodyweight ≥100 kg: dose to be administered 1120 mg

A population PKPD model that characterizes the dose-exposure-IgG relationship was used to guide, through simulation, the choice of fixed unit doses at each weight bracket that achieved equivalent IgG reductions (mean and 90% prediction interval) to the weight-based (mg/kg) dosing regimens studied previously. These models-based simulations demonstrate that the proposed weekly doses of rozanolixizumab for 6 consecutive weeks are expected to produce mean maximum IgG reductions of ≥75%.

### Outcome Measures

### Primary Outcome Measures:

1. Change from Baseline to Visit 10 in Myasthenia Gravis-Activities of Daily Living (MG-ADL) score [Time Frame: Baseline and Visit 10 (Day 43)]
   The total MG-ADL score is obtained by summing the responses to each individual item (8 items; Grades: 0, 1, 2, 3). The score ranges from 0 to 24, with a higher score indicating more disability.

### Secondary Outcome Measures:

1. Percentage of participants achieving Myasthenia Gravis-Activities of Daily Living (MG-ADL) response at Visit 10 [Time Frame: Visit 10 (Day 43)]
2. Change from Baseline to Visit 10 in Myasthenia Gravis-Composite score [Time Frame: Baseline and Visit 10 (Day 43)]
   The total Myasthenia Gravis (MG)-composite score is obtained by summing the responses to each individual item (10 items; Grade:0-9 depending on item). The score ranges from 0 to 50, with lower scores indicating lower disease activity.
3. Change from Baseline to Visit 10 in Quantitative Myasthenia Gravis (QMG) score [Time Frame: Baseline and Visit 10 (Day 43)]
   The total QMG score is obtained by summing the responses to each individual item (13 items; Responses: None=0, Mild=1, Moderate=2, Severe=3). The score ranges from 0 to 39, with lower scores indicating lower disease activity.
4. Change from Baseline to Visit 10 in the Myasthenia Gravis (MG) Symptoms Patient Reported Outcome (PRO) ‘Fatigability’ score [Time Frame: Baseline and Visit 10 (Day 43)]
   The MG symptoms PRO instrument consists of 42 items across 5 scales: ocular symptoms (items 1-5); bulbar symptoms (items 6-15); respiratory symptoms (items 16-18); physical fatigue (items 19-33) and muscle weakness fatigability (items 34-42).
   The study participant will be asked to choose the response option that best describes the severity of ocular, bulbar, and respiratory symptoms over the past 7 days using a 4-point Likert scale (“none” to “severe”) and how frequently they experience physical fatigue and muscle weakness fatigability over the past 7 days using a 5-point Likert scale (“none of the time” to “all of the time”), respectively.
5. Change from Baseline to Visit 10 in the Myasthenia Gravis (MG) Symptoms Patient Reported Outcome (PRO) ‘Physical Fatigue, Limb and Axial Weakness’ score [Time Frame: Baseline and Visit 10 (Day 43)]
   The MG symptoms PRO instrument consists of 42 items across 5 scales: ocular symptoms (items 1-5); bulbar symptoms (items 6-15); respiratory symptoms (items 16-18); physical fatigue (items 19-33) and muscle weakness fatigability (items 34-42).
   The study participant will be asked to choose the response option that best describes the severity of ocular, bulbar, and respiratory symptoms over the past 7 days using a 4-point Likert scale (“none” to “severe”) and how frequently they experience physical fatigue and muscle weakness fatigability over the past 7 days using a 5-point Likert scale (“none of the time” to “all of the time”), respectively.
6. Change from Baseline to Visit 10 in the Myasthenia Gravis (MG) Symptoms Patient Reported Outcome (PRO) ‘Bulbar’ score [Time Frame: Baseline and Visit 10 (Day 43)]
   The MG symptoms PRO instrument consists of 42 items across 5 scales: ocular symptoms (items 1-5); bulbar symptoms (items 6-15); respiratory symptoms (items 16-18); physical fatigue (items 19-33) and muscle weakness fatigability (items 34-42).
   The study participant will be asked to choose the response option that best describes the severity of ocular, bulbar, and respiratory symptoms over the past 7 days using a 4-point Likert scale (“none” to “severe”) and how frequently they experience physical fatigue and muscle weakness fatigability over the past 7 days using a 5-point Likert scale (“none of the time” to “all of the time”), respectively.
7. Occurrence of treatment-emergent adverse events (TEAEs) [Time Frame: From Baseline until End of Study Visit (up to Week 14)]
   An Adverse Event (AE) is any untoward medical occurrence in a patient or clinical investigation subject administered a pharmaceutical product, which does not necessarily have a causal relationship with this treatment. An AE could therefore be any unfavorable and unintended sign, symptom, or disease temporally associated with the use of a medicinal (investigational) product, whether or not related to the medicinal (investigational) product.
   A Treatment emergent adverse event (TEAE) is defined as any event that was not present prior to the first administration of IMP or any unresolved event already present before the first administration of IMP that worsens in intensity following exposure to treatment.
8. Treatment-emergent adverse events (TEAEs) leading to withdrawal of investigational medicinal product (IMP) [Time Frame: From Baseline until End of Study Visit (up to Week 14)]
   One of the secondary outcome measures is to assess safety and tolerability of the IMP in the MG patients. This can be measured by Treatment emergent adverse events (TEAEs) leading to withdrawal of IMP.
   A TEAE is defined as any event that was not present prior to the first administration of IMP or any unresolved event already present before the first administration of IMP that worsens in intensity following exposure to treatment.

### Eligibility Criteria

### Criteria

### Inclusion Criteria:

Study participant must be ≥18 years of age, at the time of signing the informed consent

Study participant has documented diagnosis of generalized myasthenia gravis (gMG) at Visit 1, based on study participant's history and supported by previous evaluations

Study participant has a confirmed positive record of autoantibodies against acetylcholine receptor (AChR) or muscle-specific kinase (MuSK) prior to Visit 1

Study participant has Myasthenia Gravis Foundation of America (MGFA) Class II to IVa at Visit 1

Study participant with a myasthenia gravis-activities of daily living (MG-ADL) score of at least 3 AND a quantitative myasthenia gravis (QMG) score of at least 11 at Visit 1 and at Baseline

### Exclusion Criteria:

Study participant has a clinically relevant active infection (eg, sepsis, pneumonia, or abscess) in the opinion of the Investigator, or had a serious infection (resulting in hospitalization or requiring parenteral antibiotic treatment) within 6 weeks prior to the first dose of investigational medicinal product (IMP)

Study participant has experienced hypersensitivity reaction after exposure to other anti-neonatal Fc receptor (FcRn) drugs

Study participant with severe (defined as Grade 3 on the MG-ADL scale) weakness affecting oropharyngeal or respiratory muscles, or who has myasthenic crisis or impending crisis a Visit 1

### Permitted Concomitant Treatments

### Permitted

### Medications

### Dose Comment

### Oral Corticosteroids

(eg, prednisolone)
Stable for 2 weeks prior to

### Baseline

Methotrexate ≤30 mg/week Treated for previous 6 months and
on stable dose for 2 months prior

### to Baseline

Mycophenolate mofetil ≤3 g/day Treated for previous 6 months and
on stable dose for 2 months prior

### to Baseline

Cyclosporina ≤5 mg/kg/day for
unmodified
≤4 mg/kg/day for modified
(microemulsion)
Treated for previous 6 months and
on stable dose for 2 months prior

### to Baseline

Azathioprine ≤3 mg/kg/day Treated for previous 6 months and
on stable dose for 2 months prior

### to Baseline

### Cholinesterase

inhibitors
≤600 mg

### Pyridostigmine/day

Stable dose not required—dose
held on days of efficacy outcomes
Tacrolimusb ≤5 mg/day Treated for previous 6 months and
on stable dose for 2 months prior

### to Baseline

a Doses higher than listed are permissible if trough level is ≤300 ng/L.
b If the total daily weight-based dose is >5 mg, then a plasma trough level should be checked to ensure study participant is not above the recommended therapeutic range.

### Prohibited Concomitant Treatments (Medications and Therapies)

The following concomitant medications are prohibited during the study:

All biologics including rituximab

### Cyclophosphamide

### Pimecrolimus

### IPP-201101 (Lupuzor™)

### Immunoadsorption

### REFERENCES

Barnett C, Katzberg H, Nabavi M, Bril V. The quantitative myasthenia gravis score: comparison with clinical, electrophysiological, and laboratory markers. J Clin Neuromuscul Dis. 2012; 13(4):201-5.
Burns T M, Conaway M, Sanders D B; MG Composite and MG-QOL15 Study Group. The MG Composite: A valid and reliable outcome measure for myasthenia gravis. Neurology. 2010 May 4; 74(18):1434-40.
CPMP/ICH/135/95 Note for guidance on Good Clinical Practice (EMEA) July 2002.
Gilhus N E, Verschuuren J J. Myasthenia gravis: subgroup classification and therapeutic strategies. Lancet Neurol. 2015; 14(10):1023-36.
Jaretzki A, Barohn R J, Ernstoff M D, Kaminski H J, Keesey M D, Penn A S, et al. Myasthenia gravis: Recommendations for clinical research standards. Neurol. 2000; 55:16-23.
Muppidi S. The myasthenia gravis-specific activities of daily living profile. Ann N Y Acad Sci, 2012. 1274: p. 114-9
Muppidi S, Wolfe G I, Conaway M, Burns T M; MG COMPOSITE AND MG-QOL15 STUDY GROUP. MG-ADL: still a relevant outcome measure. Muscle Nerve. 2011 November; 44(5):727-31.
Wolfe G I, Herbelin L, Nations S P, Foster B, Bryan W W, Barohn R J. Myasthenia gravis activities of daily living profile. Neurology. 1999 Apr. 22; 52(7):1487-9.
World Health Organization. Guidelines for treatment of tuberculosis (Fourth edition). Geneva: World Health Organization; 2010.

All references cited herein are hereby incorporated by reference in their entireties. The foregoing examples are meant to illustrate the invention, not limit it in any way. Modifications within the spirit and scope of the invention disclosed herein are included.

1. A method of treating or preventing myasthenia gravis (MG) in a human in need thereof, the method comprising administering to the human at least three doses of an anti-FcRn antibody or antigen binding fragment thereof comprising
a heavy chain or heavy chain fragment having a variable region, wherein said variable region comprises three CDRs having the sequences given in SEQ ID NO: 1 for CDR H1, SEQ ID NO: 2 for CDR H2 and SEQ ID NO: 3 for CDR H3, and
a light chain or light chain fragment having a variable region, wherein said variable region comprises three CDRs having the sequences given in SEQ ID NO: 4 for CDR L1, SEQ ID NO: 5 for CDR L2 and SEQ ID NO: 6 for CDR L3,
wherein each dose is independently selected from 4 mg/kg, 7 mg/kg, 10 mg/kg, 15 mg/kg and 20 mg/kg.

2. (canceled)

3. (canceled)

4. (canceled)

5. The method according to claim 1, wherein the antibody or antigen binding fragment thereof is humanized.

6. The method according to claim 1 wherein the anti-FcRn antibody or binding fragment thereof comprises a heavy chain comprising the sequence given in SEQ ID NO:29 or a sequence specific to FcRn at least 80% identical thereto.

7. The method according to claim 1 wherein the anti-FcRn antibody or binding fragment thereof comprises a light chain comprising the sequence given in SEQ ID NO:15 or a sequence specific to FcRn at least 80% identical thereto.

8. The method according to claim 1 wherein the anti-FcRn antibody or binding fragment thereof comprises a heavy chain variable domain sequence having the sequence given in SEQ ID NO:29 and a light chain variable domain sequence comprising the sequence given in SEQ ID NO:15.

9. The method according to claim 1, wherein the antibody is a full length antibody.

10. The method according to claim 9 wherein the full length antibody is selected from the group consisting of an IgG1, IgG4 and IgG4P.

11. The method according to claim 9 wherein the anti-FcRn antibody has a heavy chain comprising the sequence given in SEQ ID NO:72 or SEQ ID NO:87 or SEQ ID NO:43 and a light chain comprising the sequence given in SEQ ID NO:22.

12. The method according to claim 1 wherein the anti-FcRn antibody is UCB7665 (rozanolixizumab).

13. The method according to claim 1 having a binding affinity for human FcRn of 100 pM or less.

14. The method according to claim 13 wherein the binding affinity for human FcRn is 100 pM or less when measured at pH6 and at pH7.4.

15. The method according to claim 1 wherein the antibody or antigen binding fragment is provided as a pharmaceutical composition comprising one or more of a pharmaceutically acceptable excipient, diluent or carrier.

16. The method according to claim 15, wherein the pharmaceutical composition further comprises one or more other active ingredients.

17. (canceled)

18. The method according to claim 1, wherein each dose is 7 mg/kg or 10 mg/kg.

19. The method according to claim 1, wherein each dose is administered weekly.

20. The method according to claim 1, wherein at least 4, 5 or 6 doses are administered weekly.

21. The method according to claim 1, wherein at least 6 doses of antibody or antigen binding fragment are administered weekly and each dose is in the range of 4 mg/kg to 30 mg/kg.

22. The method according to claim 1 wherein the anti-FcRn antibody or antigen binding fragment thereof is administered subcutaneously or intravenously.

23. (canceled)

24. (canceled)

25. The method according to claim 1 wherein four, five or six weekly doses are administered, followed by one or more additional doses that are lower and/or less frequent than the initial four, five or six doses.

26. (canceled)

27. The method according to claim 1 wherein the human is suffering from generalised MG which is classified as moderate to severe.

28. The method according to claim 1 wherein the human is anti-AChR and/or anti-MuSK autoantibody-positive.

29. The method according to claim 1 wherein each dose is provided as a fixed unit dose selected from 280 mg, 420 mg, 560 mg, 840 mg and 1120 mg.

30. A method of treating or preventing myasthenia gravis (MG) in a human in need thereof, the method comprising administering to the human at least 3 doses of an anti-FcRn antibody or antigen-binding fragment thereof wherein each dose is selected from 280 mg, 315 mg, 350 mg, 385 mg, 420 mg, 455 mg, 490 mg, 525 mg, 560 mg, 595 mg, 630 mg, 665 mg, 700 mg, 735 mg, 770 mg, 805 mg, 840 mg, 875 mg, 910 mg, 945 mg, 980 mg, 1015 mg, 1050 mg, 1085 mg and 1120 mg and wherein the anti-FcRn antibody or antigen binding fragment thereof comprises a heavy chain comprising the sequence given in SEQ ID NO:29 and a light chain comprising the sequence given in SEQ ID NO:15.

31. A method of treating or preventing myasthenia gravis (MG) in a human in need thereof, the method comprising administering to the human at least 6 doses of an anti-FcRn antibody or antigen-binding fragment thereof wherein for a body weight of less than 50 kg the dose is 280 mg, for a body weight of equal to or greater than 50 kg but less than 70 kg the dose is 420 mg, for a body weight of equal to or greater than 70 kg but less than 100 kg the dose is 560 mg and for a body weight of equal to or greater than 100 kg the dose is 840 mg.

32. A method of treating or preventing myasthenia gravis (MG) in a human in need thereof, the method comprising administering to the human at least 6 doses of an anti-FcRn antibody or antigen-binding fragment thereof wherein for a body weight of less than 50 kg the dose is 420 mg, for a body weight of equal to or greater than 50 kg but less than 70 kg the dose is 560 mg, for a body weight of equal to or greater than 70 kg but less than 100 kg the dose is 840 mg and for a body weight of equal to or greater than 100 kg the dose is 1120 mg.

33. The method according to claim 30 wherein each dose is administered weekly.
